(19) United States
(12) Patent Application Publication
(10) Pub. No.: US 2026/0284268 A1
(43) Pub. Date: Sep. 24, 2026

(54) Self-Polymerizing, Radiopaque Resin For Percutaneous Vertebroplasty

(71) Applicants: Matthew L. Becker, Chapel Hill, NC (US); Russel E. Thompson, Durham, NC (US); Maddison Segal, Durham, NC (US)

(72) Inventors: Matthew L. Becker, Chapel Hill, NC (US); Russel E. Thompson, Durham, NC (US); Maddison Segal, Durham, NC (US)

(73) Assignee: Duke University, Durham, NC (US)

(21) Appl. No.: 19/574,129

(22) Filed: Mar. 20, 2026

Related U.S. Application Data
(60) Provisional application No. 63/775,953, filed on Mar. 21, 2025.

(51) Int. Cl. A61L 27/50; A61L 27/38; A61L 27/44

(52) U.S. Cl. CPC A61L 27/50; A61L 27/3834; A61L 27/446; A61L 2430/02

(57) ABSTRACT

In various embodiments, the present invention relates to a solvent-free polymer system using poly(allyl glycidyl ether succinate) (PAGES) for vertebral augmentation. PAGES crosslinks in situ through thiol-ene click chemistry with a cure time at 37° C. ranging from 17 to 53 minutes based on degree of polymerization and crosslinker concentration. The addition of SrCO₃ increased the ultimate compressive strength (σₘₐₓ) of the PAGES composite to 4.4±0.4 MPa. Furthermore, the addition of SrCO₃ increases osteoblast proliferation and differentiation of mesenchymal stem cells seeded onto the surface of PAGES composite. Finally, the compressive strength of fractured rabbit vertebra was increased ex vivo when filled with the PAGES composite demonstrating its potential as a bone augmentation material.

### CROSS REFERENCE TO RELATED APPLICATIONS

This application claims the benefit of U.S. provisional patent application Ser. No. 63/775,953 entitled “Self-Polymerizing, Radiopaque Resin For Percutaneous Vertebroplasty” filed Mar. 21, 2025, and incorporated herein by reference in its entirety.

### STATEMENT REGARDING FEDERALLY SPONSORED RESEARCH OR DEVELOPMENT SUPPORT

This invention was made with government support under HL159954 awarded by the National Institutes of Health. The government has certain rights in the invention.

### FIELD OF THE INVENTION

One or more embodiments of the present invention relates to a solvent free, injectable, radiopaque composite network. In certain embodiments, the present invention is directed to a percutaneous poly(allyl glycidyl ether succinate) (PAGES)-based polymeric cement for vertebral augmentation.

### BACKGROUND OF THE INVENTION

Vertebral compression fractures (VCFs) arise from normal loading on an abnormal vertebra or traumatic loading on a normal vertebra. VCFs can be a source of chronic back pain and morbidity, particularly in older adults and is estimated to cost the United States healthcare system more than $1 Billion per year. VCFs most commonly result from either osteoporosis, a disease that weakens bones, or bone destruction caused by cancerous or benign masses. Treatment for VCFs can vary from medication and physical therapy to invasive intervention with open surgical fusion or percutaneous vertebral augmentation. There are two different percutaneous augmentation strategies in widespread use: vertebroplasty and kyphoplasty. (See, Y. Robinson, C. E. Heyde, P. Forsth, C. Olerud, “Kyphoplasty in osteoporotic vertebral compression fractures—Guidelines and technical considerations,” Journal of Orthopaedic Surgery and Research 6 (2011) 43. https://doi.org/10.1186/1749-799X-6-43, the disclosure of which is incorporated herein by reference in its entirety).

Augmentation is accomplished through one or two small incisions in the back through which a self-tapping drill and cannula are introduced. Conventionally, both surgical procedures use poly(methyl methacrylate) (PMMA) or calcium phosphate cement to fill the void within the VCF, however kyphoplasty utilizes a balloon to restore the height and shape of the vertebra prior to injection. Once this is done, the surgeon then injects a cement mixture into the cavity to fill it in and further stabilize the area. The procedure is performed under local anesthesia to minimize any discomfort for the patient. X-rays or CT scans are also used as a guide to inject the cement mixture into the affected area accurately.

Recently, kyphoplasty has also been found to confer a survival benefit compared to conservative management. (See, e.g., J. A. Hirsch, R. V. Chandra, N. S. Carter, D. Beall, M. Frohbergh, K. Ong, “Number Needed to Treat with Vertebral Augmentation to Save a Life,” AJNR. American Journal of Neuroradiology 41 (2020) 178-182. https://doi.org/10.3174/AJNR. A6367, the disclosure of which is incorporated herein by reference in its entirety). Percutaneous augmentation procedures have a significantly lower rate of surgical complications compared to open spinal fusion procedures. (See, e.g., A.-R. Cho, S.-B. Cho, J.-H. Lee, K.-H. Kim, “Experimental Study Effect of Augmentation Material Stiffness on Adjacent Vertebrae after Osteoporotic Vertebroplasty Using Finite Element Analysis with Different Loading Methods,” Pain Physician 18 (2015) E1101-10, the disclosure of which is incorporated herein by reference in its entirety). Additionally, for patients with fractures due to cancerous masses, the tumor can be ablated during the vertebral augmentation procedure, adding some localized cancer control. (See, e.g., A. Ragheb, A. Vanood, D. K. Fahim, “The Addition of Radiofrequency Tumor Ablation to Kyphoplasty May Reduce the Rate of Local Recurrence in Spinal Metastases Secondary to Breast Cancer,” World Neurosurgery 161 (2022) e500-e507. https://doi.org/10.1016/J. WNEU.2022.02.052, the disclosure of which is incorporated herein by reference in its entirety). Due to these clinical advantages, vertebral augmentation is an increasingly common procedure to address VCFs.

In current practice, a powdered solid of barium sulfate (BaSO₄), for radiopacity, and poly(methyl methacrylate) (PMMA) is mixed with a liquid portion containing methyl methacrylate (MMA) monomers, stabilizers and a radical initiator. This polymeric cement slurry is then injected into the vertebral void space where it cures over time until fully solidified. Despite its widespread use as an augmentation material, PMMA has multiple drawbacks. Among the reported complications, there are subsequent fractures in both previously operated and in the adjacent vertebrae, likely due to the addition of the material that is stiffer than the surrounding trabecular bone. PMMA has a compressive modulus and peak compressive strength that are approximately 100-fold greater than native vertebral bodies. (See, e.g., S. Saha, S. Pal, “Mechanical properties of bone cement: A review,” Journal of Biomedical Materials Research 18 (1984) 435-462. https://doi.org/10.1002/jbm.820180411; X. Banse, T. J. Sims, A. J. Bailey, “Mechanical Properties of Adult Vertebral Cancellous Bone: Correlation With Collagen Intermolecular Cross-Links,” Journal of Bone and Mineral Research 17 (2002) 1621-1628. https://doi.org/10.1359/jbmr.2002.17.9.1621, the disclosures of which are incorporated herein by reference in their entirety). Fahim et al. (see, e.g., A. Ragheb, A. Vanood, D. K. Fahim, “The Addition of Radiofrequency Tumor Ablation to Kyphoplasty May Reduce the Rate of Local Recurrence in Spinal Metastases Secondary to Breast Cancer,” World Neurosurgery 161 (2022) e500-e507. https://doi.org/10.1016/J. WNEU.2022.02.052, the disclosure of which is incorporated herein by reference in its entirety) have suggested that aggressive cement augmentation of the vertebral body may promote the endplate deflection fracture mechanism. Biomechanical and finite element studies have demonstrated that vertebroplasty alters load transfer, resulting in increased stresses in adjacent endplates as well.

These mechanical property discrepancies are thought to contribute to the development of fractures in adjacent vertebral bodies following augmentation, occurring in 7-15% of patients. (See, e.g., A. Kircelli, I. Çöven, “Percutaneous Balloon Kyphoplasty Vertebral Augmentation for Compression Fracture Due to Vertebral Metastasis: A 12-Month Retrospective Clinical Study in 72 Patients,” Medical Science Monitor: International Medical Journal of Experimental and Clinical Research 24 (2018) 2142-2148. https://doi.org/10.12659/MSM.909169; Y. T. Wang, X. T. Wu, H. Chen, C. Wang, Z. Bin Mao, “Adjacent-level symptomatic fracture after percutaneous vertebral augmentation of osteoporotic vertebral compression fracture: a retrospective analysis,” Journal of Orthopaedic Science 19 (2014) 868-876. https://doi.org/i0.1007/S007_76-014-0610-7; A. Boger, P. Heini, M. Windolf, E. Schneider, “Adjacent vertebral failure after vertebroplasty: a biomechanical study of low-modulus PMMA cement,” Eur Spine J 16 (2007) 2118-2125. https://doi.org/10.1007/s00586-007-0473-0, the disclosures of which are incorporated herein by reference in their entirety).

Additionally, PMMA is biologically inert and nondegradable, the polymerization reaction within the vertebral body is highly exothermic and MMA monomers have been shown to induce neurotoxicity. (See, e.g., T. D. Williams, T. Adler, L. Smokoff, A. Kaur, B. Rodriguez, K. J. Prakash, E. Redzematovic, T. S. Baker, B. I. Rapoport, E. S. Yoon, D. P. Beall, J. S. Dordick, R. A. De Leacy, “Bone Cements Used in Vertebral Augmentation: A State-of-the-art Narrative Review,” Journal of Pain Research 17 (2024) 1029-1040. https://doi.org/10.2147/JPR.S437827; U. J. Schlegel, M. Sturm, P. Eysel, S. J. Breusch, “Pre-Packed Vacuum Bone Cement Mixing Systems. A Further Step in Reducing Methylmethacrylate Exposure in Surgery,” The Annals of Occupational Hygiene 54 (2010) 955-961. https://doi.org/10.1093/annhyg/meq045, the disclosures of which are incorporated herein by reference in their entirety). These factors are thought to contribute to an absence of new bone growth around and within the PMMA block.

Ceramics and metal salts are often added to augmentation materials to alter biological effects and afford radiopacity. Radiopacity is essential so that the material can be easily identified using fluoroscopic guidance during the augmentation procedure. Barium sulfate (BaSO₄) is the current standard additive used to make PMMA radiopaque and is known to be tolerated by patients. Other ceramic additives have been shown to promote osteogenesis, including strontium carbonate (SrCO₃) and alpha tricalcium phosphate (α-TCP). (See, e.g., T. Ahlfeld, A. Lode, A. M. Placht, T. Fecht, T. Wolfram, S. Grom, A. Hoess, C. Vater, C. Brauer, S. Heinemann, G. Lauer, F. Reinauer, M. Gelinsky, “A comparative analysis of 3D printed scaffolds consisting of poly(lactic-co-glycolic) acid and different bioactive mineral fillers: aspects of degradation and cytocompatibility,” Biomaterials Science 11 (2023) 5590-5604. https://doi.org/10.1039/D2BM02071H; H. H. Xu, P. Wang, L. Wang, C. Bao, Q. Chen, M. D. Weir, L. C. Chow, L. Zhao, X. Zhou, M. A. Reynolds, “Calcium phosphate cements for bone engineering and their biological properties,” Bone Res 5 (2017) 1-19. https://doi.org/10.1038/boneres.2017.56, the disclosures of which are incorporated herein by reference in their entirety).

Additionally, SrCO₃ doped cements have been found to promote osteoblast attachment and osseointegration. (See, e.g., F. Zhao, W. W. Lu, K. D. K. Luk, K. M. C. Cheung, C. T. Wong, J. C. Y. Leong, K. D. Yao, “Surface treatment of injectable strontium-containing bioactive bone cement for vertebroplasty,” Journal of Biomedical Materials Research. Part B, Applied Biomaterials 69 (2004) 79-86. https://doi.org/10.1002/JBM.B.20041; H. Autefage, F. Allen, H. M. Tang, C. Kallepitis, E. Gentleman, N. Reznikov, K. Nitiputri, A. Nommeots-Nomm, M. D. O'Donnell, C. Lange, B. M. Seidt, T. B. Kim, A. K. Solanki, F. Tallia, G. Young, P. D. Lee, B. F. Pierce, W. Wagermaier, P. Fratzl, A. Goodship, J. R. Jones, G. Blunn, M. M. Stevens, “Multiscale analyses reveal native-like lamellar bone repair and near perfect bone-contact with porous strontium-loaded bioactive glass,” Biomaterials 209 (2019) 152-162. https://doi.org/10.1016/j.biomaterials.2019.03.035, the disclosures of which are incorporated herein by reference in their entirety). Finally, SrCO₃ containing cements have been used in vertebroplasty in patients with similar levels of pain relief (See, e.g., K. M. C. Cheung, W. W. Lu, K. D. K. Luk, C. T. Wong, D. Chan, J. X. Shen, G. X. Qiu, Z. M. Zheng, C. H. Li, S. L. Liu, W. K. Chan, J. C. Y. Leong, “Vertebroplasty by use of a strontium-containing bioactive bone cement,” Spine 30 (2005). https://doi.org/10.1097/01. BRS.0000175183.57733. E5, the disclosure of which is incorporated herein by reference in its entirety).

Other materials currently under investigation for use in bone augmentation systems include calcium phosphate cements (CPCs) which show promise for improved biocompatibility. (See, e.g., H. H. Xu, P. Wang, L. Wang, C. Bao, Q. Chen, M. D. Weir, L. C. Chow, L. Zhao, X. Zhou, M. A. Reynolds, “Calcium phosphate cements for bone engineering and their biological properties,” Bone Res 5 (2017) 1-19. https://doi.org/10.1038/boneres.2017.56, the disclosure of which is incorporated herein by reference in its entirety). Unfortunately, many CPCs have long setting times and can experience phase separation which has further hampered their clinical translation. (See, e.g., T. D. Williams et al., “Bone Cements Used in Vertebral Augmentation: A State-of-the-art Narrative Review,” Journal of Pain Research 17 (2024) 1029-1040. https://doi.org/10.2147/JPR.S437827; C. Li, L. Gao, F. Chen, C. Liu, “Fabrication of mesoporous calcium silicate/calcium phosphate cement scaffolds with high mechanical strength by freeform fabrication system with micro-droplet jetting,” J Mater Sci 50 (2015) 7182-7191. https://doi.org/10.1007/s10853-015-9244-1, the disclosures of which are incorporated herein by reference in their entirety).

Methods have also been explored to soften native PMMA bone cement through the incorporation of non-miscible phases during polymerization, such as linoleic acid or hyaluronic acid. (See, e.g., A. Boger, M. Bohner, P. Heini, S. Verrier, E. Schneider, Properties of an injectable low modulus PMMA bone cement for osteoporotic bone, Journal of Biomedical Materials Research Part B: Applied Biomaterials 86B (2008) 474-482. https://doi.org/10.1002/jbm.b.31044; M. Hossain, J. H. Jeong, T. Sultana, J. H. Kim, J. E. Moon, S. Im, A composite of polymethylmethacrylate, hydroxyapatite, and β-tricalcium phosphate for bone regeneration in an osteoporotic rat model, Journal of Biomedical Materials Research Part B: Applied Biomaterials 111 (2023) 1813-1823. https://doi.org/10.1002/jbm.b.35287; C. Robo, D. Wenner, S. J. K. A. Ubhayasekera, J. Hilborn, C. Öhman-Magi, C. Persson, Functional Properties of Low-Modulus PMMA Bone Cements Containing Linoleic Acid, Journal of Functional Biomaterials 12 (2021) 5. https://doi.org/10.3390/jfb12010005, the disclosures of which are incorporated herein by reference in their entirety).

More recently, Yang et al. incorporated microcapsules of paraffin into the cement to mitigate the effect of the exothermic polymerization on the surrounding vertebrae. After 24 h of injection, PMMA cement with 20% microcapsules showed a significantly smaller thermal zone of necrosis. (See, e.g., S. Ramanathan, Y.-C. Lin, S. Thirumurugan, C.-C. Hu, Y.-F. Duann, R.-J. Chung, Poly(methyl methacrylate) in Orthopedics: Strategies, Challenges, and Prospects in Bone Tissue Engineering, Polymers 16 (2024) 367. https://doi.org/10.3390/polym16030367, the disclosure of which is incorporated herein by reference in its entirety). Within PMMA cements groups have looked to delivery active compounds and integrin-specific peptides in order to aid in bone regeneration. (See, e.g., A. Y. Clark, K. E. Martin, J. R. Garcia, C. T. Johnson, H. S. Theriault, W. M. Han, D. W. Zhou, E. A. Botchwey, A. J. Garcia, “Integrin-specific hydrogels modulate transplanted human bone marrow-derived mesenchymal stem cell survival, engraftment, and reparative activities,” Nat Commun 11 (2020) 114. https://doi.org/10.1038/s41467-019-14000-9; F. Boschetto, T. Honma, T. Adachi, N. Kanamura, W. Zhu, T. Yamamoto, E. Marin, G. Pezzotti, “Development and evaluation of osteogenic PMMA bone cement composite incorporating curcumin for bone repairing,” Materials Today Chemistry 27 (2023) 101307. https://doi.org/10.1016/j.mtchem.2022.101307, the disclosures of which are incorporated herein by reference in their entirety). Many reports to date look to augment the standard PMMA resin through additives or introduction of pores but little work looks for polymeric network alternatives.

What is needed in the art is a bone augmentation material having mechanical properties similar to native bone that will solidify on a timeframe that is compatible with current interventional procedures, that does not result in thermal necrosis of patient tissue, that affords earlier patient mobility, and that, ideally, promotes the deposition of new bone.

### SUMMARY OF THE INVENTION

In various embodiments, the present invention relates to a bone augmentation material having mechanical properties similar to native bone that will solidify on a timeframe that is compatible with current interventional procedures, afford earlier patient mobility, ins not exothermic, and promotes the deposition of new bone. In various embodiments, the present invention is directed to poly(allyl glycidyl ether succinate) (PAGES)-based composite networks and their use as a percutaneous polymeric cement for vertebral augmentation. Advantageously, these PAGES-based copolymer composite networks have been found to have mechanical properties near that of native vertebra and should not, therefore, contribute to longer term complications due to the mechanical mismatch common to PMMA-based systems. These composite networks contain PAGES copolymers that are crosslinked with a multi-functional thiol crosslinker, such as trimethylolpropane tris(3-mercaptopropionate), via a thiol-ene addition mechanism, and contain one or more ceramic compounds that make the networks radiopaque. The rate of crosslinking can be controlled via the concentration of the crosslinker and the temperature. In one or more embodiments, the composite networks may contain from about 30% to about 80% radiopaque materials by mass, without substantially affecting its crosslinking mechanism or set time. These ceramic compounds also assist with the proliferation and differentiation of mesenchymal stem cells seeded onto the surface of the PAGES-based composite.

Moreover, the PAGES copolymers possess a low neat viscosity that scales with the degree of polymerization, affording injectability without the addition of solvents or diluents. (See, Segal M I, Bahnick A J, Judge N G, Becker M L. “Synthesis and Solvent Free D LP 3D Printing of Degradable Poly(Allyl Glycidyl Ether Succinate).” Angew Chem Int Ed Engl. Published online Oct. 25, 2024. doi:10.1002/ANIE.202414016, the disclosure of which is incorporated herein by reference) and are not endothermic, thereby avoiding the risk of thermal damage to surrounding tissues. The absence of solvent likewise avoids a major limitation of many possible polymer formulations.

Additionally, the thiol-ene “click” crosslinking reaction is known to be cell compatible, does not generate residual by-products, and has been applied in many other biomaterial applications. (See, e.g., Kharkar P M, Rehmann M S, Skeens K M, Maverakis E, Kloxin A M. Thiol-ene click hydrogels for therapeutic delivery. ACS Biomater Sci Eng. 2016; 2(2):165-179. doi:10.1021/ACSBIOMATERIALS.5B00420; Chang C Y, Johnson H C, Babb O, Fishel M L, Lin CC. Biomimetic stiffening of cell-laden hydrogels via sequential thiol-ene and hydrazone click reactions. Acta Biomater. 2021; 130:161. doi:10.1016/J. ACTBIO.2021.05.054; Machado T O, Sayer C, Araujo P H H. Thiol-ene polymerisation: A promising technique to obtain novel biomaterials. Eur Polym J. 2017; 86:200-215. doi:10.1016/J. EURPOLYMJ.2016.02.025; and Ganabady K, Contessi Negrini N, Scherba J C, et al. High-Throughput Screening of Thiol-ene Click Chemistries for Bone Adhesive Polymers. ACS Appl Mater Interfaces. 2023; 15(44):50908-50915. doi:10.1021/ACSAMI.3C12072/SUPPL_FILE/AM3C12072_SI_002. MP4, the disclosures of which are incorporated herein by reference in their entireties.

In a first aspect, the present invention is directed to a solvent free, injectable, radiopaque composite network comprising: a poly(allyl glycidyl ether succinate) copolymer comprising one or more allyl functional groups and a residue of a mono, di, or multi-functional initiating alcohol; a multi-functional thiol crosslinking compound; and a plurality of radiopaque ceramics or metal salt molecules. In some of these embodiments, the poly(allyl glycidyl ether succinate) copolymer is the reaction product of succinic anhydride, allyl glycidyl ether, and a mono or di-functional initiating alcohol, and is formed by ring-opening polymerization using Mg(BHT)₂(THF)₂ as a catalyst.

In some embodiments, the solvent free, injectable, radiopaque composite network of the present invention includes any one or more of the above referenced embodiments of the first aspect of the present invention wherein the poly(allyl glycidyl ether succinate) copolymer has a single poly(allyl glycidyl ether succinate) copolymer chain covalently bonded to the residue of a mono-functional initiating alcohol. In some other embodiments, the solvent free, injectable, radiopaque composite network of the present invention includes any one or more of the above referenced embodiments of the first aspect of the present invention wherein the poly(allyl glycidyl ether succinate) copolymer has two poly(allyl glycidyl ether succinate) copolymer chain covalently bonded to the residue of a di-functional initiating alcohol. In various embodiments, the solvent free, injectable, radiopaque composite network of the present invention includes any one or more of the above referenced embodiments of the first aspect of the present invention having a degree of polymerization of from about 10 to about 60, preferably from about 25 to about 55, and more preferably from about 29 to about 49.

In certain embodiments, the solvent free, injectable, radiopaque composite network of the present invention includes any one or more of the above referenced embodiments of the first aspect of the present invention wherein the mono-functional initiating alcohol is selected from the group consisting of propargyl alcohol, benzyl alcohol, allyl alcohol, 4-dibenzyocyclooctynol, 4-hydroxybutan-2-one, 3-hydroxypropan-2-one, 5-hydroxypentan-2-one, 6-hydroxyhexan-2-one, 7-hydroxyheptan-2-one, 8-hydroxyoctan-2-one, 5-norbornen-2-ol, α-bromoisobtyryl 4-methanol benzylmethanoate, and combinations thereof. In some embodiments, the solvent free, injectable, radiopaque composite network of the present invention includes any one or more of the above referenced embodiments of the first aspect of the present invention wherein the di-functional initiating alcohol is selected from the group consisting of 1,2-ethanediol, 1,3-propane diol, 1,4-butane diol, 1_,5-pentane diol, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol, 1,19-nonadecanediol, 1,20-icosanediol, and combinations thereof.

In one or more embodiments, the solvent free, injectable, radiopaque composite network of the present invention includes any one or more of the above referenced embodiments of the first aspect of the present invention having the formula:

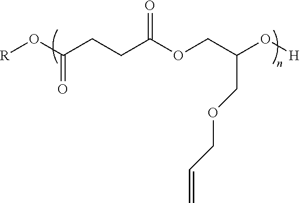

where n is an integer from about 10 to about 60 and R is a residue of an initiating alcohol. In some other embodiments, the solvent free, injectable, radiopaque composite network of the present invention includes any one or more of the above referenced embodiments of the first aspect of the present invention having the formula:

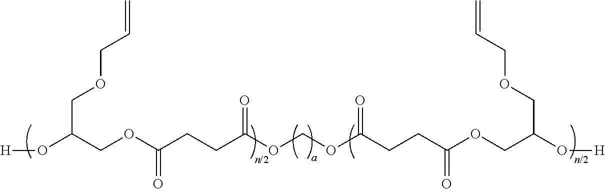

where n is an integer from about 10 to about 60 and where a is an integer from about 2 to about 12.

In some embodiments, the solvent free, injectable, radiopaque composite network of the present invention includes any one or more of the above referenced embodiments of the first aspect of the present invention wherein the multi-functional thiol crosslinking compound selected from the group consisting of trimethylolpropane tris(3-mercaptopropionate), trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol), and combinations thereof. In certain embodiments, the solvent free, injectable, radiopaque composite network of the present invention includes any one or more of the above referenced embodiments of the first aspect of the present invention the multi-functional thiol crosslinking compound is trimethylolpropane tris(3-mercaptopropionate).

In one or more embodiments, the solvent free, injectable, radiopaque composite network of the present invention includes any one or more of the above referenced embodiments of the first aspect of the present invention wherein the multi-functional thiol crosslinking compound has the formula:

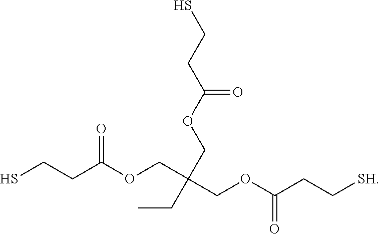 [SMILES: CCC(COC(=O)CCS)(COC(=O)CCS)COC(=O)CCS]

In various embodiments, the solvent free, injectable, radiopaque composite network of the present invention includes any one or more of the above referenced embodiments of the first aspect of the present invention having a consumption percentage of from about 30% to about 60%, preferably from about 35% to about 60%, and more preferably from about 40% to about 55% of the solvent free, injectable, radiopaque composite network.

In one or more embodiments, the solvent free, injectable, radiopaque composite network of the present invention includes any one or more of the above referenced embodiments of the first aspect of the present invention wherein the plurality of radiopaque ceramics or metal salt molecules comprise a radiopaque ceramic or metal salt of strontium, barium, calcium, or a combination thereof. In various embodiments, the solvent free, injectable, radiopaque composite network of the present invention includes any one or more of the above referenced embodiments of the first aspect of the present invention wherein the plurality of radiopaque ceramic molecules are selected from the group consisting of strontium carbonate, barium sulfate, α-alpha tricalcium phosphate (α-TCP), and combinations thereof. In certain embodiments, the solvent free, injectable, radiopaque composite network of the present invention includes any one or more of the above referenced embodiments of the first aspect of the present invention wherein the plurality of radiopaque ceramic molecules comprise strontium carbonate.

In one or more embodiments, the solvent free, injectable, radiopaque composite network of the present invention includes any one or more of the above referenced embodiments of the first aspect of the present invention wherein the plurality of radiopaque ceramic molecules comprise from about 10% to about 90%, preferably from about 30% to about 80%, and more preferably from about 30% to about 60% by mass of the solvent free, injectable, radiopaque composite network. In various embodiments, the solvent free, injectable, radiopaque composite network of the present invention includes any one or more of the above referenced embodiments of the first aspect of the present invention having a complex viscosity of from about 1 to about 5, preferably from about 2 to about 5, and more preferably from about 2 to about 4 before crosslinking. In some embodiments, the solvent free, injectable, radiopaque composite network of the present invention includes any one or more of the above referenced embodiments of the first aspect of the present invention wherein the solvent free, injectable, radiopaque composite network is from about 20% to about 60%, preferably from about 30% to about 60%, and more preferably from about 40% to about 55% crosslinked after curing.

In one or more embodiments, the solvent free, injectable, radiopaque composite network of the present invention includes any one or more of the above referenced embodiments of the first aspect of the present invention having an average cure time of from about 30 min to about 70 min, preferably from about 30 min to about 60 min, and more preferably from about 30 min to about 50 min. In some embodiments, the solvent free, injectable, radiopaque composite network of the present invention includes any one or more of the above referenced embodiments of the first aspect of the present invention a time to gel point of from about 30 min to about 200 min, preferably from about 30 min to about 100 min, and more preferably from about 40 min to about 60 min at 25° C. In various embodiments, the solvent free, injectable, radiopaque composite network of the present invention includes any one or more of the above referenced embodiments of the first aspect of the present invention having a time to gel point of from about 10 min to about 70 min, preferably from about 15 min to about 60 min, and more preferably from about 30 min to about 50 min at 37° C.

In one or more embodiments, the solvent free, injectable, radiopaque composite network of the present invention includes any one or more of the above referenced embodiments of the first aspect of the present invention having a maximum compressive stress of from about 1 MPa to about 5 MPa, preferably from about 2 MPa to about 5 MPa, and more preferably from about 2 MPa to about 4 MPa after crosslinking. In various embodiments, the solvent free, injectable, radiopaque composite network of the present invention includes any one or more of the above referenced embodiments of the first aspect of the present invention having a maximum strain at break of from about 20 MPa to about 60 MPa, preferably from about 25 MPa to about 50 MPa, and more preferably from about 25 MPa to about 40 MPa after crosslinking.

In one or more embodiments, the solvent free, injectable, radiopaque composite network of the present invention includes any one or more of the above referenced embodiments of the first aspect of the present invention wherein the solvent free, injectable, radiopaque composite network is injectable prior to curing. In some embodiments, the solvent free, injectable, radiopaque composite network of the present invention includes any one or more of the above referenced embodiments of the first aspect of the present invention wherein the solvent free, injectable, radiopaque composite network has a compressive strength comparable to human vertebra. In one or more embodiments, the solvent free, injectable, radiopaque composite network of the present invention includes any one or more of the above referenced embodiments of the first aspect of the present invention wherein the radiopaque composite network is seeded with mesenchymal stem cells.

In a second aspect, the present invention is directed to a method for making the solvent free, injectable, radiopaque composite network described herein, comprising: preparing a poly(allyl glycidyl ether succinate) copolymer comprising one or more allyl functional groups and a residue of a mono, di, or multi-functional initiating alcohol; adding a plurality of radiopaque ceramics or metal salt molecules poly(allyl glycidyl ether succinate) copolymer and mixing to ensure even distribution of the plurality of radiopaque ceramics or metal salt molecules throughout the poly(allyl glycidyl ether succinate) copolymer; and adding a desired quantity of a multi-functional thiol crosslinker and allowing the mixture to crosslink to form the solvent free, injectable, radiopaque composite network described herein. In some of these embodiments, the radiopaque composite network is seeded with mesenchymal stem cells.

In some embodiments, the method for making the solvent free, injectable, radiopaque composite network of the present invention includes any one or more of the above referenced embodiments of the second aspect of the present invention wherein the poly(allyl glycidyl ether succinate) copolymer is the reaction product of succinic anhydride, allyl glycidyl ether, and a mono or di-functional initiating alcohol, and is formed by ring-opening polymerization using Mg(BHT)₂(THF)₂ as a catalyst. In one or more embodiments, the method for making the solvent free, injectable, radiopaque composite network of the present invention includes any one or more of the above referenced embodiments of the second aspect of the present invention wherein the poly(allyl glycidyl ether succinate) copolymer has a single poly(allyl glycidyl ether succinate) copolymer chain covalently bonded to the residue of a mono-functional initiating alcohol. In some other embodiments, the method for making the solvent free, injectable, radiopaque composite network of the present invention includes any one or more of the above referenced embodiments of the second aspect of the present invention wherein the poly(allyl glycidyl ether succinate) copolymer has two poly(allyl glycidyl ether succinate) copolymer chain covalently bonded to the residue of a di-functional initiating alcohol.

In one or more embodiments, the method for making the solvent free, injectable, radiopaque composite network of the present invention includes any one or more of the above referenced embodiments of the second aspect of the present invention wherein the mono-functional initiating alcohol is selected from the group consisting of propargyl alcohol, benzyl alcohol, allyl alcohol, 4-dibenzyocyclooctynol, 4-hydroxybutan-2-one, 3-hydroxypropan-2-one, 5-hydroxypentan-2-one, 6-hydroxyhexan-2-one, 7-hydroxyheptan-2-one, 8-hydroxyoctan-2-one, 5-norbornen-2-ol, α-bromoisobtyryl 4-methanol benzylmethanoate, and combinations thereof. In some embodiments, the method for making the solvent free, injectable, radiopaque composite network of the present invention includes any one or more of the above referenced embodiments of the second aspect of the present invention wherein the di-functional initiating alcohol is selected from the group consisting of 1,2-ethanediol, 1,3-propane diol, 1,4-butane diol, 1,5-pentane diol, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol, 1,19-nonadecanediol, 1,20-icosanediol, and combinations thereof.

In one or more embodiments, the method for making the solvent free, injectable, radiopaque composite network of the present invention includes any one or more of the above referenced embodiments of the second aspect of the present invention having the formula:

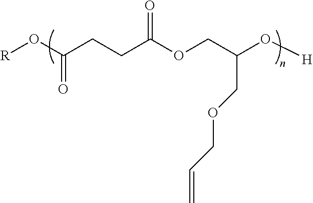

where n is an integer from about 10 to about 60 and R is a residue of an initiating alcohol. In some other embodiments, the method for making the solvent free, injectable, radiopaque composite network of the present invention includes any one or more of the above referenced embodiments of the second aspect of the present invention having the formula:

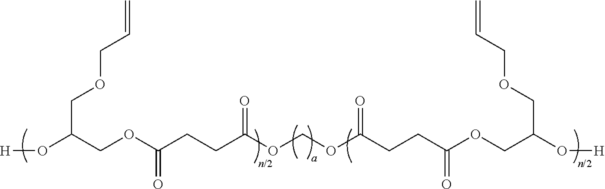

where n is an integer from about 10 to about 60 and where a is an integer from about 2 to about 12. In one or more embodiments, the method for making the solvent free, injectable, radiopaque composite network of the present invention includes any one or more of the above referenced embodiments of the second aspect of the present invention wherein the poly(allyl glycidyl ether succinate) copolymer has a degree of polymerization of from about 10 to about 60, preferably from about 25 to about 55, and more preferably from about 29 to about 50.

In one or more embodiments, the method for making the solvent free, injectable, radiopaque composite network of the present invention includes any one or more of the above referenced embodiments of the second aspect of the present invention wherein the multi-functional thiol crosslinking compound is selected from the group consisting of trimethylolpropane tris(3-mercaptopropionate), trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol), and combinations thereof. In some embodiments, the method for making the solvent free, injectable, radiopaque composite network of the present invention includes any one or more of the above referenced embodiments of the second aspect of the present invention wherein the multi-functional thiol crosslinking compound is trimethylolpropane tris(3-mercaptopropionate). In certain embodiments, the method for making the solvent free, injectable, radiopaque composite network of the present invention includes any one or more of the above referenced embodiments of the second aspect of the present invention wherein the multi-functional thiol crosslinking compound has the formula:

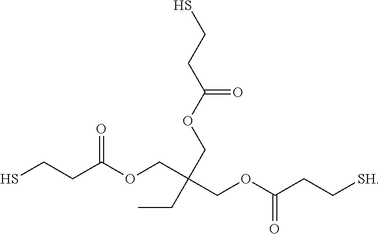 [SMILES: CCC(COC(=O)CCS)(COC(=O)CCS)COC(=O)CCS]

In one or more embodiments, the method for making the solvent free, injectable, radiopaque composite network of the present invention includes any one or more of the above referenced embodiments of the second aspect of the present invention having a consumption percentage of from about 30% to about 60%, preferably from about 35% to about 60%, and more preferably from about 40% to about 55% of the solvent free, injectable, radiopaque composite network.

In various embodiments, the method for making the solvent free, injectable, radiopaque composite network of the present invention includes any one or more of the above referenced embodiments of the second aspect of the present invention wherein the plurality of radiopaque ceramics or metal salt molecules comprise a radiopaque ceramic or metal salt of strontium, barium, calcium, or a combination thereof. In some embodiments, the method for making the solvent free, injectable, radiopaque composite network of the present invention includes any one or more of the above referenced embodiments of the second aspect of the present invention wherein the plurality of radiopaque ceramic molecules are selected from the group consisting of strontium carbonate, barium sulfate, α-alpha tricalcium phosphate (αTCP), and combinations thereof. In one or more embodiments, the method for making the solvent free, injectable, radiopaque composite network of the present invention includes any one or more of the above referenced embodiments of the second aspect of the present invention wherein the plurality of radiopaque ceramic molecules comprise strontium carbonate.

In one or more embodiments, the method for making the solvent free, injectable, radiopaque composite network of the present invention includes any one or more of the above referenced embodiments of the second aspect of the present invention wherein the plurality of radiopaque ceramics or metal salt molecules comprise from about 20 mol. % to about 90 mol. %, preferably from about 30 mol. % to about 80 mol. %, and more preferably from about 40 mol. % to about 60 mol. % of the solvent free, injectable, radiopaque composite network by mass of the solvent free, injectable, radiopaque composite network.

In one or more embodiments, the method for making the solvent free, injectable, radiopaque composite network of the present invention includes any one or more of the above referenced embodiments of the second aspect of the present invention having a complex viscosity of from about 1 to about 5, preferably from about 2 to about 5, and more preferably from about 2 to about 5% before crosslinking. In some embodiments, the method for making the solvent free, injectable, radiopaque composite network of the present invention includes any one or more of the above referenced embodiments of the second aspect of the present invention wherein the solvent free, injectable, radiopaque composite network is from about 20% to about 60%, preferably from about 30% to about 60%, and more preferably from about 30% to about 50% crosslinked after curing. In one or more embodiments, the method for making the solvent free, injectable, radiopaque composite network of the present invention includes any one or more of the above referenced embodiments of the second aspect of the present invention wherein the rate of crosslinking can be controlled by varying the concentration of the multifunctional thiol crosslinker, varying the DP of the poly(allyl glycidyl ether succinate) copolymer, and/or varying the temperature.

In a third aspect, the present invention is directed to a percutaneous polymeric cement for vertebral augmentation comprising the solvent free, injectable, radiopaque composite network described above. In one or more of these embodiments, the percutaneous polymeric cement is seeded with mesenchymal stem cells.

In a fourth aspect, the present invention is directed to a method for vertebral augmentation comprising: preparing a first component comprising a poly(allyl glycidyl ether succinate) copolymer comprising one or more allyl functional groups and a residue of a mono or di or multi-functional initiating alcohol, and a plurality of radiopaque ceramics or metal salt molecules; preparing a second component comprising a multifunctional thiol crosslinker; mixing the first and second component and waiting from about 10 to about 25 min; transferring the mixture into a syringe having a needle sized to be inserted into the damaged area of a vertebra; injecting the mixture into a damaged area of a vertebra and allowing the poly(allyl glycidyl ether succinate) copolymer to crosslink.

In one or more embodiments, the method for vertebral augmentation of the present invention includes any one or more of the above referenced embodiments of the fourth aspect of the present invention wherein the poly(allyl glycidyl ether succinate) copolymer has the formula:

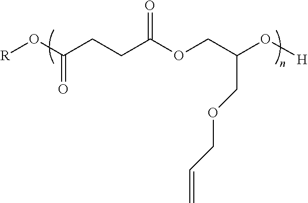

where n is an integer from about 10 to about 60 and R is a residue of an initiating alcohol. In some other embodiments, the method for vertebral augmentation of the present invention includes any one or more of the above referenced embodiments of the fourth aspect of the present invention wherein the poly(allyl glycidyl ether succinate) copolymer has the formula:

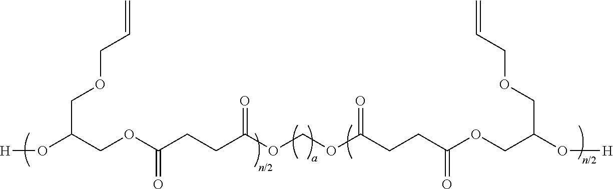

where n is an integer from about 10 to about 60 and where a is an integer from about 2 to about 12.

In one or more embodiments, the method for vertebral augmentation of the present invention includes any one or more of the above referenced embodiments of the fourth aspect of the present invention wherein the plurality of radiopaque ceramics of metal salt molecules are selected from the group consisting of strontium carbonate, barium sulfate, α-alpha tricalcium phosphate (αTCP), and combinations thereof. In some embodiments, the method for vertebral augmentation of the present invention includes any one or more of the above referenced embodiments of the fourth aspect of the present invention wherein the step of preparing a first component further comprises seeding the percutaneous polymeric cement with mesenchymal stem cells.

In one or more embodiments, the method for vertebral augmentation of the present invention includes any one or more of the above referenced embodiments of the fourth aspect of the present invention wherein the step of injecting further comprises creating an opening in the damaged vertebra sized to receive the mixture. In one or more embodiments, the method for vertebral augmentation of the present invention includes any one or more of the above referenced embodiments of the fourth aspect of the present invention wherein the step of creating an opening comprises forming a cavity in the vertebra body of the damaged vertebra using a drill or other cutting instrument.

In one or more embodiments, the method for vertebral augmentation of the present invention includes any one or more of the above referenced embodiments of the fourth aspect of the present invention wherein the rate of crosslinking in the step of crosslinking can be controlled by varying the concentration of crosslinker, the degree of polymerization of the PAGES copolymer, and/or the temperature. In some embodiments, the method for vertebral augmentation of the present invention includes any one or more of the above referenced embodiments of the fourth aspect of the present invention wherein the step of injecting and the step of allowing the poly(allyl glycidyl ether succinate) copolymer to crosslink are performed in vivo.

In a fifth aspect, the present invention is directed to a method for treating vertebral compression fractures using the method for vertebral augmentation described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

For a more complete understanding of the features and advantages of the present invention, reference is now made to the detailed description of the invention along with the accompanying figures in which:

FIGS. 1A-D are a schematic diagram and graphs showing polymer network formation through crosslinking of PAGES polymer. FIG. 1A is a schematic drawing showing injectable PAGES polymer and crosslinker mixture forming a covalent network through thiol-ene addition reactions. Specific crosslinking percentages can be targeted by adjusting the stoichiometric ratio of allyl groups in the polymer to thiol groups in the crosslinker. FIG. 1B shows Fourier Transform Infrared (FTIR) spectroscopy data of a 60% crosslinked sample demonstrating decrease in allyl stretch at 1650 cm⁻¹ as crosslinking occurs over time. FIG. 1C is a graph showing the evolution of normalized allyl stretch intensity at 1650 cm⁻¹ as crosslinking occurs over time for samples with varying targeted crosslinking (20%, 30%, and 60% of allyl groups reacted). FIG. 1D is a graph showing the change in normalized allyl stretch intensity at 1650 cm⁻¹ between 15 minutes and 1 day after mixing polymer with crosslinker for samples with varying targeted crosslinking (20%, 30%, and 60% of allyl groups reacted) (n=5). The box plots show median, first quartile, third quartile, maximum, and minimum. The extent of statistical significance was determined using Tukey's multiple comparisons test with *p<0.05, **p<0.01, ***p<0.001, ****p<0.0001.

FIGS. 2A-N are graphs showing the results of rheological investigation of gelation time for crosslinking PAGES samples in which, FIGS. 2A-B show the time to gelation point for PAGES DP₂₉ and DP₄₈ samples with different amounts of targeted crosslinking (20%, 30%, and 60% of allyl groups reacted) at room temperature (25° C.) (n=3) (FIG. 2A) and physiological temperature (37° C.) (n=3) (FIG. 2B); FIGS. 2C-N are graphs showing the median sample storage modulus and loss modulus at 10 rad/s as crosslinking occurs overtime for DP₂₉ (FIGS. 2C-H) and DP₄₈ (FIGS. 2I-N) PAGES samples with varying amounts of targeted crosslinking (20% (FIGS. 2C, F, I, L), 30% (FIGS. 2D, G, J, M), and 60% (FIGS. 2E, H, K, N) of allyl groups reacted) at 25° C. (FIGS. 2C-E, I-K) and 37° C. (FIGS. 2F-H, L-N). The bar charts show mean with error bars representing standard deviation. The extent of statistical significance was determined using Tukey's multiple comparisons test with *p<0.05, **p<0.01, ***p<0.001, ****p<0.0001.

FIGS. 3A-C are graphs showing the compressive strength of PAGES DP₂₉ and DP₄₈ samples with different amounts of targeted crosslinking (20%, 30%, and 60% of allyl groups reacted) allowed to cure under ambient conditions for varying amounts of time (1, 7, and 14 days) wherein FIG. 3A is a graph showing Median compressive stress-strain curves produced after samples were allowed to cure under ambient conditions for 1 day; FIG. 3B is a graph showing the maximum compressive stress at break for samples allowed to cure under ambient conditions for varying amounts of time (n=5); and FIG. 3C is a graph showing the strain at break for samples allowed to cure under ambient conditions for varying amounts of time (n=5). The box plots show median, first quartile, third quartile, maximum, and minimum. The extent of statistical significance was determined using Tukey's multiple comparisons test with *p<0.05, **p<0.01, ***p<0.001, ****p<0.0001.

FIGS. 4A-F are graphs showing the addition of ceramics increases compressive strength of polymer blocks and the compressive strength of PAGES DP₂₉ and DP₄₈ samples at 60% crosslinking with varying ceramic additives (alpha tricalcium phosphate (αTCP), strontium carbonate (SrCO₃), and barium sulfate (BaSO₄)) allowed to cure under ambient conditions for varying amounts of time (1, 7, 14 and 28 days) in which FIGS. 4A-B are graphs showing the median compressive stress-strain curves produced after PAGES DP₂₉ (FIG. 4A) and DP₄₈ (FIG. 4B) samples were allowed to cure under ambient conditions for 1 day; FIGS. 4C-D are graphs showing the maximum compressive stress at break for PAGES DP₂₉ (FIG. 4C) and DP₄₈ (FIG. 4D) samples allowed to cure under ambient conditions for varying amounts of time (n=5); and FIG. 4E-F are graphs showing strain at break for PAGES DP₂₉ (FIG. 4E) and DP₄₈ (FIG. 4F) samples allowed to cure for varying amounts of time (n=5). The box plots show median, first quartile, third quartile, maximum, and minimum. The extent of statistical significance was determined using Tukey's multiple comparisons test with *p<0.05, **p<0.01, ***p<0.001, ****p<0.0001.

FIGS. 5A-F are graphs regarding the retained mechanical strength exhibited by PAGES DP₂₉, 60% crosslinked materials while degrading in which FIGS. 5A-B are graphs showing representative compressive loading and unloading curves of PAGES samples without any ceramic additive (FIG. 5A) and with 50% strontium carbonate (SrCO₃) additive (FIG. 5B) after degrading in 1M HCl for varying amounts of time (6 hours, 1 week, 10 weeks); FIG. 5C is a graph showing the mass of PAGES samples without any ceramic additive, with 50% SrCO₃ additive, and with 50% BaSO₄ additive after degrading in PBS for up to 26 weeks (n=6); FIG. 5D is a graph showing compressive modulus of PAGES samples without any ceramic additive, with 50% SrCO₃ additive, and with 50% BaSO₄ additive after degrading in PBS for up to 26 weeks (n=6); FIG. 5E) is a graph showing the mass of PAGES samples without any ceramic additive, with 50 w % SrCO₃ additive, and with 50% BaSO₄ additive after degrading in 1M HCl for up to 26 weeks (n=6); and FIG. 5F) a graph showing the compressive modulus of PAGES samples without any ceramic additive, with 50% SrCO₃ additive, and with 50% BaSO₄ additive after degrading in 1M HCl for up to 26 weeks (n=6). The plotted data points show mean with error bars representing standard deviation.

FIGS. 6A-F are a series of images and a graph showing the results of an in vitro analysis of DP₂₉ PAGES, 60% crosslinked materials with ceramic additives in which FIGS. 6A-E are representative images of rat mesenchymal stem cells (MSCs) after 17 days of culture on either tissue culture plates or a thin layers of PAGES network with or without additives (alpha tricalcium phosphate (αTCP), strontium carbonate (SrCO₃), and barium sulfate (BASO₄)). All scale bars represent 50 m. Blue color represents cell nuclei stained using DAPI, and red color represents osteocalcin. FIG. 6F is a graph showing the quantification of percentage of cells expressing osteocalcin (n=9). Box plots show median, maximum, and minimum. The degree of statistical significance determined using Tukey's multiple comparisons test with *p<0.05, **p<0.01, ***p <0.001, ****p<0.0001.

FIGS. 7A-J images and graphs showing the use of DP₂₉ PAGES polymer at 60% crosslinking as a vertebral augmentation material in an ex vivo rabbit vertebral augmentation procedure in which FIGS. 7A-G are microcomputed tomography (CT) images of rabbit vertebra during ex vivo vertebral augmentation. All scale bars represent 3 mm. Solid arrows highlight the location of a drilled cavity. The hollow arrows represent porous bone tissue that can be filled with augmentation material. FIG. 7A) is a CT image of broken rabbit vertebra. FIG. 7B is a CT image of broken rabbit vertebra filled with PAGES with no additive. FIGS. 7C & F are CT image of rabbit vertebra filled with PAGES with 50% strontium carbonate (SrCO₃). FIG. 7D is a CT image of rabbit vertebra filled with PAGES with 50% SrCO₃ with teal color displaying infill of PAGES into the porous bone structure. FIGS. 7E and 7G are CT images of rabbit vertebra filled with poly(methyl methacrylate) (PMMA), the current clinical standard for vertebral augmentation. FIG. 7H is a graph showing the percentage of a drilled cavity in a rabbit vertebra that is filled with augmentation material using standard augmentation techniques (n=13 for PAGES+SrCO₃, n=6 for PMMA). FIG. 7I) is a graph showing averaged compressive stress-strain curves for rabbit vertebra initially, after being fractured, or after being fractured and filled with augmentation material (n=8, n=6 for PMMA). FIG. 7J is a graph showing the maximum compressive stress at break for rabbit vertebra initially, after being fractured, or after being fractured and filled with augmentation materials (n=8, n=6 for PMMA). The plotted data shows mean with error bars representing standard error of the mean. Box plots show median, first quartile, third quartile, maximum, and minimum. The degree of statistical significance determined using unpaired t-test or Tukey's multiple comparisons test with *p<0.05, **p<0.01, ***p<0.001, ****p<0.0001.

FIG. 8 is an annotated ¹H NMR (CDCl₃, 20° C., 600 MHz) of PAGES DP₂₉.

FIG. 9 is an annotated ¹H NMR spectra (CDCl₃, 20° C., 600 MHz) of PAGES DP₄₈.

FIG. 10 is a graph showing size exclusion chromatography (SEC) chromatograms (RI detector) of each PAGES polymer (DP₂₉ and DP₄₈) in THF (n=1). Molecular weight is calculated by MALS.

FIG. 11 is a deconvoluted size exclusion chromatography (SEC) chromatogram of PAGES DP₂₉ in THF (n=1). Molecular weight is calculated by MALS.

FIG. 12 is a deconvoluted size exclusion chromatography (SEC) chromatogram of PAGES DP₄₈ in THF (n=1). Molecular weight is calculated by MALS.

FIG. 13 is a thermogravimetric analysis (TGA) of each PAGES polymer (DP₂₉ and DP₄₈) used to determine degradation temperature (T_{d}) at 10% mass loss (n=1).

FIG. 14 is a graph showing differential scanning calorimetry (DSC) thermograms for the second heating cycle of PAGES polymers (DP₂₉ and DP₄₈) used to determine glass transition temperature (T_{g}) (n=1).

FIG. 15 is a rheological frequency sweep of each neat PAGES polymers (DP₂₉ and DP₄₈) at 25° C. (n=1).

FIGS. 16A-C are graphs showing gelation time comparison at 25° C. and 37° C. for 60% crosslinked PAGES DP₄₈ with and without the addition of 50% BaSO₄, in which FIG. 16A is a comparison of gelation time at 25° C. and 37° C. (n=3) (Error bars represent standard deviation);

FIG. 16B is a graph of representative storage and loss modulus evolution over time for 60% crosslinked PAGES DP₄₈ with and without the addition of 50% BaSO₄ at 25° C.; and FIG. 16C is a graph showing representative storage and loss modulus evolution over time for 60% crosslinked PAGES DP₄₈ with and without the addition of 50% BaSO₄ at 37° C.

FIGS. 17A-C are graphs showing gelation time comparison at 25° C. and 37° C. for 60% crosslinked PAGES DP₂₉ with and without the addition of 50% SrCO₃ in which FIG. 17A is a graph showing a comparison of gelation time at 25° C. and 37° C. (n=3). Error bars represent standard deviation; FIG. 17B is a graph of representative storage and loss modulus evolution over time for 60% crosslinked PAGES DP₂₉ with and without the addition of 50% SrCO₃ at 25° C.; and FIG. 17C is a graph of representative storage and loss modulus evolution over time for 60% crosslinked PAGES DP₂₉ with and without the addition of 50% SrCO₃ at 37° C.

FIGS. 18A-D is a collection of compressive stress-strain curves of PAGES DP₄₈ (FIGS. 18A-C) and DP₂₉ (FIG. 18D samples with different amounts of targeted crosslinking (20% (FIG. 18A), 30% (FIG. 18B), and 60% (FIGS. 18C-D) of allyl groups reacted) produced after samples were allowed to cure under ambient conditions for 1 day.

FIGS. 19A-B are graphs showing a Neo-Hookean fit of compressive stress strain curves. The original stress strain curve was used to determine the compressive modulus between 5-7% strain (FIG. 19A), while the Neo-Hookean fit was used to determine the shear modulus (G) over the entire strain range of the data (FIG. 19B).

FIG. 20 is an image showing different PAGES composite materials made using SrCO₃, αTCP, and BaSO₄. The αTCP does not appear to have the same degree of homogenization as the other two composite formulations. Scale bar represents 3 cm.

FIG. 21 is a collection of scanning electron microscopy with energy-dispersive X-ray spectroscopy (SEM-EDS) images of different elements on the surface of a PAGES+50% SrCO₃ sample. Scale bar represents 50 μm. SrCO₃ is evenly distributed throughout PAGES polymer blocks.

FIGS. 22A-D is a collection of compressive stress-strain curves of PAGES DP₂₉ samples with varying ceramic additives (alpha tricalcium phosphate (αTCP) (FIG. 22A), strontium carbonate (SrCO₃) (FIG. 22B-C), and barium sulfate (BaSO₄)) (FIG. 22D) at 20 wt. % (FIGS. 22A, C) and 50 wt. % (FIGS. 22B, D) produced after samples were allowed to cure under ambient conditions for 1 day.

FIGS. 23A-B are a collection of compressive stress-strain curves of PAGES DP₄₈ samples with varying ceramic additives (alpha tricalcium phosphate (αTCP) (FIG. 23A), strontium carbonate (SrCO₃) (FIG. 23B) produced after samples were allowed to cure under ambient conditions for 1 day.

FIGS. 24A-D are graphs showing a Neo-Hookean fit of compressive stress strain curves. The original stress strain curve was used to determine the compressive modulus between 5-7% strain (FIGS. 24A,C), while the Neo-Hookean fit was used to determine the shear modulus (G) over the entire strain range of the data (FIGS. 24B,D).

FIGS. 25A-B are graphs of Rheology data showing variable gelation time of degree of polymerization of 48 (DP₄₈) single arm PAGES based on crosslinker, trimethylolpropane tris(3-mercaptopropionate), ratio (expressed as percentage of terminal alkenes reacted) and temperature. FIG. 25A shows the time to gel point for difference conditions. N=3, *: p<0.05, **p<0.01, ***p<0.001. FIG. 25B shows the averaged tan(6) curves for variable crosslink concentration and reaction temperature. Filled circle=25C and open circle=37C. Error bars represent standard deviation. As expected, the polymer forms a gel faster at higher crosslinking ratios; furthermore, the reaction proceeds faster at higher temperature. This data shows that the PAGES polymer has a clinically relevant working time for augmentation applications and that the material cures faster at body temperature.

FIGS. 26A-B are graphs showing cure times for different length PAGES polymers, DPₙ range of 6 to 55, at 60% of alkenes reacted with crosslinker in which FIG. 26A is a graph showing cure time vs DPₙ. and FIG. 26B is a graph showing cure time vs polymer complex viscosity at 10 rad/s. The cure times show that at low DP the polymer takes longer to cure, likely due to the shorter polymer chains having to reach a higher extent of reaction to form a percolating network. At higher DPₙ, viscosity appears to play a larger role in gelation rate as seen by the faster gelation time for the DP₅₄ polymer, which has lower viscosity, than the DP₄₈ polymer and DP₅₅ polymer. N=3, triangle=2-arm initiator, x=1-arm initiator, error bars=standard error.

FIGS. 27A-B are graphs showing compressive strength of DP₄₈ single arm PAGES polymers at different crosslinking ratios over 14 days in which FIG. 27A is a graph showing compressive stress at break in kPa. 60% crosslinked samples are significantly stronger than 20% crosslinked samples at Day 1. 20% and 30% crosslinked samples do exhibit increased peak compressive stress at Day 7 compared to Day 1 and FIG. 27B is a graph showing compressive strain at break. N=5 (N=4 for 30% Day 14 sample), *:p<0.05, **p:<0.01. 60% crosslinked samples exhibit stiffening over time with a significantly decreased strain at break without decreased peak compressive stress. These observations demonstrates that the polymers continue to evolve over time, presumably due to ongoing crosslinking causing an increase in network density. The peak material strength is at the low end of reported ultimate compressive stress of human vertebrae (1-6 MPa).

FIGS. 28A-B are graphs showing the addition of ceramics increases compressive strength and stiffness of polymer blocks. All samples were prepared with DP₄₈ single arm PAGES. FIG. 28A is a graph showing maximum compressive stress observed expressed in kPa. Addition of SrCO₃ increases compressive strength at all time points compared to no additive control. Addition of αTCP did not significantly increase compressive strength compared to control. FIG. 28B) is a graph showing compressive strain at break. Addition of either αTCP or SrCO₃ significantly decreased strain at break indicating increased stiffness compared to no additive control. All conditions demonstrate stiffening over time with significantly decreased strain at break. N=6, *:p<0.05, **p<0.01, ***p<0.001. Polymer block compressive strength is in the middle of the compressive strength range of a vertebral body following the addition of SrCO₃. SrCO₃ also increases the radiodensity of the resulting material, making it visible on fluoroscopy and so allowing for use in a radiographically guided application such as kyphoplasty or vertebroplasty.

FIGS. 29A-C are MicroCT images of PAGES filled into open-cell foam. Polymer was injected onto the superior surface of the foam and allowed to flow through the foam during the gelation process in which FIG. 29A is a single CT slice of PAGES with no additive filled foam through the y axis where an open arrow head represents an open cell foam, a solid arrow head represents polymer and FIG. 29B is a single CT slice of PAGES with 50% w/w SrCO₃ filled foam through the y axis (open arrow head=open cell foam, solid arrow head=polymer). The addition of strontium significantly increases polymer radiodensity, as expected. FIG. 29C is a 3D reconstruction showing the open cell foam in green and the filled polymer in pink.

### DETAILED DESCRIPTION OF THE ILLUSTRATIVE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

As set forth above, vertebral body compression fractures are a major cause of chronic back pain, particularly in older adults. The current standard of care is a percutaneous polymeric cement injection into the fractured vertebra of these patients to alleviate pain and stabilize the fracture. Augmentation is currently performed by injecting a polymethyl methacrylate (PMMA) slurry of polymer, monomer and initiator mixed with barium sulfate (BaSO₄) into the vertebrae, which then polymerizes in vivo. PMMA is nondegradable, possesses a 100-fold higher ultimate compressive strength (~100 MPa) than native vertebra (~1 MPa), and the in vivo free radical polymerization is highly exothermic. These factors are thought to contribute to the development of fractures in vertebra bodies above and/or below the augmented level during long-term recovery.

In various embodiments, the present invention is directed to a solvent-free poly(allyl glycidyl ether succinate) (PAGES) composite network for vertebral augmentation. These PAGES polymers in these composite networks crosslink in situ through thiol-ene click chemistry with a cure time at 37° C. ranging from 17 to 53 minutes based on degree of polymerization and crosslinker concentration. In various embodiments, the present invention is directed to poly(allyl glycidyl ether succinate) (PAGES)-based composite networks and their use as a percutaneous polymeric cement for vertebral augmentation. Advantageously, these PAGES-based copolymer composite networks have been found to have mechanical properties near that of native vertebra and should not, therefore, contribute to longer term complications due to the mechanical mismatch common to PMMA-based systems. These composite networks contain PAGES copolymers that are crosslinked with a multi-functional thiol crosslinker, such as trimethylolpropane tris(3-mercaptopropionate), via a thiol-ene addition mechanism, and contain one or more ceramic additives that make the networks radiopaque. These PAGES copolymers possess a low neat viscosity that scales with the degree of polymerization, affording injectability without the addition of solvents or diluents. The rate of crosslinking can be controlled via the concentration of the crosslinker and the temperature. In one or more embodiments, the composite networks may contain from about 30% to about 80% radiopaque materials by mass, without substantially affecting its crosslinking mechanism or set time and are not endothermic, thereby avoiding the risk of thermal damage to surrounding tissues. In various embodiments, the ceramic additives also assist with the proliferation and differentiation of any mesenchymal stem cells that are seeded onto the surface of the PAGES-based composite. Additionally, the thiol-ene “click” crosslinking reaction are known to be cell compatible, does not generate residual by-products, and has been applied in many other biomaterial applications.

The following terms may have meanings ascribed to them below, unless specified otherwise. As used herein, the terms “comprising” “to comprise” and the like do not exclude the presence of further elements or steps in addition to those listed in a claim. Similarly, the terms “a,” “an” or “the” before an element or feature does not exclude the presence of a plurality of these elements or features, unless the context clearly dictates otherwise. Further, the term “means” used many times in a claim does not exclude the possibility that two or more of these means are actuated through a single element or component.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term “about.”

It should be also understood that the ranges provided herein are a shorthand for all of the values within the range and, further, that the individual range values presented herein can be combined to form additional non-disclosed ranges. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “functionalized initiating alcohol” and “functional initiating alcohol” are is used herein interchangeably to refer to an alcohol molecule capable of initiating ring opening copolymerization of a succinic anhydride monomer and an allyl glycidyl ether monomer, in the presence of Mg(BHT)₂(THF)₂ catalyst. A functionalized initiating alcohol having a single hydroxyl group capable of initiating ring opening copolymerization of a succinic anhydride monomer and an allyl glycidyl ether monomer, in the presence of Mg(BHT)₂(THF)₂ catalyst will be referred to herein interchangeably as a “mono-functional initiating alcohol,” “mono functional initiating alcohol,” “mono-functionalized initiating alcohol initiating alcohol” or “mono functionalized initiating alcohol initiating alcohol.” As follows, the terms “residue of the mono-functionalized initiating alcohol” or “mono-functionalized initiating alcohol residue” and the like, refer to the parts of the mono-functionalized initiating alcohol that remain bound at the end of the PAGES copolymer chain after it initiates polymerization.

Similarly, the terms “bifunctional alcohol initiator” “bifunctionalized alcohol initiator,” “bi-functional alcohol initiator,” and “bi-functionalized alcohol initiator” are used interchangeably herein to refer to a functionalized initiating alcohol having two active hydroxyl groups, each capable of initiating ring opening copolymerization of a succinic anhydride monomer and an allyl glycidyl ether monomer, in the presence of Mg(BHT)₂(THF)₂ catalyst. As follows, the terms “residue of the bi-functionalized initiating alcohol” or “bi-functionalized initiating alcohol residue” and the like, refer to the parts of the mono-functionalized initiating alcohol that remain bound at the end of the PAGES copolymer chain after it initiates polymerization.

Similarly, the terms “multifunctional alcohol initiator,” “multi-functional alcohol initiator,” “multifunctionalized alcohol initiator,” “multi-functionalized alcohol initiator” are used interchangeably to refer to a functional initiating alcohol having three or more active hydroxyl groups, each capable of initiating ring opening copolymerization of a succinic anhydride monomer and an allyl glycidyl ether monomer, in the presence of Mg(BHT)₂(THF)₂ catalyst. As follows, the terms “residue of the multi-functionalized initiating alcohol” or “bi-functionalized initiating alcohol residue” and the like, refer to the parts of the mono-functionalized initiating alcohol that remain bound at the end of the PAGES copolymer chain after it initiates polymerization.

Generally, a “ring opening polymerization” reaction is defined as a reaction in which two or more cyclic monomers repeatedly react with a terminal reactive center, thus opening their rings during the successive polymerization. Similarly, the term “ring opening copolymerization” is used to refer to a ring opening polymerization having two or more different cyclic monomers.

As used herein, the “degree of polymerization” (DP) generally refers to the number of monomer repeat units present in a given polymer chain. As applied to polymer or copolymer having two or more arms extending from a central core, such as a star polymer, the DP will be the number of monomer repeat units in each arm extending from a central core.

As used herein, the term “crosslinker” refers to a molecule that is capable of reacting with two or more polymer chains in order to link them together.

As used herein to apply to a polymer, copolymer or resin, the term “neat” is used herein to refer to a polymer without solvent or diluent.

As used herein, the terms “click reaction,” “click chemistry,” “click chemistry methods,” and “click chemistry reactions,” are used interchangeably to refer to a group of orthogonal conjugation reactions, generally referred to in the art as “click” reactions, that fulfill the following prerequisites: (i) high yield, nearly quantitative conversion; (ii) biologically benign conditions (aqueous solution, ambient temperature, and near physiologic pH); (iii) limited or no residual byproduct. These reactions are typically simple to perform, high yielding, stereospecific, wide in scope, create only byproducts that can be removed without chromatography, and can be conducted in easily removable or benign solvents. Similarly, the term “clickable” refers to a molecule or functional group capable of bonding via a click reaction.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning, unless otherwise indicated.

Further, any compositions or methods provided herein can be combined with one or more of any of the other compositions and methods provided herein. The fact that given features, elements or components are cited in different dependent claims does not exclude that at least some of these features, elements or components maybe used in combination together.

In a first aspect, the present invention is directed to a solvent free, injectable, radiopaque composite network comprising: a poly(allyl glycidyl ether succinate) copolymer comprising one or more allyl functional groups and a residue of a mono, di, or multi-functional initiating alcohol; a multi-functional thiol crosslinking compound; and a plurality of radiopaque ceramics or metal salt molecules.

In various embodiments, the PAGES copolymers used for the solvent free resins of the present invention are formed by ring opening copolymerization of allyl glycidyl ether and succinic anhydride, initiated by a mono-, bi-, or multi-functional initiating alcohol and using a Mg(BHT)₂(THF)₂ catalyst, as set forth above and described in more detail below. The mono, di, or multi-functional initiating alcohol used to form the PAGES copolymers is not particularly limited, provided that it has one or more available hydroxyl functional groups and is capable of initiating ROCOP of succinic anhydride with allyl glycidyl ether in the presence of Mg(BHT)₂(THF)₂ or another magnesium catalyst. As will be apparent, the central core structure of these linear or star-shaped PAGES copolymers will comprise the residue of the mono, bi-, or multi-functional alcohol used to as the initiator, and the number of arms on these polymers is likewise controlled by the number of alcohol (i.e. hydroxyl) functional groups present on the multi-functional alcohol initiator. As used herein, the term “residue(s)” is used to refer generally to the portion of a monomer or other chemical unit that has been incorporated into a polymer or other large molecule. The PAGES copolymers of the present invention may, therefore, be linear or star shaped, depending upon they type of initiating alcohol used to form the polymer. In either case, the residue of the initiating alcohol used to form the copolymer will form the core structure from which the PAGES copolymer chains will extend.

In embodiments where the PAGES copolymers are to have a generally linear structure, a mono-functional or bi-functional alcohol initiator will be used. In embodiments where a mono-functional alcohol initiator is used to initiate the ring opening copolymerization reaction and the resulting polymers will have a single PAGES chain emanating outward from the residue of the initiating alcohol, which will be located on one end of the polymer chain. Suitable mono-functional alcohol initiators are not particularly limited and may include, without limitation, propargyl alcohol, benzyl alcohol, allyl alcohol, 4-dibenzyocyclooctynol, 4-hydroxybutan-2-one, 3-hydroxypropan-2-one, 5-hydroxypentan-2-one, 6-hydroxyhexan-2-one, 7-hydroxyheptan-2-one, 8-hydroxyoctan-2-one, 5-norbornen-2-ol, α-bromoisobtyryl 4-methanol benzylmethanoate or a combination thereof. In some embodiments, the mono-functional alcohol initiator is propargyl alcohol.

In some embodiments, the solvent free, injectable, radiopaque composite network will have the formula:

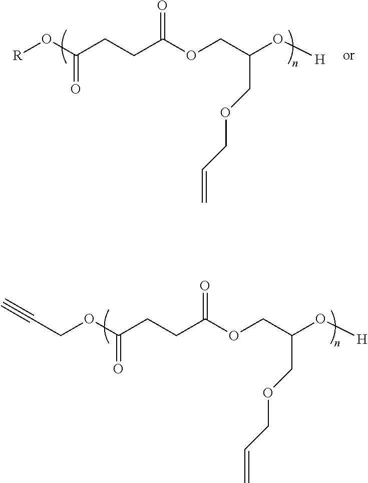

where n is an integer from about 10 to about 60 and R is a residue of an initiating alcohol.

In some embodiments, the initiating alcohol is di-functional and the resulting poly(allyl glycidyl ether succinate) copolymer will have two poly(allyl glycidyl ether succinate) copolymer chain covalently bonded to the residue of the di-functional initiating alcohol. In some embodiments, suitable bi-functional alcohol initiators may include, without limitation, 1,2-ethanediol, 1,3-propane diol, 1,4-butane diol, 1,5-pentane diol, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol, 1,19-nonadecanediol, 1,20-icosanediol, and combinations thereof. In one or more embodiments, the shaped poly(allyl glycidyl ether succinate) copolymer used to form the composite network of the present invention will be a linear copolymer having two poly(allyl glycidyl ether succinate) chains emanating from a residue of a bi-functional alcohol initiator.

In various embodiments, the poly(allyl glycidyl ether succinate) copolymer is a linear copolymer having the formula:

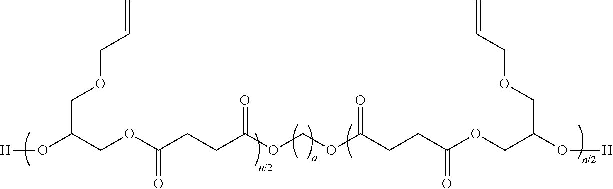

where n is an integer from about 10 to about 60 and where a is an integer from about 2 to about 12. In some of these embodiments, the poly(allyl glycidyl ether succinate) copolymer will be a linear copolymer having a formula selected from:

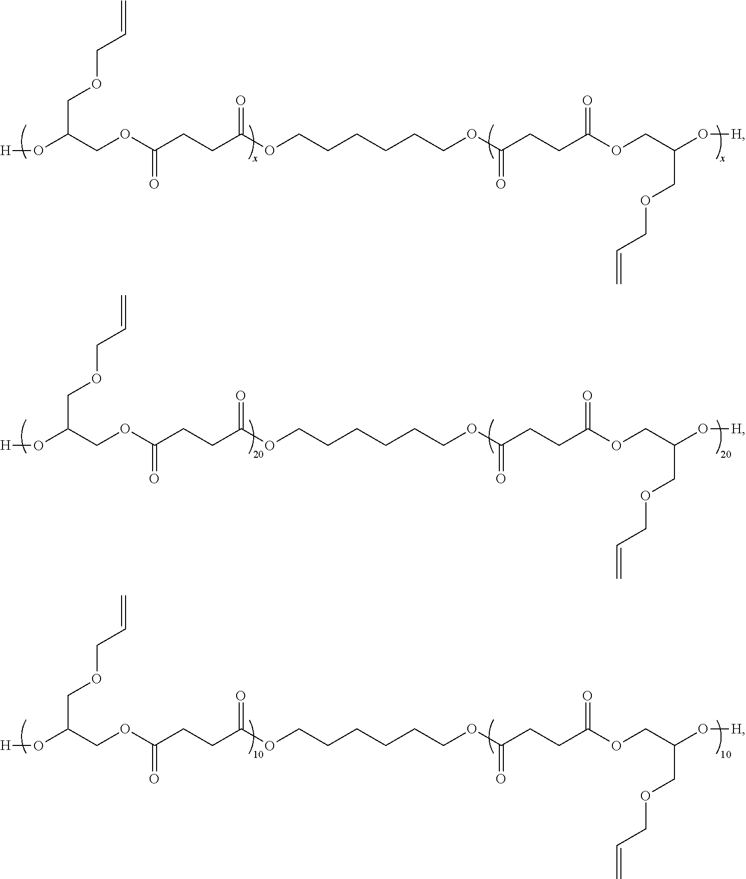

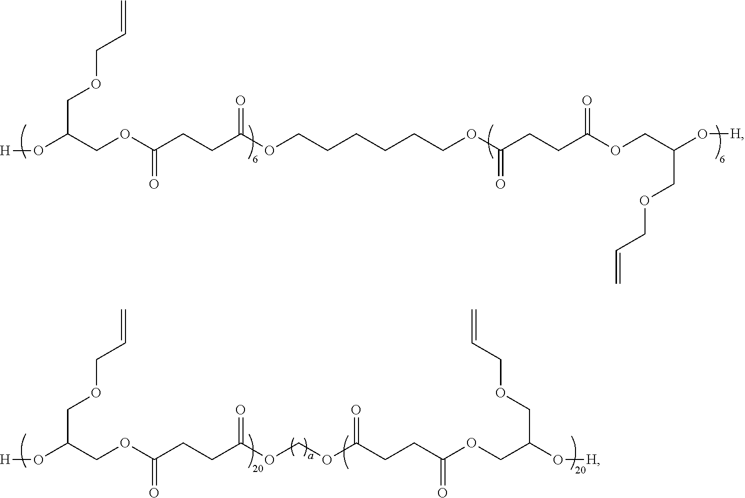

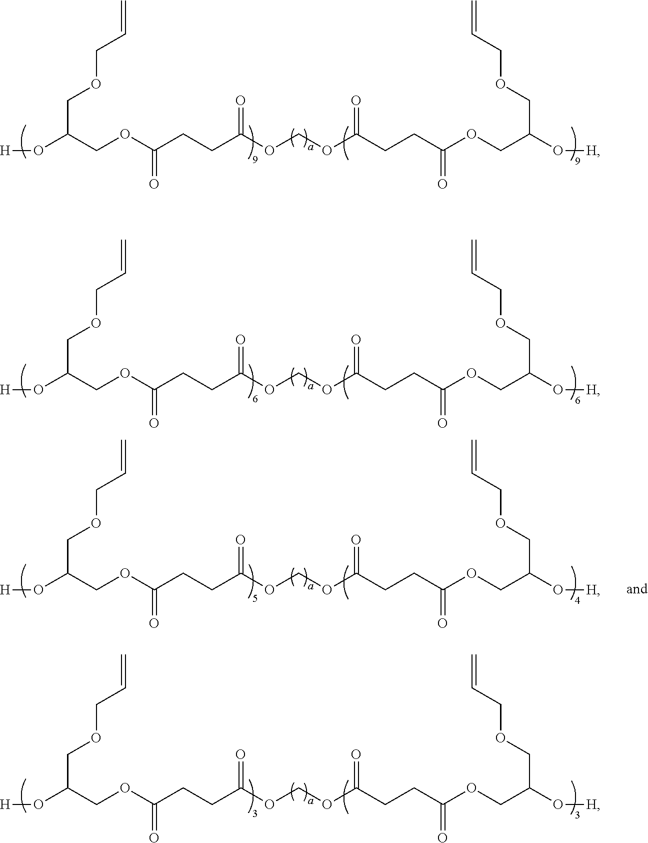

where x is an integer from about 2 to about 20.

In one or more embodiments, a multi-functional initiating alcohol may be used, forming a star-shaped polymer. As will be apparent, the central core structure in these copolymers will comprise the residue of the multi-functional alcohol initiators used to form them. By way of example, the terms “3-arm star” and “3-arm star-shaped” refer to PAGES copolymers with 3 arms derived from the ROCOP of succinic anhydride and allyl glycidyl ether initiated by an alcohol having three available hydroxyl groups, such as glycerin, 2-hydroxymethyl-1,3-propanediol, 1,1,1-tris(hydroxymethyl)ethane, or 1,1,1-tris(hydroxymethyl) propane, with three hydroxyl groups initiating ROCOP of one arm of the 3-arm star polymer and the remainder of the alcohol molecule forming the core. Similarly, in one or more embodiments, the terms “4-arm star” and “4-arm star-shaped” are used to refer to PAGES copolymers with 4 arms derived from the ROCOP of succinic anhydride and allyl glycidyl ether initiated by an alcohol having four available hydroxyl groups, such as meso-erythritol, pentaerythritol, or (2R,3R)-butane-1,2,3,4-tetrol, with each of the four hydroxyl groups initiating ROCOP of one arm of the 4-arm star polymer and the remainder of the alcohol molecule forming the core. As follows, the terms “5-arm star” and “5-arm star-shaped” refer to PAGES copolymers with 5 arms derived from the ROCOP of succinic anhydride and allyl glycidyl ether initiated by an alcohol having five available hydroxyl groups, such as adonitol, arabitol, (2R,3r,4S)-pentane-1,2,3,4,5-pentol, or fucitol, with five hydroxyl groups initiating ROCOP of one arm of the 5-arm star polymer and the remainder of the alcohol molecule forming the core. Similarly, the terms “6-arm star” and “6-arm star-shaped” refer to PAGES copolymers with 6 arms derived from the ROCOP of succinic anhydride and allyl glycidyl ether initiated by an alcohol having six available hydroxyl groups, such as hexakis[p-(hydroxymethyl)phenoxy]cyclotriphosphazene or dipentaerithyriol (dipenta), with six hydroxyl groups initiating ROCOP of one arm of the 6-arm star polymer and the remainder of the alcohol molecule forming the core. hexakis[p-(hydroxymethyl)phenoxy]cyclotriphosphazene or dipentaerithyriol (dipenta).

The PAGES copolymer used will have a complex viscosity of from about 30 Pa s to about 80 Pa s, preferably from about 35 Pa s to about 80 Pa s, and more preferably from about 35 Pa s to about 60 Pa s before crosslinking.

In various embodiments, the PAGES copolymer used will have a degree of polymerization of from about 10 to about 60, preferably from about 25 to about 55, and more preferably from about 29 to about 50.

As set forth above, the PAGES-based composite network of the present invention further comprises a di or multi-functional thiol crosslinking compound. The bi- or multi-functional thiol crosslinker is not particularly limited and any molecule having two or more available thiol groups may be used provided that the compound does not otherwise interfere with the reaction or reagents being used. In some embodiments, the bi- or multi-functional thiol crosslinker may be trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol) or a combination thereof. In some of these embodiments, the bi- or multi-functional thiol crosslinker is trimethylolpropane tris (3-mercaptopropionate). In some of these embodiments, the multi-functional thiol crosslinker has the formula:

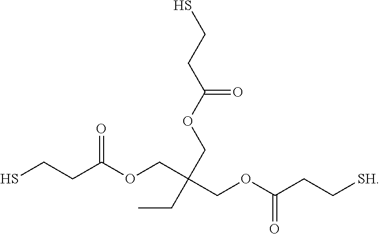 [SMILES: CCC(COC(=O)CCS)(COC(=O)CCS)COC(=O)CCS]

The bi- or multi-functional thiol crosslinker is not particularly limited and any molecule having two or more available thiol groups may be used provided that the compound does not otherwise interfere with the reaction or reagents being used. In some embodiments, the bi- or multi-functional thiol crosslinker may be trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol) or a combination thereof. In some of these embodiments, the bi- or multi-functional thiol crosslinker is trimethylolpropane tris (3-mercaptopropionate). In some embodiments, the bi- or multi-functional thiol crosslinker will have the formula:

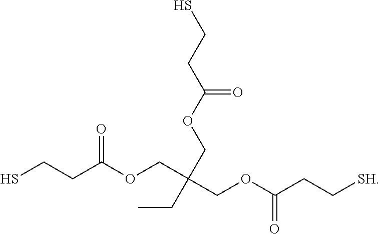 [SMILES: CCC(COC(=O)CCS)(COC(=O)CCS)COC(=O)CCS]

As set forth above, the composite networks of the present invention will also include a plurality of radiopaque ceramics or metal salt molecules. These radiopaque molecules permit better imaging of the composite networks and allow the material to be easily identified using fluoroscopic guidance during the augmentation procedure. In some embodiments, the radiopaque ceramics or metal salt molecules will comprise a radiopaque ceramic or metal salt of strontium, barium, calcium, or a combination thereof. In some embodiments, the radiopaque ceramic molecules are selected from the group consisting of strontium carbonate, barium sulfate, α-alpha tricalcium phosphate, and combinations thereof. In some embodiments, the radiopaque ceramic or metal salt molecules will comprise strontium carbonate.

In various embodiments, the plurality of radiopaque ceramics or metal salt molecules comprise from about 20% to about 90%, preferably from about 30% to about 80%, and more preferably from about mol. 20 to about 60 by mass of the radiopaque composite network.

As set forth above, the amount of bi- or multi-functional thiol crosslinker used will depend upon both the crosslink ratio used as well as the molecular weight of the thiol crosslinker. As will be appreciated, the percentage of available allyl groups on the PAGES copolymer that are crosslinked by the thiol crosslinker can be referred to as the “consumption percentage” or “percent consumption.” In various embodiments, the percent consumption for the composite networks of the present invention will be from about 30% to about 60%. In some embodiments, the consumption percentage for the composite networks of the present invention will be from about 35% to about 60%, in other embodiments, from about 40% to about 60%, in other embodiments, from about 45% to about 60%, in other embodiments, from about 50% to about 60%, in other embodiments, from about 55% to about 60%, in other embodiments, from about 30% to about 55%, in other embodiments, from about 30% to about 50%, in other embodiments, from about 30% to about 45%, in other embodiments, from about 30% to about 40%, and in other embodiments, from about 30% to about 35% of the polymer composite networks of the present invention.

In one or more embodiments, the radiopaque composite network will be from about 20% to about 60%, preferably from about 30% to about 50%, and more preferably from about 30% to about 40% crosslinked after fully curing.

In one or more embodiments, the radiopaque composite network will have an average cure time of from about 30 min to about 70 min, preferably from about 30 min to about 60 min, and more preferably from about 30 min to about 50 min.

In one or more embodiments, the radiopaque composite network will have a time to gel point of from about 30 min to about 200 min, preferably from about 30 min to about 100 min, and more preferably from about 40 min to about 60 min at 25° C.

In one or more embodiments, the radiopaque composite network will have a time to gel point of from about 10 min to about 70 min, preferably from about 15 min to about 60 min, and more preferably from about 20 min to about 50 min at 37° C.

In one or more embodiments, the radiopaque composite network will have a maximum compressive stress of from about 1 MPa to about 5 MPa, preferably from about 2 to about 5, and more preferably from about 2 to about 4 after crosslinking.

In some embodiments, the radiopaque composite network will have a maximum strain at break of from about 20 MPa to about 60 MPa, preferably from about 25 to about 50, and more preferably from about 25 to about 40 after crosslinking. In one or more of these embodiments, the solvent free, injectable, radiopaque composite network of the present invention is injectable prior to curing.

In various embodiments, the solvent free, injectable, radiopaque composite network of the present invention has a compressive strength that is comparable to human vertebra.

In still other embodiments, the solvent free, injectable, radiopaque composite network will be seeded with mesenchymal stem cells.

In a second aspect, the present invention is directed to a method for making the solvent free, injectable, radiopaque composite network described above, comprising: preparing a poly(allyl glycidyl ether succinate) copolymer comprising one or more allyl functional groups and a residue of a mono, di, or multi-functional initiating alcohol; adding a plurality of radiopaque ceramics or metal salt molecules poly(allyl glycidyl ether succinate) copolymer and mixing to ensure even distribution of said plurality of radiopaque ceramics or metal salt molecules throughout said poly(allyl glycidyl ether succinate) copolymer; and adding a desired quantity of a multi-functional thiol crosslinker and allowing the mixture to crosslink to form the solvent free, injectable, radiopaque composite network described above.

As set forth above, the poly(allyl glycidyl ether succinate) copolymer is the reaction product of succinic anhydride, allyl glycidyl ether, and a mono, di or multi-functional initiating alcohol, and is formed by ring-opening polymerization using Mg(BHT)₂(THF)₂ as a catalyst. Accordingly, these poly(allyl glycidyl ether succinate) copolymers will comprise one or more poly(allyl glycidyl ether succinate) polymer chains with allyl functional groups extending from the residue of the mono, di, or multi-functional initiating alcohol used to form them. These polymers will be linear or star shaped depending upon the type of initiating alcohol used, with mono or di-functional alcohol initiators forming generally linear PAGES copolymers and multi-functional alcohol initiators forming generally star shaped PAGES copolymers.

In some embodiments, the poly(allyl glycidyl ether succinate) copolymer has a single poly(allyl glycidyl ether succinate) copolymer chain covalently bonded to the residue of a mono-functional initiating alcohol. As set forth above, suitable the mono-functional initiating alcohols for this purpose may include, without limitation, propargyl alcohol, benzyl alcohol, allyl alcohol, 4-dibenzyocyclooctynol, 4-hydroxybutan-2-one, 3-hydroxypropan-2-one, 5-hydroxypentan-2-one, 6-hydroxyhexan-2-one, 7-hydroxyheptan-2-one, 8-hydroxyoctan-2-one, 5-norbornen-2-ol, α-bromoisobtyryl 4-methanol benzylmethanoate, and combinations thereof. In some embodiments the poly(allyl glycidyl ether succinate) copolymer may be formed as shown in Scheme 1, below.

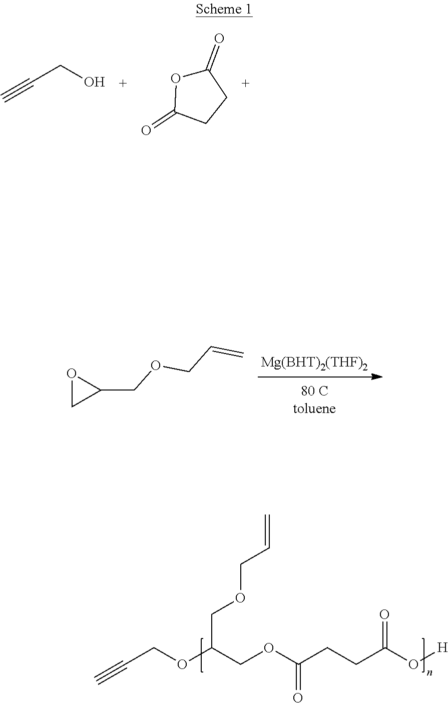

As set forth above, in some embodiments the PAGES copolymer used to form the solvent free, injectable, radiopaque composite network of the present invention will have the formula:

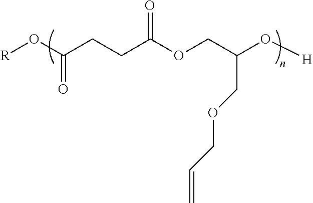

where n is an integer from about 10 to about 60 and R is a residue of an initiating alcohol.

In some other embodiments, a di-functional initiating alcohol is used. In these embodiments, the poly(allyl glycidyl ether succinate) copolymer will be generally linear, having two poly(allyl glycidyl ether succinate) copolymer chains covalently bonded to the residue of the di-functional initiating alcohol. As set forth above, suitable di-functional initiating alcohol may include, without limitation, 1,2-ethanediol, 1,3-propane diol, 1,4-butane diol, 1,5-pentane diol, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol, 1,19-nonadecanediol, 1,20-icosanediol, and combinations thereof.

In some embodiments the poly(allyl glycidyl ether succinate) copolymer may be formed as shown in Scheme 2, below.

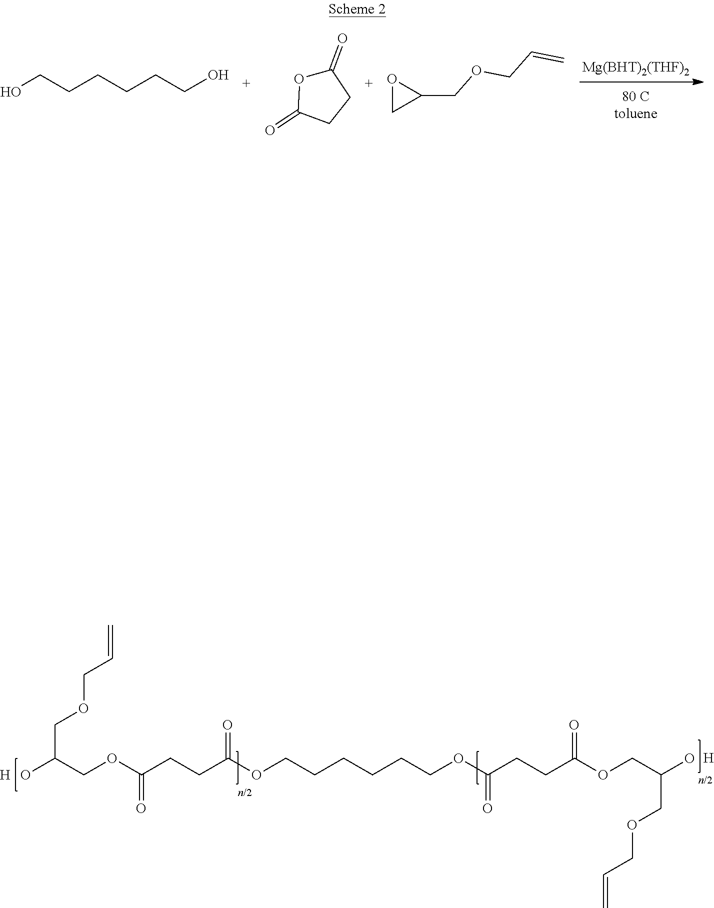

In one or more of these embodiments, the PAGES copolymer used to form the solvent free, injectable, radiopaque composite network of the present invention will have the formula:

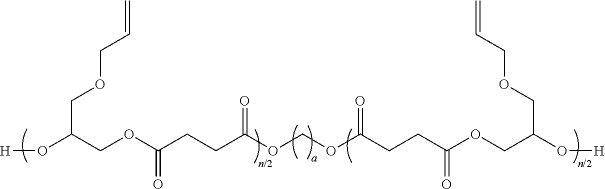

where n is an integer from about 10 to about 60 and where a is an integer from about 2 to about 12.

In various embodiments, the poly(allyl glycidyl ether succinate) copolymer used to form the polymer composite networks of the present invention will have a degree of polymerization of from about 10 to about 60, preferably from about 25 to about 55, and more preferably from about 29 to about 50.

In some embodiments, the poly(allyl glycidyl ether succinate) copolymer used to form the polymer composite networks of the present invention will have a complex viscosity of from about 30 Pa s to about 80 Pa s, preferably from about 35 Pa s to about 80 Pa s, and more preferably from about 35 Pa s to about 60 Pa s before the crosslinking step.

In the next step of the method, a quantity of radiopaque ceramics or metal salt molecules and added to the PAGES copolymers described above. The radiopaque ceramics or metal salt molecules used in the method are not particularly limited provided that they are radiopaque and that the compound does not otherwise interfere with the reaction or reagents being used”. In some embodiments, the radiopaque ceramics or metal salt molecules will be radiopaque ceramic or metal salts of strontium, barium, calcium, or a combination thereof. In some embodiments, the radiopaque ceramics or metal salt molecules may be strontium carbonate, barium sulfate, α-alpha tricalcium phosphate (αTCP), and combinations thereof. In some of these embodiments, the radiopaque ceramics or metal salt molecules will comprise strontium carbonate.

In various embodiments, the radiopaque ceramics or metal salt molecules comprise from about 20% to about 90%, preferably from about 30% to about 80%, and more preferably from about 30% to about 60% by mass of said solvent free, injectable, radiopaque composite network. The radiopaque ceramics or metal salt molecules may be mixed into the PAGES copolymers by any conventional means. While not absolutely required, it preferred that the radiopaque ceramics or metal salt molecules be evenly distributed throughout the PAGES copolymers prior to the crosslinking step.

Finally, a di- or multi-functional thiol crosslinking compound as described above is selected and mixed into the PAGES/radiopaque ceramics or metal salt molecules mixture. In various embodiments, the multi-functional thiol crosslinking compound is selected from the group consisting of trimethylolpropane tris(3-mercaptopropionate), trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol), and combinations thereof. In some of these embodiments, the multi-functional thiol crosslinking compound is trimethylolpropane tris(3-mercaptopropionate). In some of these embodiments, the multi-functional thiol crosslinking compound has the formula:

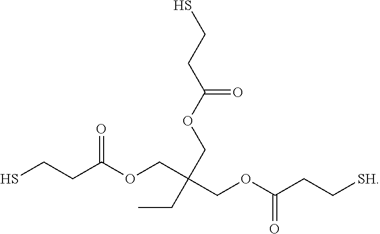 [SMILES: CCC(COC(=O)CCS)(COC(=O)CCS)COC(=O)CCS]

In some embodiments, the multi-functional thiol crosslinking compound will comprise from about 1% to about 90 mol. %, preferably from about 20 mol. % to about 80 mol. %, and more preferably from about 30 mol. % to about 70 mol. % of said solvent free, injectable, radiopaque composite networks of the present invention. In one or more embodiments, the solvent free, injectable, radiopaque composite network will be from about 20% to about 60%, preferably from about 25% to about 60%, and more preferably from about 30% to about 50% crosslinked after curing.

As will be apparent, the rate of crosslinking at a given temperature will depend upon the molar ratio of allyl groups on said poly(allyl glycidyl ether succinate) copolymer to thiol groups on the di- or multi-functional thiol crosslinking compound. Accordingly, it has ben found that the crosslinking rate can be controlled by varying the concentration of di- or multi-functional thiol crosslinker, varying the degree of polymerization of the PAGES copolymer being used and/or varying the temperature.

In some embodiments, the method may further comprise the step of seeding the radiopaque composite network with mesenchymal stem cells.

In a third aspect, the present invention is directed to a percutaneous polymeric cement for vertebral augmentation comprising the solvent free, injectable, radiopaque composite network described above. In one or more of these embodiments, the percutaneous polymeric cement for vertebral augmentation will further comprise seeding the radiopaque composite network with mesenchymal stem cells.

In yet another aspect, the present invention is directed to a method for vertebral augmentation comprising: preparing a first component comprising a poly(allyl glycidyl ether succinate) copolymer comprising one or more allyl functional groups and a residue of a mono or di or multi-functional initiating alcohol, and a plurality of radiopaque ceramics or metal salt molecules; preparing a second component comprising a multifunctional thiol crosslinker; mixing said first and second component and waiting from about 10 to about 25 min; transferring the mixture into a syringe having a needle sized to be inserted into the damaged area of a vertebra; injecting the mixture into a damaged area of a vertebra and allowing the poly(allyl glycidyl ether succinate) copolymer to crosslink.

In various embodiments, the first component may comprise any of the poly(allyl glycidyl ether succinate) copolymer and radiopaque ceramics or metal salt molecules described above. Similarly, the second component may comprise any of the multifunctional thiol crosslinkers described above. In one or more embodiments the mixture is also seeded with mesenchymal stem cells.

The first and second components are then mixed, starting the crosslinking process. In these embodiments, it is preferable to allow the mixture to crosslink for a time before transferring it to a syringe and/or injecting it into the damaged vertebra of a patient. If the viscosity of the crosslinking mixture is too low, it is difficult if not impossible to keep the PAGES polymer mixture from flowing (or oozing) into undesirable areas and if the viscosity gets too high, the material cannot be injected at all. The waiting time required will depend, at least in part, of the crosslinking rate. As set forth above, it is possible to some extent to control the rate of crosslinking by varying the concentration of di- or multi-functional thiol crosslinker, varying the degree of polymerization of the PAGES copolymer being used and/or varying the temperature. Preferably, however, the crosslinking mixture will be injected before its gel point. In some embodiments, the waiting time will be from about 7 min to about 30 min, in other embodiments, from about 10 min to about 25 min, in other embodiments, from about 10 min to about 20 min, and in other embodiments, from about 15 min to about 20 min. In some embodiments, the waiting time will be from about 15 min.

As set forth above, there are two different percutaneous augmentation strategies in widespread use: vertebroplasty and kyphoplasty. The difference between these two strategies is that kyphoplasty involves drilling into the vertebra and utilizes a balloon to restore the height and shape of the vertebra prior to injection. The present method is suitable to both types of percutaneous augmentation.

In some embodiments, the step of injecting further comprises creating an opening in said damaged vertebra sized to receive the augmentation material. In some embodiments, the step of creating an opening comprises forming a cavity in the vertebra body of said damaged vertebra using a drill or other cutting instrument. In one or more embodiments, the step of injecting and the step of allowing the poly(allyl glycidyl ether succinate) copolymer to crosslink are performed in vivo.

In some embodiments, PAGES and crosslinker were mixed in a glass vial and then transferred to a syringe 15 minutes after the addition of the crosslinker. In these embodiments, the syringe was then connected to an 18 gauge needle which was placed into the drilled cavity in the vertebra body at multiple angles to attempt to get filling into the medullary space of the bone.

In still another aspect, the present invention is directed to a method for treating vertebral compression fractures using the method for vertebral augmentation of described above.

### EXPERIMENTAL

To more fully illustrate and reduce the invention to practice, the PAGES-based composite networks of the present invention were synthesized as set forth above and evaluated

### Effect of Degree of Polymerization on Crosslinking:

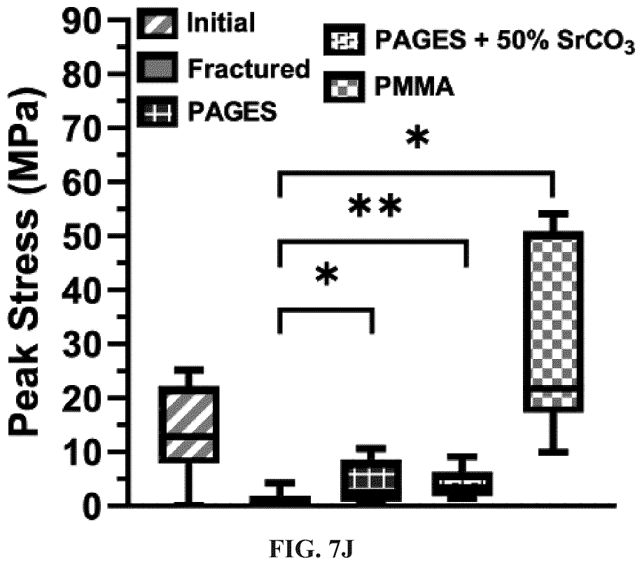

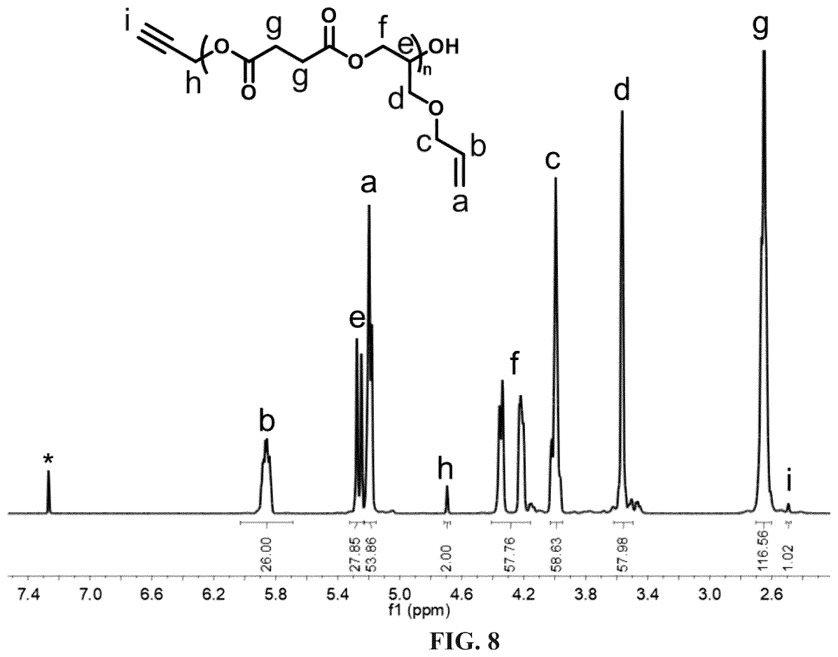

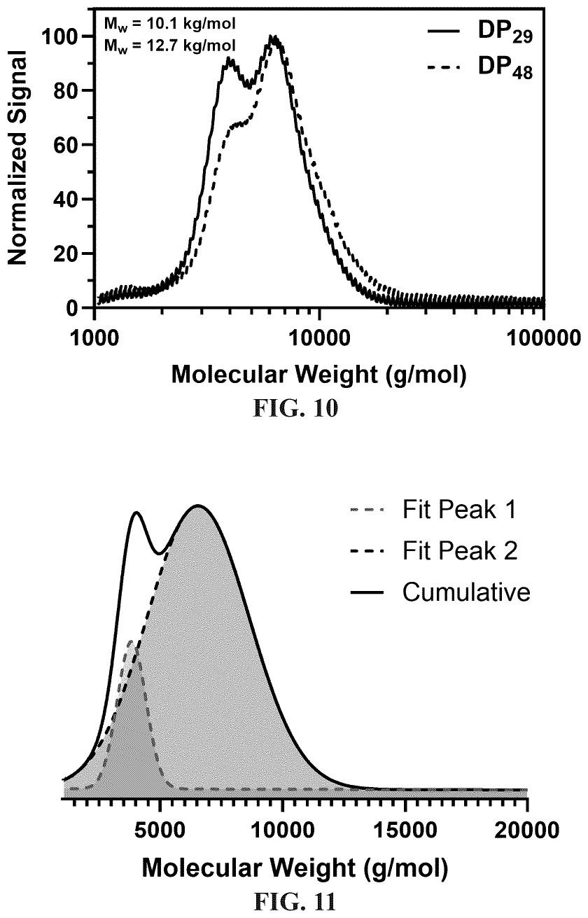

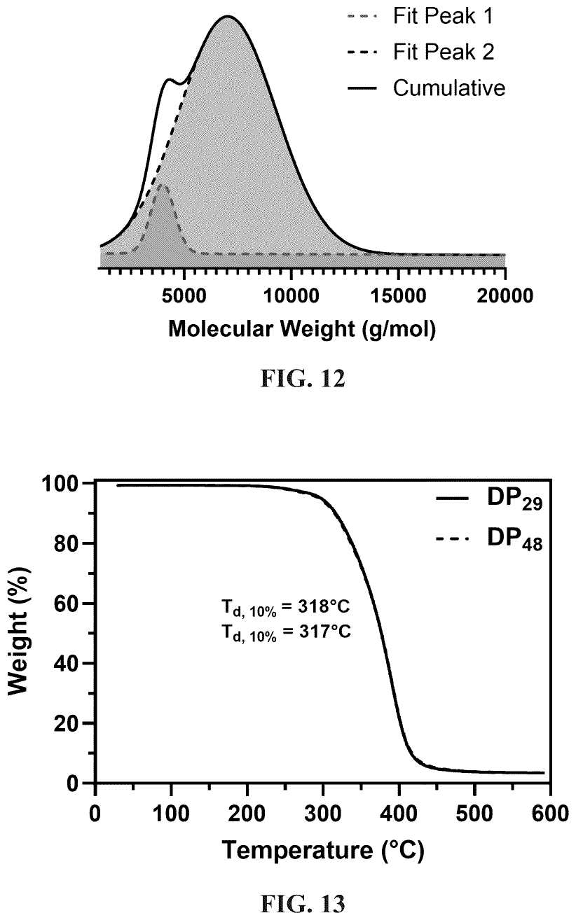

The PAGES was synthesized using ring opening copolymerization of succinic anhydride and allyl glycidyl ether with a propargyl alcohol initiator, as described previously. (See, e.g., M. I. Segal, A. J. Bahnick, N. G. Judge, M. L. Becker, Synthesis and Solvent Free D LP 3D “Printing of Degradable Poly(Allyl Glycidyl Ether Succinate),” Angewandte Chemie (International Ed. in English) (2024). https://doi.org/10.1002/ANIE.202414016; and international application no. PCT/US2024/050531, the disclosures of which are incorporated herein by reference in their entirety). Polymers possessing various degrees of polymerization (DPₙ) were synthesized by altering the ratio of monomer to initiator (Table 1, FIGS. 8-15).

TABLE 1
Reagents used for each polymerization.

| | | Mₙ | Mₙ | M_{w} | | | | |
|---|---|---|---|---|---|---|---|---|
| | | (kDa; | (kDa; | (kDa; | | T_{g} | T_{d} | η |
| [SA]:[AGE]:[I] | Dpₙ | NMR) | SEC)^{a} | SEC)^{b} | Ð^{c} | (° C.)^{d} | (° C.)^{e} | (Pa · s) |
| 30:33:1 | 29 | 6.2 | 8.6 | 10.1 | 1.2 | −34 | 318 | 36 |
| 48:52:1 | 48 | 10.3 | 10.8 | 12.7 | 1.2 | −30 | 317 | 77 |

^{a}Calculated using on M_{w} by MALS and Ð by RI detection using THF SEC.
^{b}Calculated using MALS
^{c}Calculated using RI detection.
^{d}10° C. min⁻¹, second heating cycle.
^{e}10% mass loss, 10° C. min⁻¹
^{f} - 0.1 rad/s to 500 rad/s at 10% strain and 25° C., value reported at 10 rad/s

As the DPₙ increases, the viscosity increases proportionally, as well as the number of potential crosslinking handles present on each monomer repeat unit.

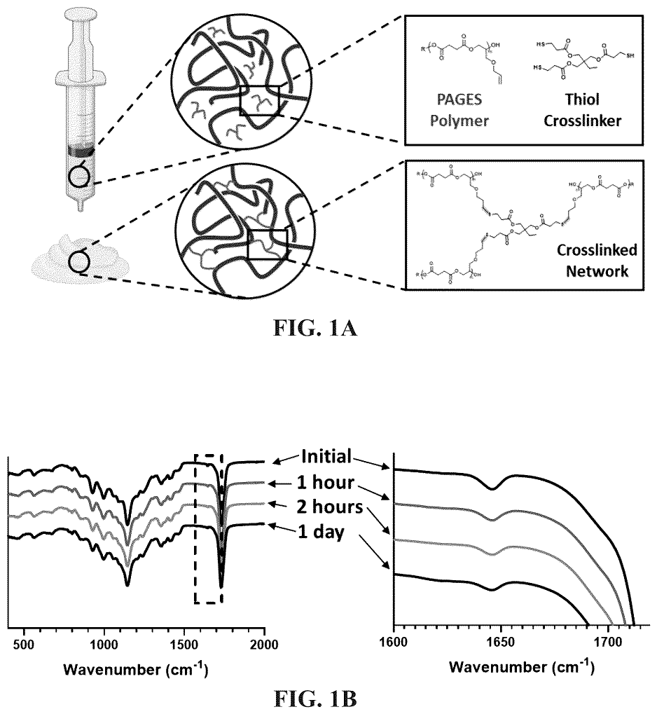

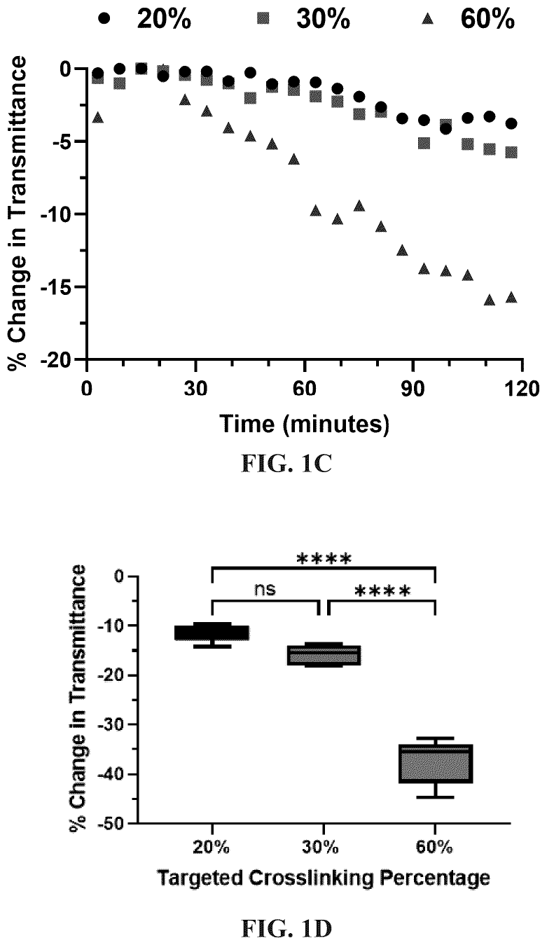

In a series of experiments, PAGES networks and composites were crosslinked under ambient conditions with trimethylolpropane tris(3-mercaptopropionate) via a thiol-ene addition mechanism (FIG. 1A) with 20%, 30%, or 60% of the polymer allyl groups theoretically reacted due to exposure to ambient light during mixing and no added photo initiator. (See, e.g., N. B. Cramer, J. P. Scott, C. N. Bowman, “Photopolymerizations of Thiol-Ene Polymers without Photoinitiators,” Macromolecules 35 (2002) 5361-5365. https://doi.org/10.1021/MA0200672, the disclosure of which is incorporated herein by reference in its entirety). The injectable PAGES polymer and crosslinker mixture forms a covalent network through thiol-ene addition reactions. Specific crosslinking percentages can be targeted by adjusting the stoichiometric ratio of allyl groups in the polymer to thiol groups in the crosslinker. Fourier Transmission Infrared (FTIR) spectroscopy measurements were used to monitor the decrease of the allyl stretching peak (1650 cm⁻¹) (see, e.g., B. C. Smith, “The Infrared Spectroscopy of Alkenes,” Spectroscopy 31 (2016) 28-34, the disclosure of which is incorporated herein by reference in its entirety) in the PAGES spectrum as the crosslinking reaction progresses and allyl groups are consumed (FIG. 1B, Table 2). The theoretical percentage crosslinked and the resulting change in transmittance are reported in Table 2. The average change in normalized allyl stretch intensity at 1650 cm⁻¹ as crosslinking occurs between 15 minutes and 1 day after mixing polymer with crosslinker for samples with varying targeted crosslinking (n=5). The percent change in transmittance values are reported in Table 2 as mean±standard deviation. Furthermore, the normalized decrease in the allyl stretching peak height in the 20%, 30%, and 60% crosslinked samples at 1 day was greater as additional crosslinker was added (FIG. 1C-D). The ratio of the allyl stretching peak height percentage decrease between conditions was proportional to the amount of crosslinker added to the different samples, suggesting that the amount of allyl conversion has a linear relationship with the amount of crosslinker added over the range tested.

TABLE 2

| Theoretical Percentage | Change in |
|---|---|
| crosslinked (%) | Transmittance (%) |
| 20 | −11.5 ± 1.8 |
| 30 | −15.9 ± 2.1 |
| 60 | −37.4 ± 4.6 |

Further, the average gelation time for resins made using polymers of varying DPₙ, varying targeted crosslinking amounts, and varying temperature (n=3) are shown in Table 3, below. Time to gelation values in Table 3 are reported as mean±standard deviation.

TABLE 3

| | Theoretical Percentage | Temperature | Gelation |
|---|---|---|---|
| DPₙ | Crosslinked (%) | (° C.) | Time (min) |
| 48 | 20 | 25 | 161 ± 24 |
| 48 | 30 | 25 | 79 ± 3 |
| 48 | 60 | 25 | 39 ± 6 |
| 48 | 20 | 37 | 53 ± 9 |
| 48 | 30 | 37 | 44 ± 7 |
| 48 | 60 | 37 | 26 ± 1 |
| 29 | 20 | 25 | 49 ± 7 |
| 29 | 30 | 25 | 31 ± 3 |
| 29 | 60 | 25 | 25 ± 3 |
| 29 | 20 | 37 | 33 ± 1 |
| 29 | 30 | 37 | 23 ± 3 |
| 29 | 60 | 37 | 17 ± 3 |

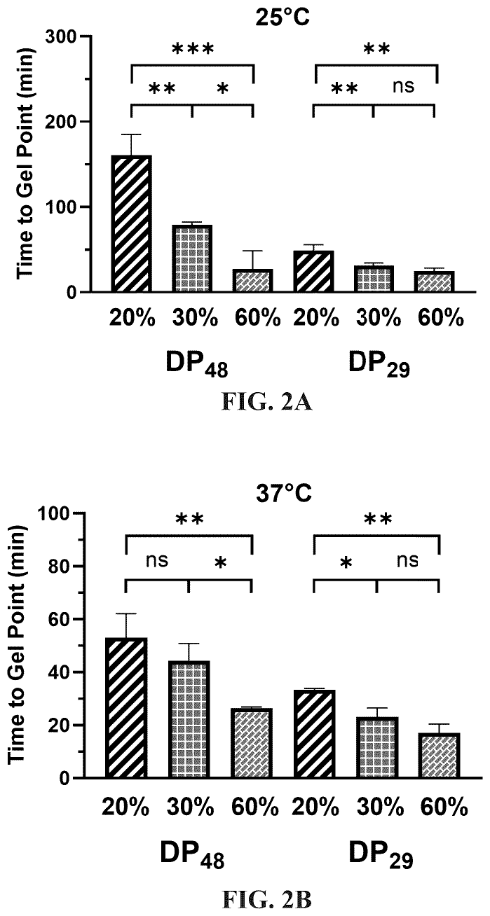

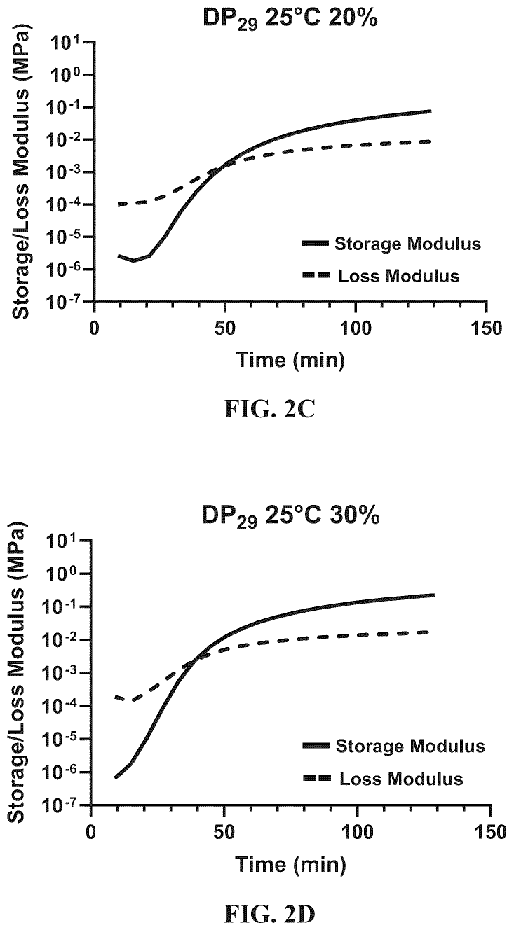

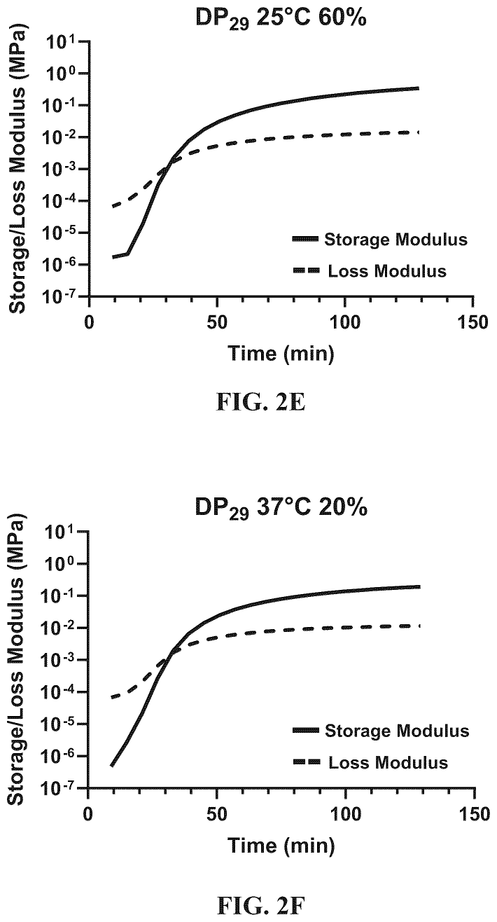

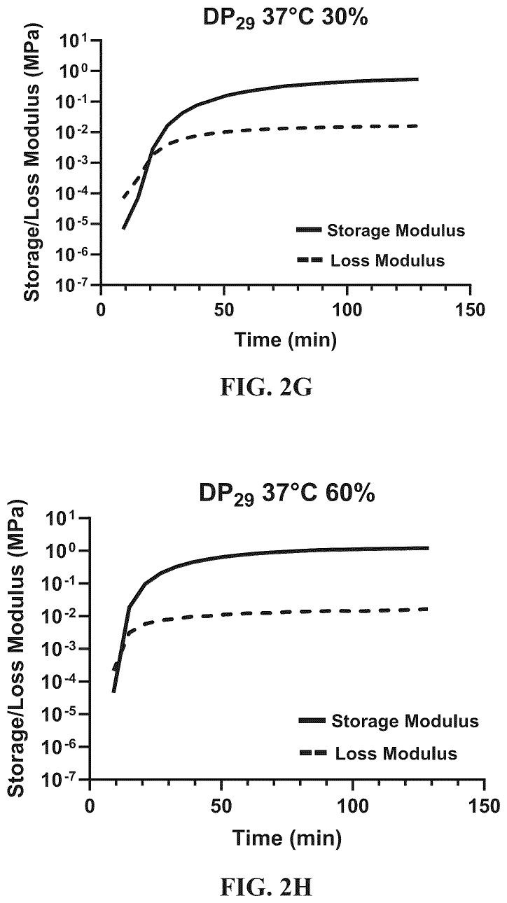

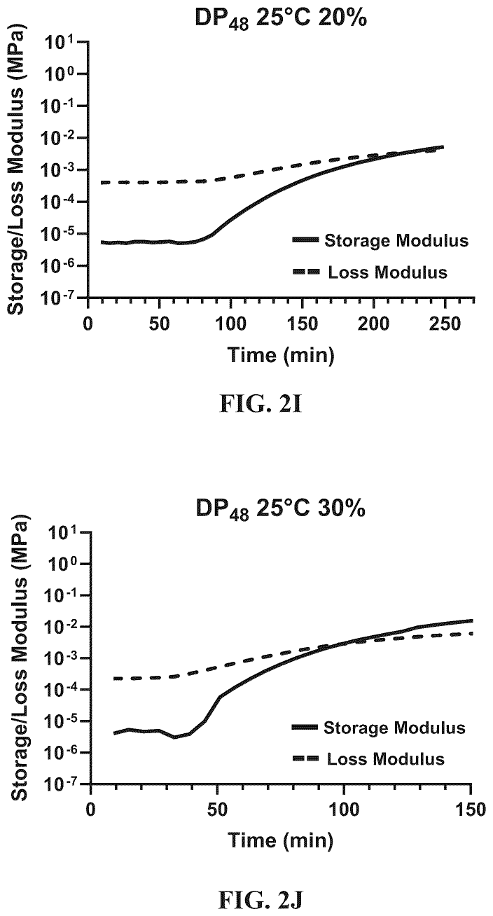

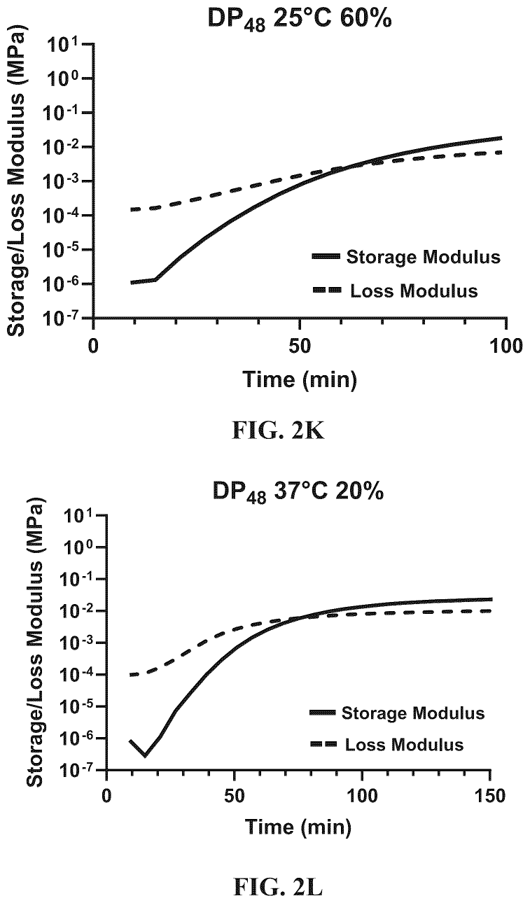

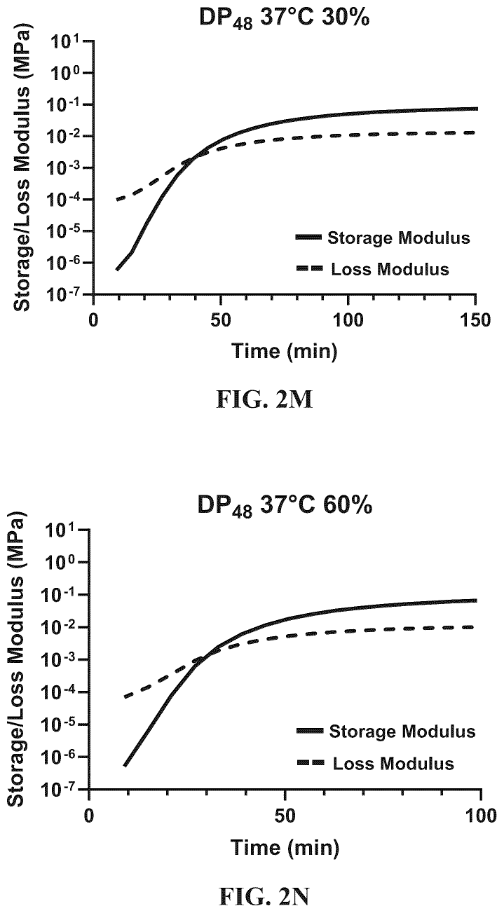

The time to gelation of different PAGES DPₙs and crosslinker percentages were determined using rheological analysis. Time to gel is known to be longer for lower DPₙ related to increased requirements for interchain crosslinking, and larger DPₙ, related to increased viscosity and thus decreased diffusion. (See, e.g., A. P. Dhand, M. D. Davidson, H. M. Zlotnick, T. J. Kolibaba, J. P. Killgore, J. A. Burdick, “Additive manufacturing of highly entangled polymer networks,” Science 385 (2024) 566-572. https://doi.org/10.1126/science.adn6925, the disclosure of which is incorporated herein by reference in its entirety). Based on this, further experiments were performed with DP₂₉ or DP₄₈ PAGES polymers. The time to gel was significantly lower with increasing crosslinking and temperature for both polymers tested (FIGS. 2A-N). The average time to gel of the DP₂₉ with 60% crosslinking was 25±3 minutes at 25° C. and 17±3 minutes at 37° C. compared to 49±7 minutes at 25° C. and 33±1 at 37° C. for DP₄₈ PAGES polymer networks.

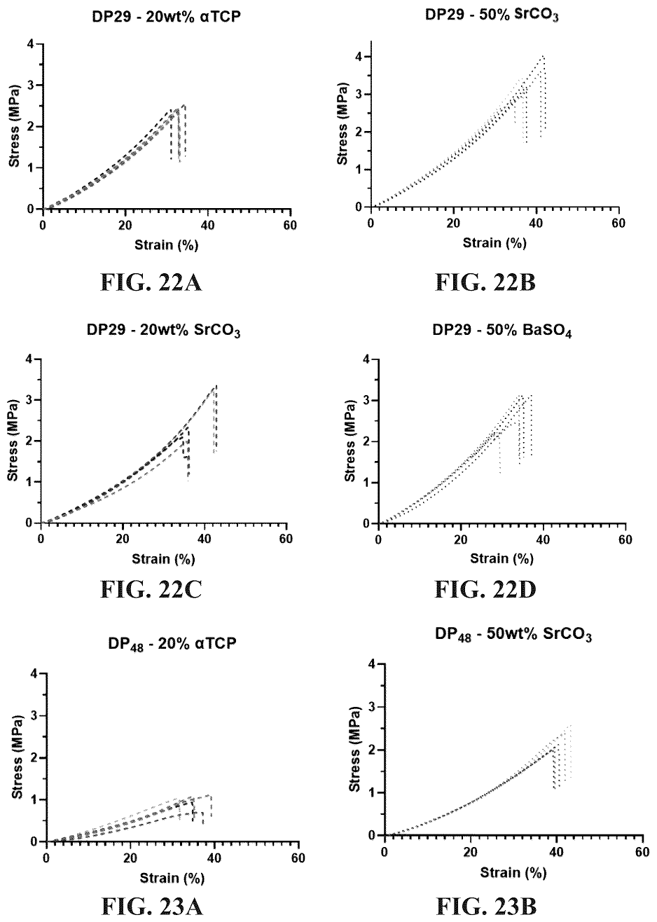

FIGS. 2A-N reflects the results of rheological investigation of gelation time for crosslinking PAGES samples. FIGS. 2A-B show the time to gelation point for PAGES DP₂₉ and DP₄₈ samples with different amounts of targeted crosslinking (20%, 30%, and 60% of allyl groups reacted) at room temperature (25° C.) (n=3) (FIG. 2A) and physiological temperature (37° C.) (n=3) (FIG. 2B). FIGS. 2C-N are graphs showing median sample storage modulus and loss modulus at 10 rad/s as crosslinking occurs over time for DP₂₉ (FIGS. 2C-H) and DP₄₈ (FIGS. 2 I-N) PAGES samples with varying amounts of targeted crosslinking (20% (FIGS. 2C, F, I, L), 30% (FIGS. 22D, G, J, M, and 60% (FIGS. 2E, H, K, N) of allyl groups reacted) at 25° C. (FIGS. 2 C-E, I-K) and 37° C. (FIGS. 2F-H, L-N). The bar charts show mean with error bars representing standard deviation and the degree of statistical significance was determined using Tukey's multiple comparisons test with *p<0.05, **p<0.01, ***p<0.001, ****p<0.0001.

### Degree of Polymerization and Crosslinking Impacts Mechanical Properties

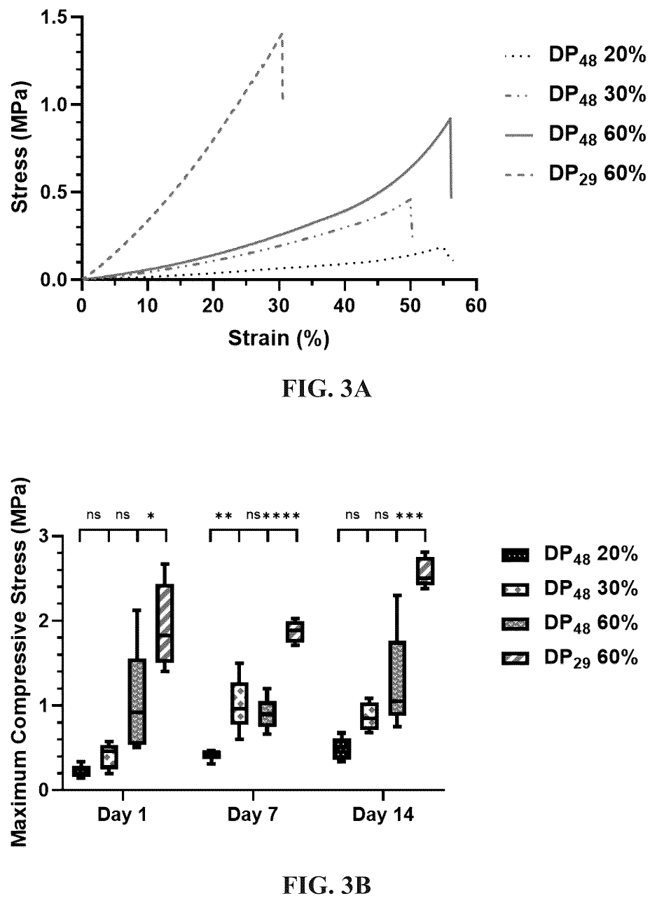

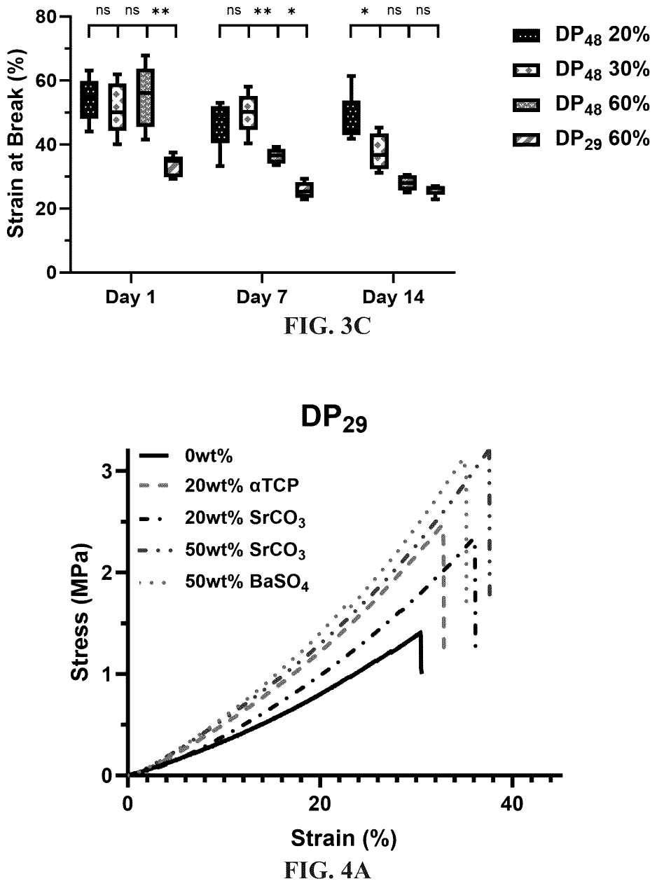

Crosslinked samples were fabricated to determine the compressive properties of the networks. (See, FIGS. 3A-C). FIGS. 3A-C show the compressive strength of PAGES DP₂₉ and DP₄₈ samples with different amounts of targeted crosslinking (20%, 30%, and 60% of allyl groups reacted) allowed to cure under ambient conditions for varying amounts of time (1, 7, and 14 days). FIG. 3A) Median compressive stress-strain curves produced after samples were allowed to cure under ambient conditions for 1 day. FIG. 3B) shows the maximum compressive stress at break for samples allowed to cure under ambient conditions for varying amounts of time (n=5). Similarly, FIG. 3C shows the strain at break for samples allowed to cure under ambient conditions for varying amounts of time (n=5). The box plots in FIGS. 3B-C show median, first quartile, third quartile, maximum, and minimum. The degree of statistical significance determined using Tukey's multiple comparisons test with *p<0.05, **p<0.01, ***p<0.001, ****p<0.0001.

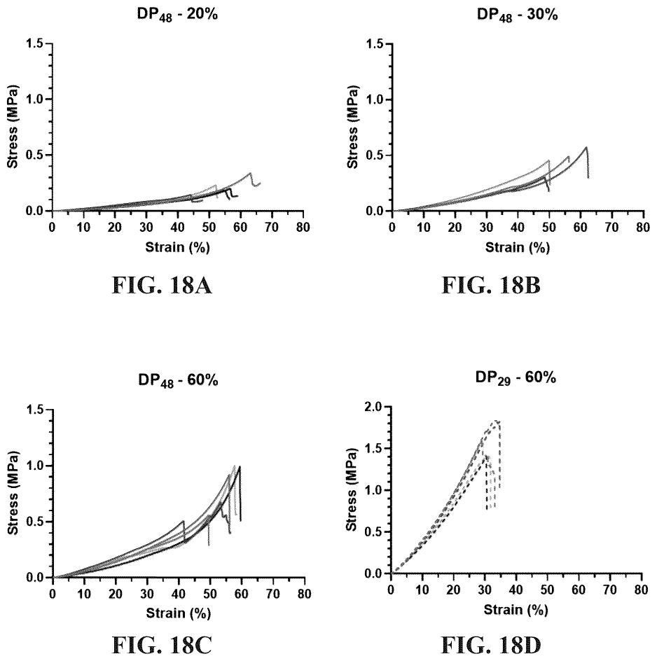

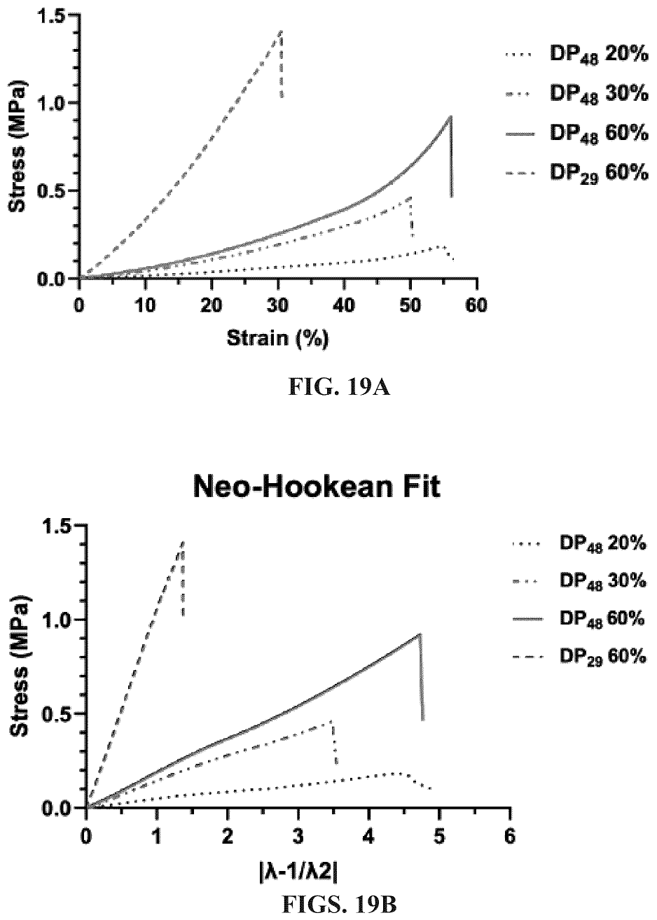

Samples were allowed to cure under ambient conditions for varying amounts of time and were then compressed until failure (FIG. 3A, FIG. 18). We have also applied ideas from rubber elasticity to relate stress to the shear modulus (G) of the samples through a quadratic function of the compression ratio via a Neo-Hookean fitting of the stress-strain curves (FIG. 19 & Table 4). Moduli for PAGES samples with different targeted crosslinking percentages after curing for 1 day under ambient conditions are shown in Table 4. Values for compressive modulus are displayed as mean±95% with a Confidence Interval of n=6 for 60% crosslinked PAGES, n=5 for 20% and 30% crosslinked PAGES. The values for shear modulus reported in Table 4 were calculated from the representative curves with a Neo-Hookean fit (n=1).

TABLE 4

| | Compressive Modulus | Shear Modulus |
|---|---|---|
| Condition | 5-7% Strain (MPa) | (MPa) |
| DP₄₈ 20% | 0.15 ± 0.06 | 0.04 |
| DP₄₈ 30% | 0.39 ± 0.06 | 0.13 |
| DP₄₈ 60% | 0.64 ± 0.10 | 0.19 |
| DP₂₉ 60% | 4.21 ± 0.36 | 1.04 |

After one week, 30% and 60% crosslinked DP₄₈ PAGES samples were significantly stronger than 20% crosslinked DP₄₈ PAGES samples (1±0.3 MPa for 30%, 1.1±0.6 MPa for 60%, 0.4±0.06 MPa for 20%) (FIG. 3B). 60% crosslinked DP₄₈ PAGES samples exhibited stiffening over time with a strain at break of 55±8% after 1 day and a strain at break of 28±2% after 14 days (FIG. 3C). Furthermore, DP₂₉ PAGES samples were found to be significantly less flexible (26±3% compared to 35±3%) and stronger (2.0±0.2 MPa compared to 1.1±0.6 MPa) than DP₄₈ at 60% crosslinking after 7 days (FIG. 3B-C, Table 5).

The maximum compressive stress and compressive strain at break for PAGES samples with different targeted crosslinking percentages that have been allowed to cure under ambient conditions for different amounts of time are reported in Table 5. As can be seen, the PMMA samples express compressive modulus initially (0-1% strain) and stress and strain at the first inflection point in lieu of strain at break and ultimate compressive stress. The PMMA sample compresses, but does not break. The values reported in Table 5 are displayed as mean±95% Confidence Interval (n=6 for 6000 crosslinked PAGES and PMMA, n=5 for 2000 and 3000 crosslinked PAGES except PAGES DP₄₈ 300% Day 14 where n=4).

TABLE 5

| | Maximum | | Compressive |
|---|---|---|---|
| | Compressive | Strain at | Modulus 5-7% |
| Condition | Stress (kPa) | Break (%) | Strain (MPa) |
| DP₄₈ 20% Day 1 | 219 ± 59 | 54.1 ± 5.5 | 0.15 ± 0.06 |
| DP₄₈ 20% Day 7 | 422 ± 51 | 46.8 ± 6.1 | 0.49 ± 0.09 |
| DP₄₈ 20% Day 14 | 490 ± 105 | 47.5 ± 6.2 | 0.62 ± 0.07 |
| DP₄₈ 30% Day 1 | 404 ± 121 | 51.4 ± 6.5 | 0.39 ± 0.06 |
| DP₄₈ 30% Day 7 | 1010 ± 252 | 50.0 ± 5.0 | 0.99 ± 0.09 |
| DP₄₈ 30% Day 14 | 866 ± 144 | 37.5 ± 5.0 | 0.16 ± 0.29 |
| DP₄₈ 60% Day 1 | 1021 ± 512 | 54.9 ± 6.7 | 0.64 ± 0.10 |
| DP₄₈ 60% Day 7 | 1137 ± 538 | 35.1 ± 2.9 | 2.34 ± 0.59 |
| DP₄₈ 60% Day 14 | 1427 ± 535 | 28.4 ± 1.8 | 3.98 ± 1.10 |
| DP₂₉ 60% Day 1 | 1852 ± 439 | 33.0 ± 2.8 | 4.21 ± 0.36 |
| DP₂₉ 60% Day 7 | 1976 ± 249 | 26.3 ± 2.4 | 6.31 ± 0.46 |
| DP₂₉ 60% Day 14 | 2550 ± 146 | 25.7 ± 1.3 | 8.23 ± 0.34 |
| DP₂₉ 60% Day 28 | 2378 ± 410 | 21.7 ± 2.4 | 9.91 ± 0.18 |
| PMMA Day 7 | 12836 ± 3240 | 1.7 ± 0.4 | 860.70 ± 160.40 |
| PMMA Day 14 | 10623 ± 4310 | 2.2 ± 1.0 | 575.60 ± 137.10 |

Since the ultimate compressive stress of human vertebra is reported to be between 1 and 6 MPa, additional testing was performed with the 600% crosslinked PAGES samples. (See, e.g., C. Ohman-Magi, 0. Holub, D. Wu, R. M. Hall, C. Persson, “Density and mechanical properties of vertebral trabecular bone—A review,” JOR Spine 4 (2021) e1176. https://doi.org/10.1002/JSP2.1176, the disclosure of which is incorporated herein by reference in its entirety).

Addition of Ceramics Increases Mechanical Strength without Impacting Gel Time

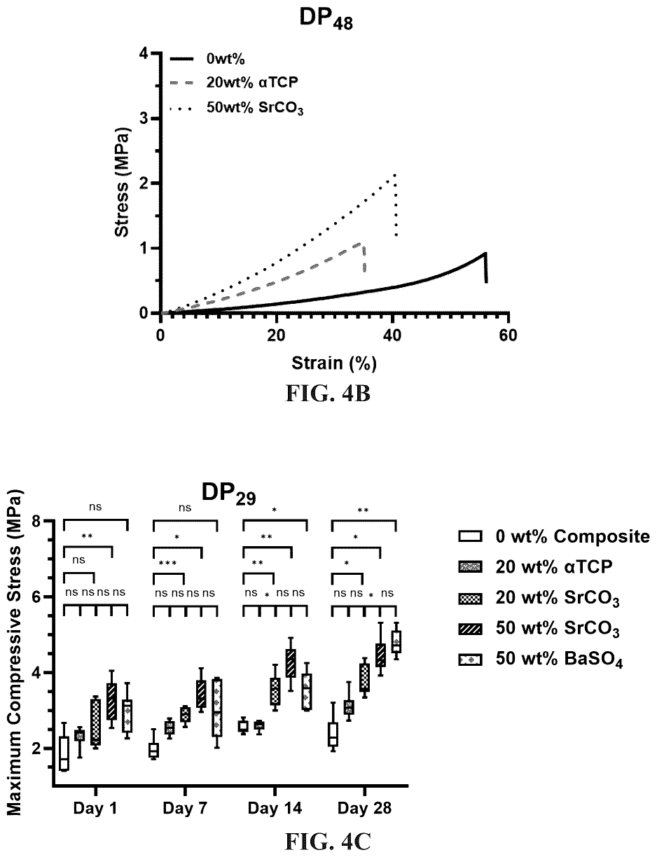

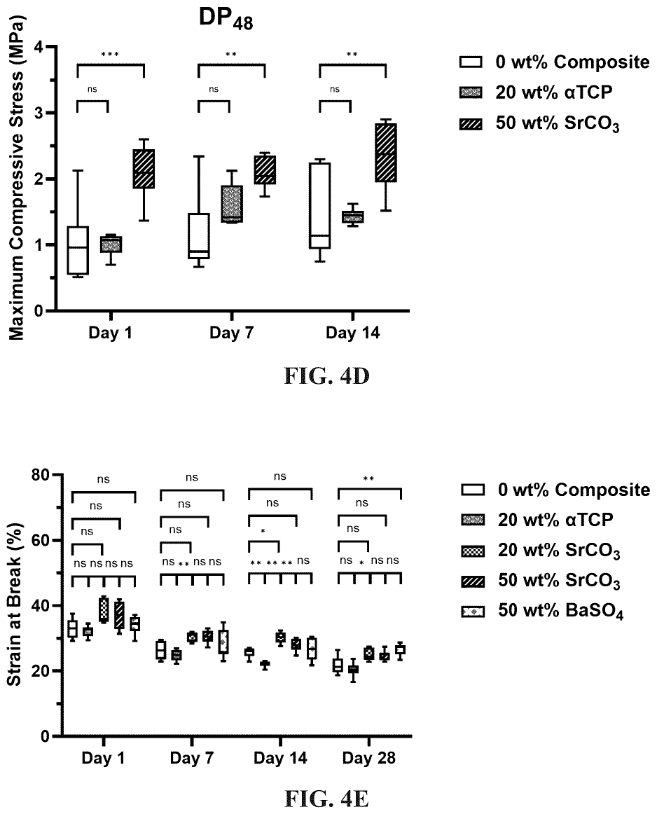

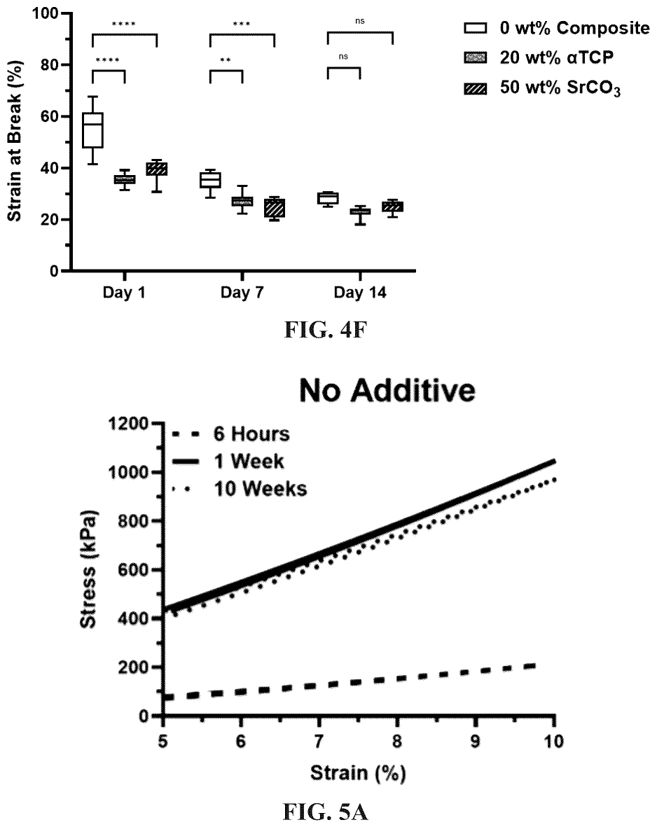

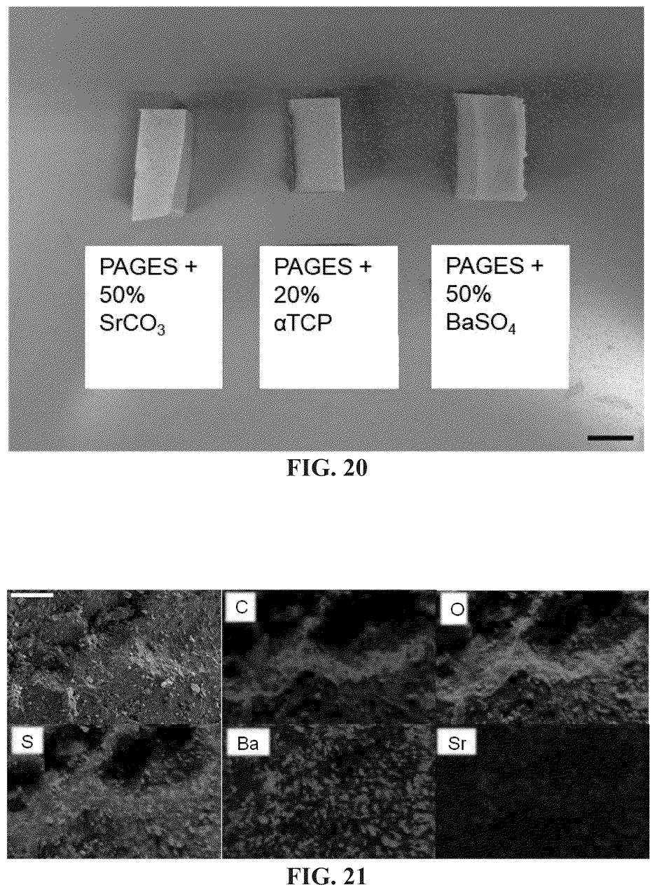

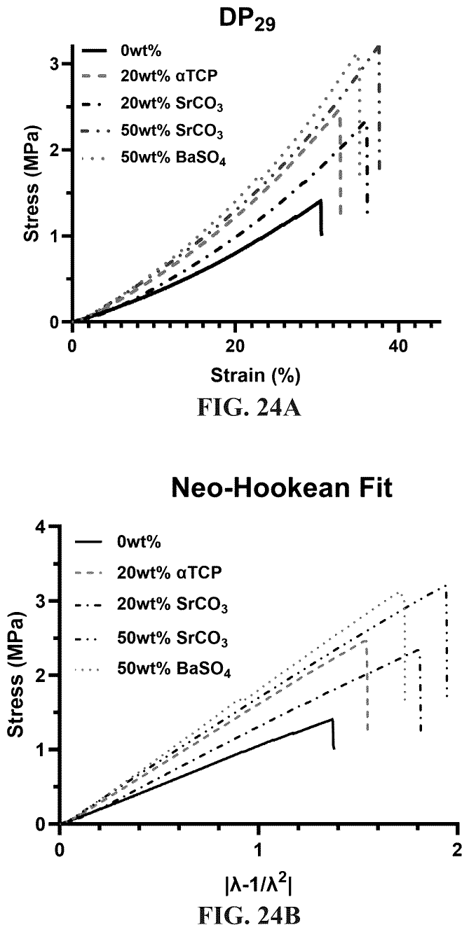

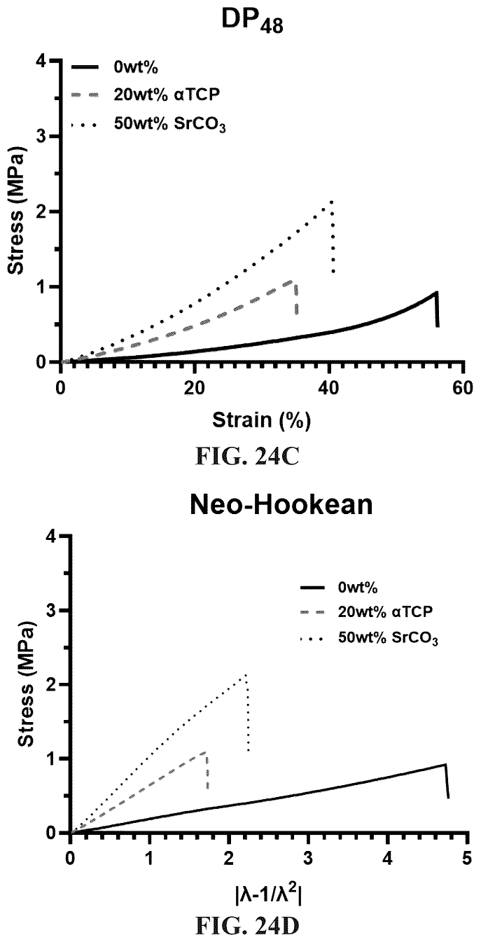

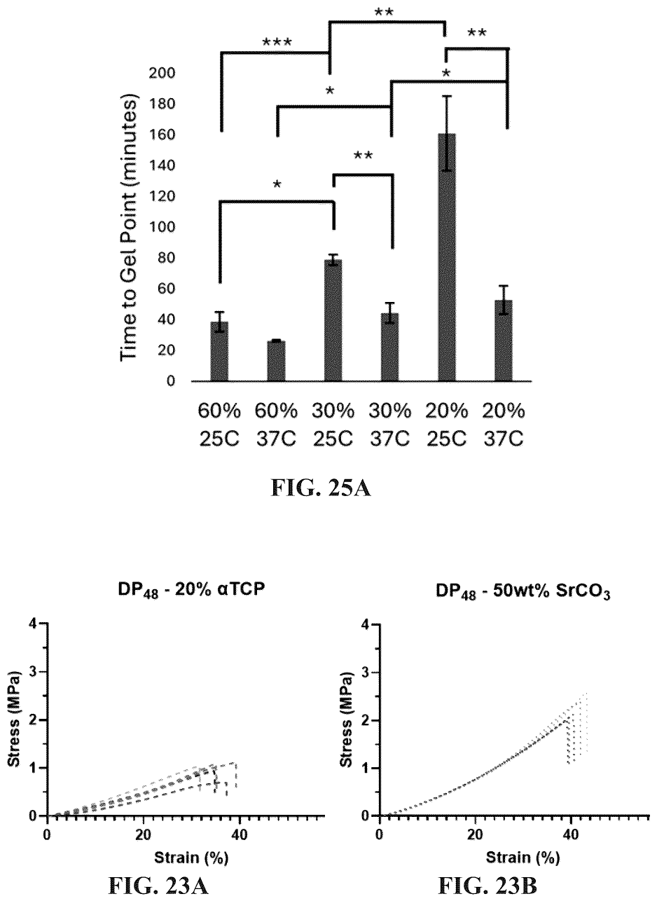

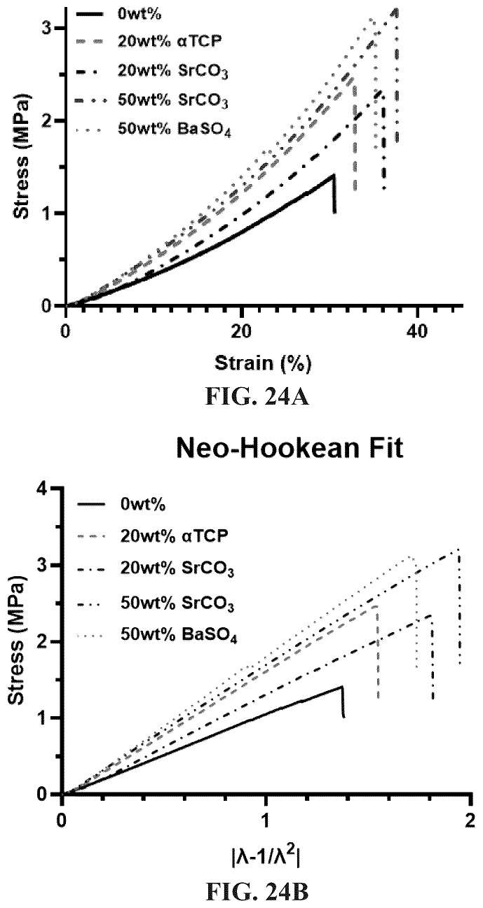

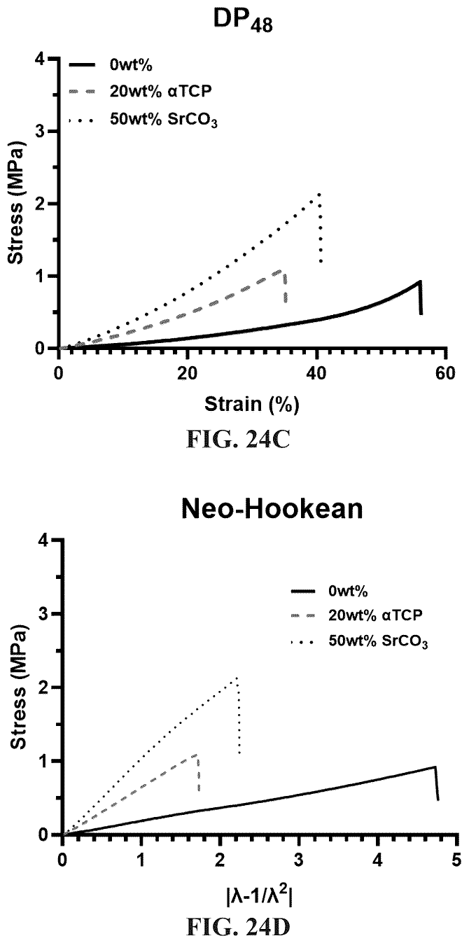

Ceramic nanoparticles (BaSO₄, SrCO₃, or ax-TCP) were added to DP₂₉ PAGES polymer prior to crosslinking to increase the mechanical strength of the resulting composites. BaSO₄ and SrCO₃ were each found to have better qualitative dispersal through the resulting composite polymer than αTCP (FIG. 20) and allowed for crosslinking at higher weight fractions of composites. Microscopic dispersal of SrCO₃ in PAGES samples was further confirmed with scanning electron microscopy (SEM) and energy dispersive X-ray spectroscopy (EDS) (FIG. 21). BaSO₄ and SrCO₃ composite samples demonstrated increased strength compared to both α-TCP and additive free samples at multiple time points for both DP₂₉ and DP₄₈ polymers (FIGS. 4A-F, FIG. 22-24).

FIGS. 4A-F show results from the addition of ceramics to PAGES to increase compressive strength of polymer blocks. In these experiments, the compressive strength of PAGES DP₂₉ and DP₄₈ samples at 60% crosslinking with varying ceramic additives (alpha tricalcium phosphate (αTCP), strontium carbonate (SrCO₃), and barium sulfate (BaSO₄)). The samples were allowed to cure under ambient conditions for varying amounts of time (1, 7, 14 and 28 days) and their compressive strength measured. FIGS. 4A-B are graphs showing the median compressive stress-strain curves produced after PAGES DP₂₉ (FIG. 4A) and DP₄₈ (FIG. 4B) samples were allowed to cure under ambient conditions for 1 day. FIGS. 4C-D are graphs showing the maximum compressive stress at break for PAGES DP₂₉ (FIG. 4C) and DP₄₈ (FIG. 4D) samples allowed to cure under ambient conditions for varying amounts of time (n=5). Finally, FIGS. 4E-F are graphs showing strain at break for PAGES DP₂₉ (FIG. 4E) and DP₄₈ (FIG. 4F) samples allowed to cure for varying amounts of time (n=5). The box plots in FIGS. 4C-F show median, first quartile, third quartile, maximum, and minimum. The degree of statistical significance was determined using Tukey's multiple comparisons test with *p<0.05, **p<0.01, ***p<0.001, ****p<0.0001.

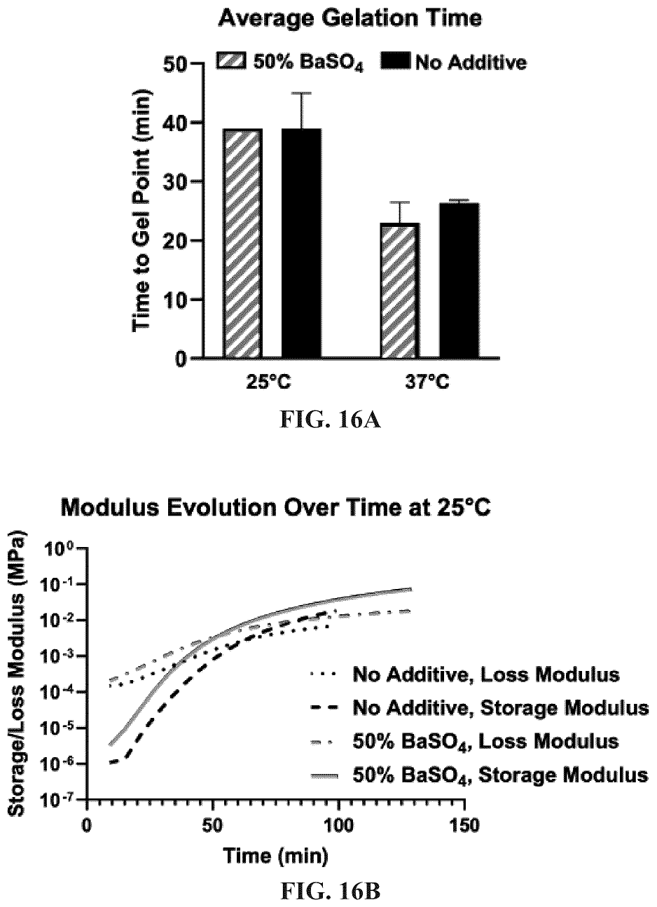

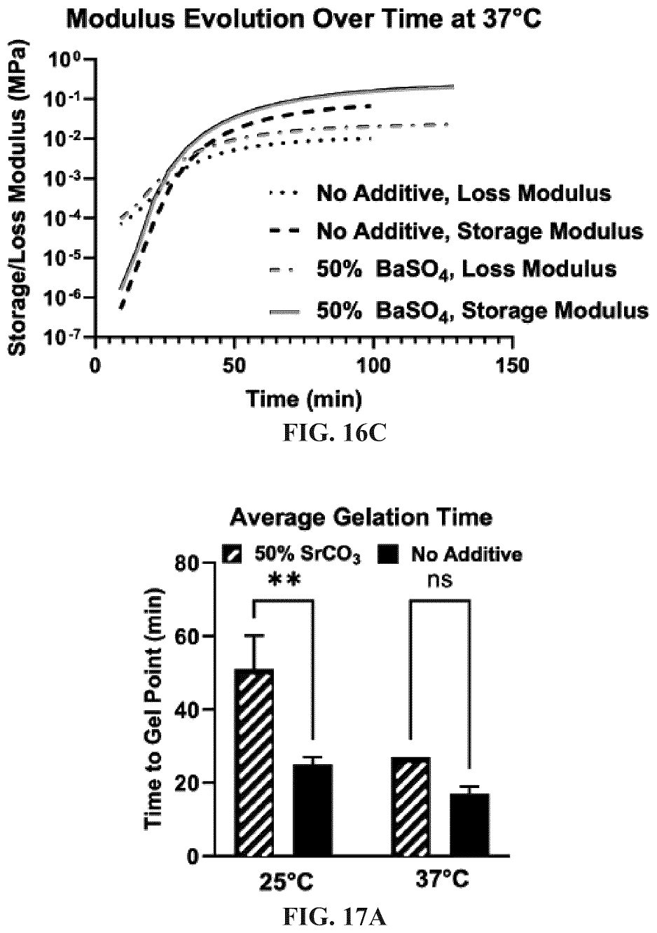

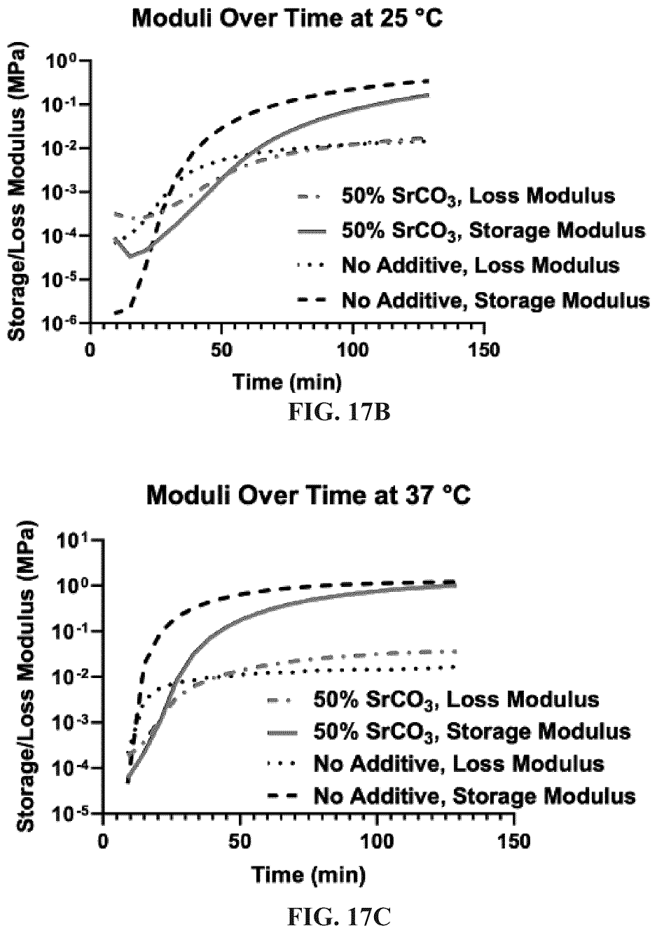

As can be seen, the PAGES samples without additives had a maximum stress of 2.0±0.3 MPa, compared to 2.5±0.2 MPa for 20% αTCP, 3.4±0.4 MPa for 50% SrCO₃, and 3.0±0.7 MPa for 50% BaSO₄ after 7 days of ambient curing (Tables 6-8). There was some mass fraction dependency of the SrCO₃ composite strength with the 50% SrCO₃ samples demonstrating significantly higher ultimate stress than the 20% SrCO₃ samples after 14 days of ambient curing (4.3±0.5 MPa versus 3.5±0.4 MPa). Rheological analysis was used to confirm that the addition of ceramics did not impact the time to gelation (FIGS. 16A-C, 17A-C).

The compressive and sheer moduli for PAGES samples with varying amounts of additives at 60% theoretical crosslinking after curing for 1 day under ambient conditions are shown in Table 6. The values for compressive modulus shown in Table 6 are displayed as mean±95% Confidence Interval (n=6). The values for shear modulus were calculated from the representative curves with a Neo-Hookean fit (n=1).

TABLE 6

| | Compressive Modulus | Shear Modulus |
|---|---|---|
| Condition | 5-7% Strain (MPa) | (MPa) |
| DP₂₉ 0% | 4.21 ± 0.36 | 1.04 |
| DP₂₉ 20% αTCP | 5.35 ± 0.32 | 1.63 |
| DP₂₉ 20% SrCO₃ | 4.48 ± 0.27 | 1.33 |
| DP₂₉ 50% SrCO₃ | 6.38 ± 0.20 | 1.68 |
| DP₂₉ 50% BaSO₄ | 6.15 ± 0.43 | 1.85 |
| DP₄₈ 0% | 0.64 ± 0.10 | 0.19 |
| DP₄₈ 20% αTCP | 2.11 ± 0.50 | 0.65 |
| DP₄₈ 50% SrCO₃ | 3.56 ± 0.23 | 0.98 |

Table 7 reports the compressive measurements (n=6) on composite PAGES polymer blocks having one of BaSO4, SrCO₃, and αTCP as an additive, a DPₙ of 29 or 48, an additive percentage of 20% or 50, and a 602 theoretical crosslinking as these samples were allowed to cure for varying amounts of time under ambient conditions. The materials were tested on day 1 and at weekly intervals for 28 days. The compressive modulus calculated between 5 to 700 strain. Values in Table 7 are reported as mean±9500 Confidence Interval.

TABLE 7

| | Maximum | | Compressive |
|---|---|---|---|
| | Compressive | Strain at | Modulus 5-7% |
| Condition | Strength (kPa) | Break (%) | strain (MPa) |
| DP₂₉ 50% BaSO₄ D 1 | 2972 ± 425 | 34.1 ± 2.3 | 6.15 ± 0.43 |
| DP₂₉ 50% BaSO₄ D 7 | 2997 ± 597 | 27.9 ± 3.6 | 9.02 ± 0.45 |
| DP₂₉ 50% BaSO₄ D 14 | 3550 ± 400 | 26.6 ± 2.9 | 11.24 ± 0.74 |
| DP₂₉ 50% BaSO₄ D 28 | 4786 ± 284 | 26.7 ± 1.5 | 14.33 ± 1.81 |
| DP₂₉ 50% SrCO₃ D 1 | 3273 ± 440 | 37.0 ± 3.3 | 6.38 ± 0.20 |
| DP₂₉ 50% SrCO₃ D 7 | 3413 ± 346 | 30.5 ± 1.6 | 9.31 ± 0.46 |
| DP₂₉ 50% SrCO₃ D 14 | 4274 ± 391 | 27.9 ± 1.6 | 13.08 ± 0.52 |
| DP₂₉ 50% SrCO₃ D 28 | 4447 ± 379 | 24.3 ± 1.3 | 16.23 ± 0.62 |
| DP₂₉ 20% SrCO₃ D 1 | 2529 ± 500 | 37.8 ± 3.0 | 4.48 ± 0.27 |
| DP₂₉ 20% SrCO₃ D 7 | 2889 ± 169 | 30.6 ± 1.2 | 7.47 ± 0.48 |
| DP₂₉ 20% SrCO₃ D 14 | 3548 ± 345 | 30.2 ± 1.5 | 9.41 ± 0.78 |
| DP₂₉ 20% SrCO₃ D 28 | 3764 ± 337 | 24.9 ± 1.5 | 12.96 ± 0.56 |
| DP₂₉ 20% αTCP D 1 | 2323 ± 230 | 32.3 ± 1.4 | 5.35 ± 0.32 |
| DP₂₉ 20% αTCP D 7 | 2534 ± 157 | 24.8 ± 1.4 | 8.88 ± 0.21 |
| DP₂₉ 20% αTCP D 14 | 2602 ± 1092 | 22.1 ± 0.7 | 10.81 ± 0.32 |
| DP₂₉ 20% αTCP D 28 | 3113 ± 274 | 20.4 ± 1.8 | 14.49 ± 1.44 |

Table 8 reports the compressive measurements (n=6) on composite PAGES polymer blocks having SrCO₃ of αTCP as an additive, a DPₙ of 48, an additive percentage of 20% or 50%, and a 6000 theoretical crosslinking as these samples were allowed to cure for varying amounts of time under ambient conditions. The materials were tested on day 1 and at weekly intervals for 14 days. The compressive modulus calculated between 5 to 70% strain. Values in Table 8 are reported as mean±9500 Confidence Interval.

TABLE 8

| | Maximum | | Compressive |
|---|---|---|---|
| | Compressive | Strain at | Modulus 5-7% |
| Condition | Strength (kPa) | Break (%) | strain (MPa) |
| DP₄₈ 50% SrCO₃ D 1 | 2093 ± 421 | 39.1 ± 4.3 | 3.56 ± 0.23 |
| DP₄₈ 50% SrCO₃ D 7 | 2087 ± 248 | 25.1 ± 3.8 | 7.52 ± 0.94 |
| DP₄₈ 50% SrCO₃ D 14 | 2345 ± 519 | 25.0 ± 2.4 | 8.38 ± 1.10 |
| DP₄₈ 20% αTCP D 1 | 1008 ± 169 | 35.4 ± 2.5 | 2.11 ± 0.50 |
| DP₄₈ 20% αTCP D 7 | 1576 ± 325 | 27.3 ± 3.5 | 5.74 ± 0.97 |
| DP₄₈ 20% αTCP D 14 | 1438 ± 118 | 23.0 ± 2.5 | 6.34 ± 0.66 |

### Degradation of Cross/Inked PAGES Network and Composite Samples

Crosslinked PAGES networks degrade through hydrolysis of ester bonds within the polymer backbone and the trimethylolpropane tris(3-mercaptopropionate) crosslinker. Two degradation studies were performed by mass and recurring mechanical measurements to determine network degradation rate and changes in mechanical properties over time (Tables 9 and 10). The results of a first degradation study for PAGES composites having no additive, BaSO₄, and SrCO₃ are reported in Table 9, which shows the mass (n=6) as a percentage of initial value over time in 1×PBS or 1 M HCl solution for all composites. The values in Table 9 are reported as Mean±Standard Deviation.

TABLE 9

| | Mass of | Mass of | Mass of | Mass of | Mass of | Mass of |
|---|---|---|---|---|---|---|
| | No | No | BaSO₄ | BaSO₄ | SrCO₃ | SrCO₃ |
| | Additive | Additive | Composite | Composite | Composite | Composite |
| Time | in PBS | in HCl | in PBS | in HCl | in PBS | in HCl |
| (Weeks) | (%) | (%) | (%) | (%) | (%) | (%) |
| 0.14 | 101.6 ± 0.2 | 101.3 ± 0.2 | 101.3 ± 0.2 | 100.7 ± 0.1 | 101.2 ± 0.2 | 101.6 ± 0.3 |
| 1 | 102.0 ± 0.2 | 101.4 ± 0.2 | 102.5 ± 0.2 | 101.1 ± 0.1 | 101.8 ± 0.1 | 101.5 ± 0.4 |
| 2 | 102.0 ± 0.2 | 101.2 ± 0.2 | 102.7 ± 0.2 | 101.3 ± 0.1 | 101.8 ± 0.1 | 99.93 ± 0.2 |
| 3 | 102.1 ± 0.3 | 100.8 ± 0.3 | 103.0 ± 0.3 | 101.2 ± 0.2 | 101.7 ± 0.3 | 98.11 ± 1.2 |
| 4 | 102.0 ± 0.2 | 99.71 ± 0.7 | 103.1 ± 0.2 | 101.0 ± 0.2 | 101.7 ± 0.1 | 97.52 ± 0.4 |
| 5 | 102.4 ± 0.1 | 99.11 ± 0.8 | 103.7 ± 0.2 | 101.1 ± 0.2 | 102.2 ± 0.1 | 97.59 ± 0.3 |
| 6 | 102.1 ± 0.4 | 96.20 ± 1.6 | 103.5 ± 0.3 | 100.6 ± 0.4 | 101.8 ± 0.2 | 94.77 ± 0.5 |
| 7 | 102.5 ± 0.2 | 93.99 ± 2.1 | 103.6 ± 0.1 | 99.49 ± 0.5 | 102.1 ± 0.2 | 93.95 ± 0.7 |
| 8 | 102.4 ± 0.3 | 90.57 ± 2.2 | 103.9 ± 0.2 | 98.50 ± 0.8 | 102.0 ± 0.2 | 92.12 ± 1.1 |
| 9 | 102.7 ± 0.1 | 84.85 ± 3.2 | 103.7 ± 0.3 | 95.51 ± 1.5 | 102.4 ± 0.2 | 89.64 ± 1.0 |
| 10 | 102.6 ± 0.3 | 80.29 ± 3.2 | 103.8 ± 0.5 | 93.34 ± 2.7 | 102.3 ± 0.6 | 87.70 ± 1.2 |
| 11 | 102.7 ± 0.3 | 75.27 ± 3.0 | 104.0 ± 0.4 | 85.88 ± 1.9 | 102.3 ± 0.3 | 85.05 ± 1.4 |
| 12 | 102.8 ± 0.2 | 70.76 ± 2.7 | 104.1 ± 0.4 | 81.09 ± 0.9 | 102.7 ± 0.3 | 82.66 ± 1.7 |
| 13 | 102.7 ± 0.2 | 66.70 ± 2.6 | 104.2 ± 0.3 | 76.16 ± 0.7 | 102.4 ± 0.3 | 80.20 ± 1.7 |
| 14 | 102.8 ± 0.3 | 62.89 ± 2.8 | 104.2 ± 0.3 | 72.20 ± 0.5 | 102.3 ± 0.2 | 78.14 ± 1.9 |
| 15 | 102.6 ± 0.3 | 59.53 ± 2.7 | 104.6 ± 0.4 | 69.27 ± 0.7 | 102.2 ± 0.3 | 76.43 ± 1.9 |
| 16 | 102.8 ± 0.3 | 55.83 ± 2.7 | 104.5 ± 0.4 | 65.72 ± 0.7 | 102.4 ± 0.3 | 74.50 ± 2.0 |
| 17 | 102.8 ± 0.4 | 50.85 ± 2.7 | 104.4 ± 0.3 | 61.03 ± 0.9 | 102.3 ± 0.2 | 72.05 ± 2.2 |
| 18 | 102.8 ± 0.3 | 47.99 ± 2.8 | 104.5 ± 0.3 | 58.87 ± 1.0 | 102.5 ± 0.3 | 69.97 ± 2.4 |
| 19 | 103.0 ± 0.4 | 44.76 ± 2.9 | 104.6 ± 0.3 | 55.09 ± 1.0 | 102.5 ± 0.3 | 68.36 ± 2.4 |
| 20 | 103.6 ± 0.7 | 42.83 ± 2.7 | 105.3 ± 0.5 | 53.13 ± 1.3 | 103.4 ± 0.2 | 67.73 ± 2.9 |
| 21 | 103.9 ± 0.5 | 38.39 ± 2.7 | 105.1 ± 0.4 | 48.55 ± 1.2 | 103.3 ± 0.2 | 65.01 ± 2.9 |
| 22 | 103.2 ± 0.3 | 35.39 ± 2.7 | 104.9 ± 0.4 | 45.76 ± 1.3 | 102.6 ± 0.3 | 62.36 ± 2.9 |
| 23 | 103.2 ± 0.3 | 32.90 ± 2.7 | 105.0 ± 0.4 | 42.24 ± 1.3 | 102.6 ± 0.2 | 60.63 ± 3.1 |
| 24 | 103.4 ± 0.3 | 29.68 ± 2.7 | 105.1 ± 0.4 | 39.01 ± 1.6 | 102.7 ± 0.2 | 58.35 ± 3.3 |
| 25 | 103.6 ± 0.3 | 27.45 ± 2.8 | 105.3 ± 0.3 | 36.61 ± 1.7 | 102.9 ± 0.3 | 56.76 ± 3.3 |
| 26 | 103.8 ± 0.3 | 25.05 ± 2.7 | 105.5 ± 0.4 | 34.30 ± 1.7 | 102.9 ± 0.4 | 54.38 ± 3.5 |

The results of a second degradation study for PAGES composites having no additive, BaSO4, and SrCO₃ are reported in Table 10, which shows the compressive modulus (n=6) between 5 to 700 strain for all composites (No Additive, BaSO₄, and SrCO₃) over time in a 1×PBS or 1 M HCl solution. The values in Table 10 are reported as Mean±Standard Deviation.

TABLE 10

| | No | No | BaSO₄ | BaSO₄ | SrCO₃ | SrCO₃ |
|---|---|---|---|---|---|---|
| | Additive | Additive | Composite | Composite | Composite | Composite |
| Time | in PBS | in HCl | in PBS | in HCl | in PBS | in HCl |
| (Weeks) | (MPa) | (MPa) | (MPa) | (MPa) | (MPa) | (MPa) |
| 0 | 1.76 ± 0.46 | 2.48 ± 0.45 | 3.27 ± 0.93 | 4.43 ± 1.15 | 3.79 ± 1.13 | 3.79 ± 1.13 |
| 0.14 | 6.10 ± 0.85 | 6.89 ± 0.79 | 7.68 ± 1.12 | 9.15 ± 0.47 | 8.41 ± 0.28 | 9.63 ± 1.71 |
| 1 | 12.55 ± 1.87 | 12.65 ± 2.14 | 12.77 ± 2.65 | 13.39 ± 3.15 | 12.98 ± 3.00 | 11.67 ± 1.91 |
| 2 | 10.89 ± 0.70 | 12.03 ± 0.88 | 12.36 ± 2.86 | 13.58 ± 1.92 | 16.20 ± 1.26 | 16.03 ± 2.72 |
| 3 | 11.83 ± 0.42 | 12.13 ± 0.93 | 11.83 ± 0.91 | 13.32 ± 0.65 | 16.11 ± 3.68 | 17.88 ± 1.18 |
| 4 | 11.82 ± 0.82 | 12.35 ± 0.69 | 12.30 ± 0.44 | 13.92 ± 0.56 | 17.03 ± 1.90 | 16.90 ± 2.95 |
| 6 | 12.40 ± 0.50 | 11.25 ± 1.07 | 11.30 ± 1.80 | 12.27 ± 0.91 | 16.03 ± 2.71 | 18.02 ± 1.72 |
| 8 | 12.17 ± 1.21 | 10.42 ± 0.62 | 11.98 ± 1.02 | 11.13 ± 0.52 | 14.67 ± 2.50 | 17.65 ± 2.13 |
| 10 | 11.77 ± 1.86 | 10.08 ± 0.69 | 12.13 ± 0.92 | 9.77 ± 0.52 | 15.53 ± 2.54 | 18.65 ± 1.27 |
| 12 | 12.23 ± 1.56 | 10.38 ± 0.46 | 12.37 ± 1.32 | 10.15 ± 0.60 | 19.42 ± 1.77 | 15.72 ± 2.81 |
| 14 | 12.83 ± 1.40 | 10.66 ± 0.60 | 12.95 ± 1.30 | 10.17 ± 0.48 | 16.62 ± 2.96 | 18.30 ± 2.27 |
| 18 | 12.52 ± 1.32 | 10.27 ± 0.74 | 12.67 ± 1.31 | 9.93 ± 0.75 | 15.00 ± 2.55 | 19.87 ± 1.02 |
| 22 | 12.17 ± 1.21 | 8.69 ± 1.52 | 12.20 ± 0.97 | 8.27 ± 1.32 | 19.70 ± 2.24 | 15.13 ± 2.33 |
| 26 | 11.53 ± 1.12 | 7.32 ± 1.83 | 12.08 ± 0.99 | 7.38 ± 1.94 | 19.37 ± 2.43 | 14.13 ± 2.37 |

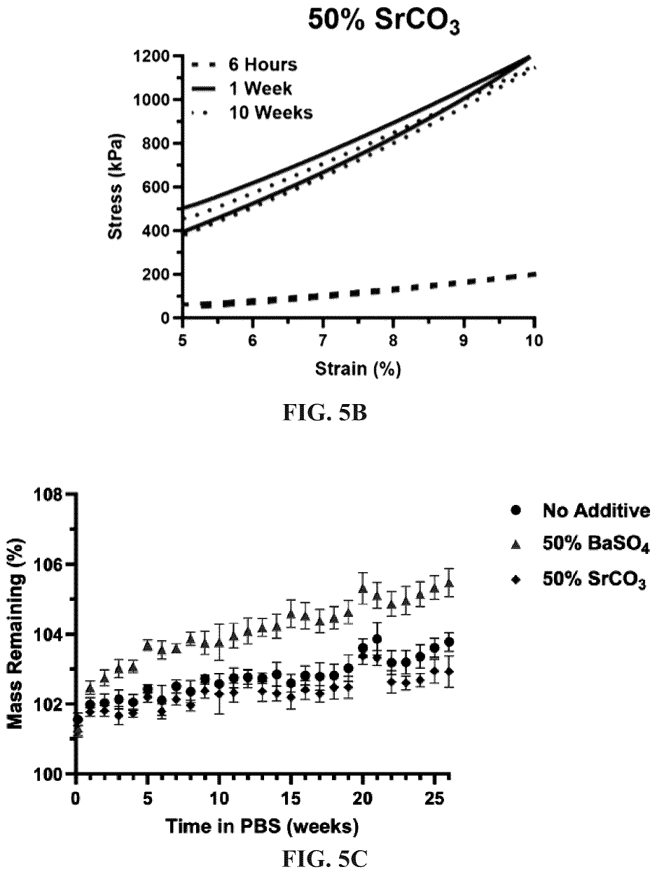

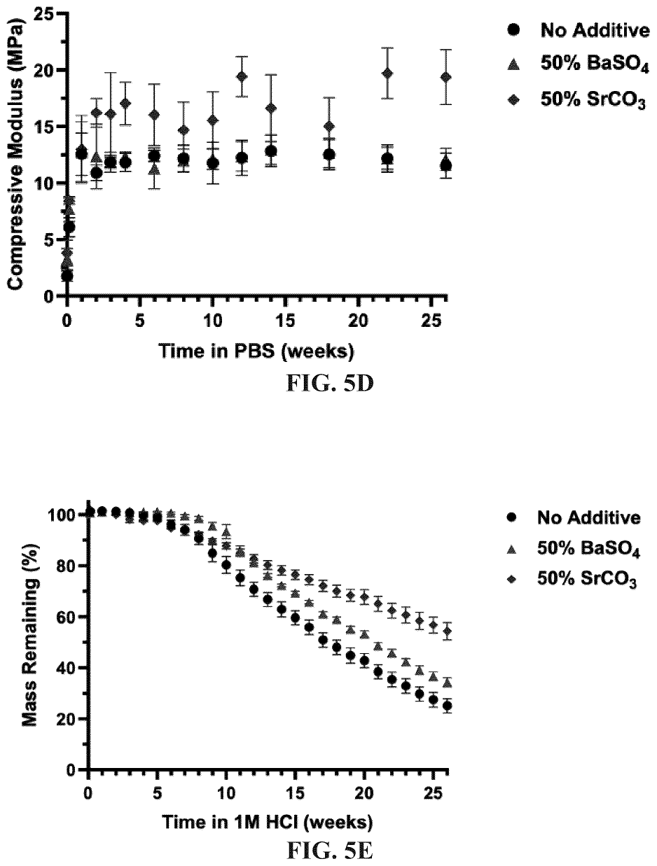

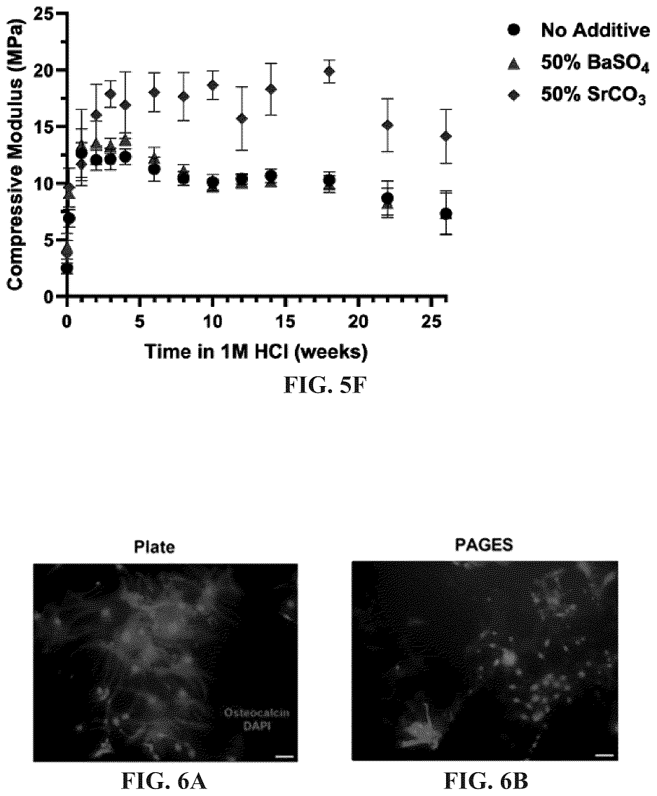

The samples were incubated at 37° C. with agitation for the duration of the study. Degradation in an acidic solution of 1M HCl was chosen to promote accelerated environmental degradation. (See, e.g., M. Bartnikowski, T. R. Dargaville, S. Ivanovski, D. W. Hutmacher, “Degradation mechanisms of polycaprolactone in the context of chemistry, geometry and environment,” Progress in Polymer Science 96 (2019) 1-20. https://doi.org/i0.1016/J. PROGPOLYMVSCI.2019.05.004, the disclosure of which is incorporated herein by reference in its entirety). FIGS. 5A-E are a series of graphs showing the retained mechanical strength exhibited by PAGES DP₂₉, 6000 crosslinked materials while degrading. FIGS. 5A-B are representative compressive loading and unloading curves of PAGES samples without any ceramic additive (FIG. 5A) and with 50% strontium carbonate (SrCO₃) additive (FIG. 5B) after degrading in 1M HCl for varying amounts of time (6 hours, 1 week, 10 weeks). FIG. 5C is a graph showing the mass of PAGES samples without any ceramic additive, with 50% SrCO₃ additive, and with 50% BaSO₄ additive after degrading in PBS for up to 26 weeks (n=6). FIG. 5D is a graph showing the compressive modulus of PAGES samples without any ceramic additive, with 50% SrCO₃ additive, and with 50% BaSO₄ additive after degrading in PBS for up to 26 weeks (n=6). FIG. 5E) is a graph showing the mass of PAGES samples without any ceramic additive, with 50 w % SrCO₃ additive, and with 50% BaSO₄ additive after degrading in 1M HCl for up to 26 weeks (n=6) and FIG. 5F is a graph showing the compressive modulus of PAGES samples without any ceramic additive, with 50% SrCO₃ additive, and with 50% BaSO₄ additive after degrading in 1M HCl for up to 26 weeks (n=6). Plotted data points show mean with error bars representing standard deviation.

Crosslinked samples incubated in phosphate buffered saline (pH 7.4, PBS) demonstrated a maximum 5% swelling without significant mass loss over 26 weeks (FIG. 5C). Samples in 1M HCl begun to demonstrate mass loss at 4 weeks (FIG. 5E). To compare our analysis relevant clinical models on vertebrae fracture, we have calculated the values of the compressive modulus based on a linear fit of data between 5% and 7% strain. (See, e.g., M. M. Panjabi, A. A. I. White, Basic Biomechanics of the Spine, Neurosurgery 7 (1980) 76; and T. Brown, R. J. Hansen, A. J. Yorra, “Some Mechanical Tests on the Lumbosacral Spine with Particular Reference to the Intervertebral Discs: A Preliminary Report,” JBJS 39 (1957) 1135, the disclosures of which are incorporated herein by reference in their entirety). All samples exhibited an increased compressive modulus over the first week likely due to continued crosslinking, as was also observed in the ambient condition samples (FIG. 5D-F). A small decrease in compressive modulus was seen in all 1M HCl samples after 6 weeks, although it then stabilized through 18 weeks before further decreasing in the additive free networks and 50% BaSO₄ composite networks (FIG. 5F). The lack of degradation in the 50% SrCO₃ networks is thought to be related to a buffering effect of the CO₃²⁻ and ultimately HCO₃⁻ arising from the SrCO₃ containing polymer samples that slowed the acid mediated degradation. Mechanical strength of the samples in PBS was maintained throughout the duration of the study (FIG. 5D). These findings demonstrate that PAGES maintains its strength even while degrading, suggesting that it will continue to provide augmentation support while being absorbed. The maintenance of mechanical properties during degradation is a desirable property as it may help to keep the augmented vertebral body from compressing further due to mechanical strength loss.

### Addition of SrCO₃Increases Osteoblast Differentiation

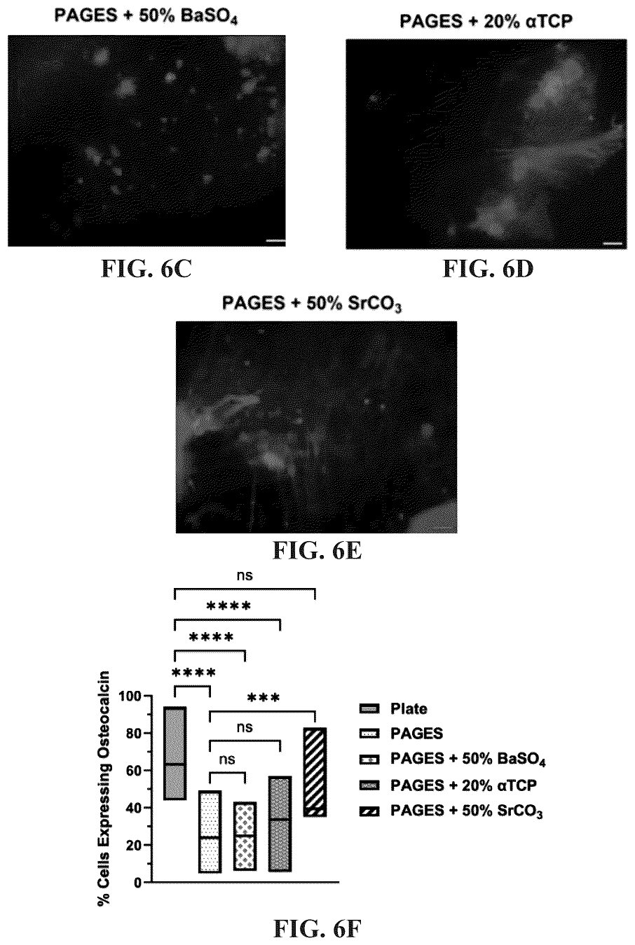

Rat mesenchymal stem cells (MSCs) were cultured on thin-layers of PAGES networks with and without ceramic additives. To assess osteoblast differentiation, cells were stained for osteocalcin after 17 days in culture and the percentage of nuclei that colocalized with osteocalcin was determined. As can be seen, MSCs attached to and proliferated on all PAGES network coated plates demonstrating that PAGES is nontoxic and biocompatible (FIGS. 6A-E). FIGS. 6A-F show the results of an in vitro analysis of DP₂₉ PAGES, 60% crosslinked materials with ceramic additives. FIGS. 6A-E are representative images of rat mesenchymal stem cells (MSCs) after 17 days of culture on either tissue culture plates or a thin layers of PAGES network with or without additives (alpha tricalcium phosphate (αTCP), strontium carbonate (SrCO₃), and barium sulfate (BaSO₄)). All scale bars represent 50 m and the blue color represents cell nuclei stained using DAPI, and red color represents osteocalcin. FIG. 6F is a graph showing quantification of percentage of cells expressing osteocalcin (n=9). The box plots show median, maximum, and minimum and the degree of statistical significance determined using Tukey's multiple comparisons test with *p<0.05, **p<0.01, ***p<0.001, ****p<0.0001.

Adding SrCO₃ to the PAGES samples resulted in significantly increased osteoblast differentiation compared to any other PAGES network (53±8% for PAGES±50% SrCO₃ versus 26±7% for PAGES) and was not significantly different to what was seen on the control plate surface (67±8%) (FIG. 6F, Table 11). The average percentage of cells expressing osteocalcin (n =3) in these experiments are shown in Table 11. The percentage of cells in Table 11 are reported as mean standard deviation.

TABLE 11

| Cell Culture Conditions | Cells Expressing Osteocalcin (%) |
|---|---|
| Plate | 67 ± 25 |
| PAGES | 26 ± 22 |
| PAGES + 50% BaSO₄ | 25 ± 19 |
| PAGES + 20% αTCP | 32 ± 26 |
| PAGES + 50% SrCO₃ | 53 ± 26 |

PAGES Provides an Appropriate Fill when Injected into a Vertebral Body

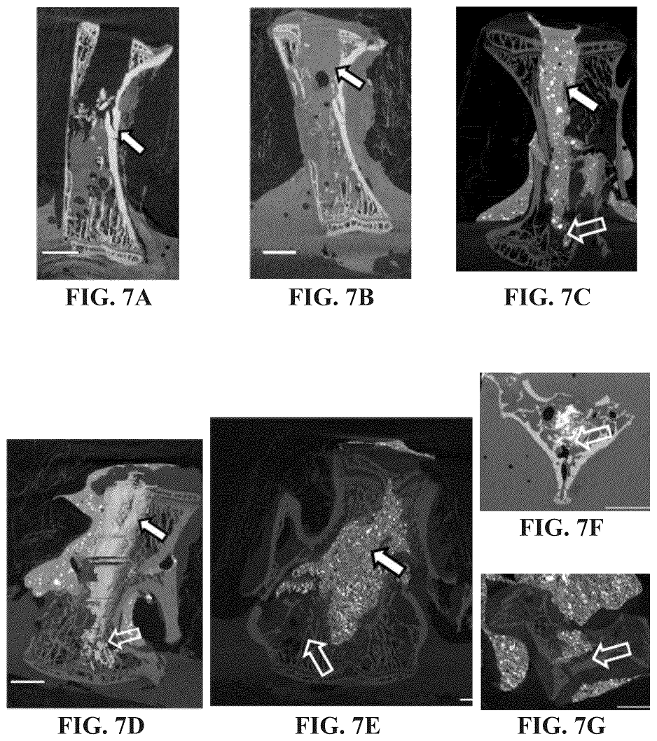

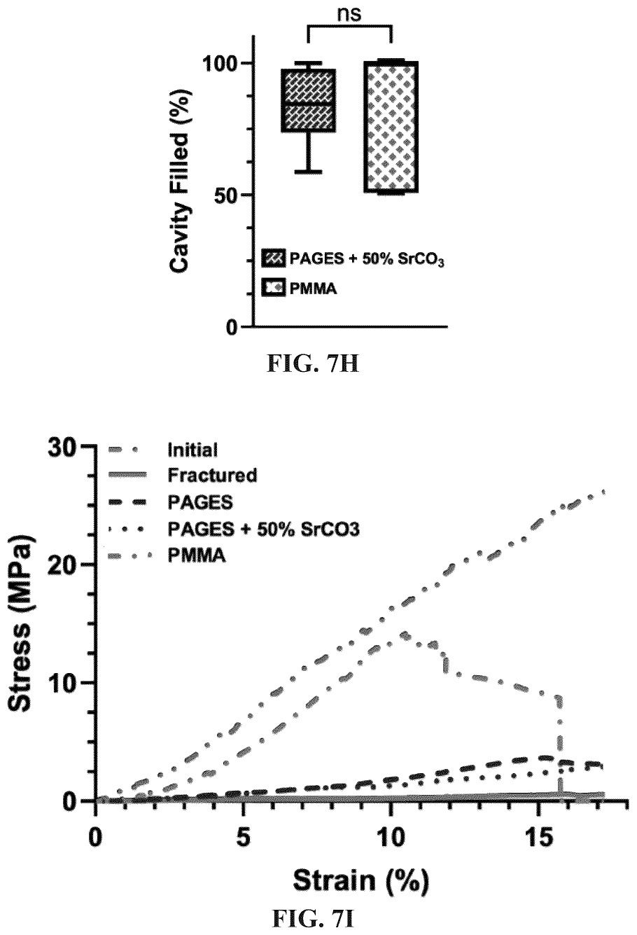

FIGS. 7A-J show the results of using DP₂₉ PAGES polymer at 60% crosslinking as a vertebral augmentation material in an ex vivo rabbit vertebral augmentation procedure. FIGS. 7A-G are microcomputed tomography (CT) images of rabbit vertebra during ex vivo vertebral augmentation (all scale bars represent 3 mm). The solid arrows highlight the location of a drilled cavity and the hollow arrows represent porous bone tissue that can be filled with augmentation material. FIG. 7A is a CT image of broken rabbit vertebra and FIG. 7B is a CT image of broken rabbit vertebra filled with PAGES with no additive. FIGS. 7C and F are CT images of rabbit vertebra filled with PAGES with 50% strontium carbonate (SrCO₃). FIG. 7D is CT image of rabbit vertebra filled with PAGES with 50% SrCO₃ with teal color displaying infill of PAGES into the porous bone structure. FIGS. 7E & 7G) are CT images of rabbit vertebra filled with poly(methyl methacrylate) (PMMA), the current clinical standard for vertebral augmentation. FIG. 7H is a graph showing the percentage of a drilled cavity in a rabbit vertebra that is filled with augmentation material using standard augmentation techniques (n=13 for PAGES+SrCO₃, n=6 for PMMA). FIG. 7I is a graph showing the averaged compressive stress-strain curves for rabbit vertebra initially, after being fractured, or after being fractured and filled with augmentation material (n=8, n=6 for PMMA). FIG. 7J is a graph showing the maximum compressive stress at break for rabbit vertebra initially, after being fractured, or after being fractured and filled with augmentation materials (n=8, n=6 for PMMA). The plotted data in FIGS. H, I, J shows the mean with error bars representing standard error of the mean. Box plots in FIGS. 7 I, J show median, first quartile, third quartile, maximum, and minimum. The degree of statistical significance determined using unpaired t-test or Tukey's multiple comparisons test with *p<0.05, **p<0.01, ***p<0.001, ****p<0.0001.

Pre-fractured rabbit vertebra (FIG. 7A) were filled with DP₂₉ PAGES augmentation material with and without SrCO₃ following a clinically relevant drilling procedure with a self-tapping kyphoplasty drill. Qualitatively, after filling the fracture, vertebrae were more stable and loose fragments were adhered to the bone. There was appropriate filling of the drilled vertebra with interdigitation of the polymer into the porous structure of the bone (FIGS. 7C-G, open arrows). Volumetric analysis through microcomputed tomography (CT) demonstrated that PAGES with SrCO₃ augmentation material fills drilled spaces equally as well as PMMA does, with an average of 84% of the drilled space filled when using either material (FIG. 7H). This analysis could not be performed for PAGES without ceramic because its radiographic density was too similar to the surrounding bone in the CT scans, demonstrating that the addition of SrCO₃ did increase radiopacity (FIG. 7B-C). Mechanical testing performed on the vertebra both before and after filling demonstrated an increase in the mechanical strength of the filled vertebra in comparison to the fractured vertebra, although the filled strength was below that of the native vertebra for all PAGES samples (FIGS. 7I-J). The ultimate strength of PMMA filled vertebra was higher than the native vertebra but did not reach statistical significance (FIGS. 7I-J, Tables 12 and 13).

The average percentage of drilled cavity in a rabbit vertebra filled with augmentation material using standard augmentation techniques (n=13 for PAGES+50% SrCO₃, n=6 for PMMA) are shown in Table 12. The percentage of cavity filled in Table 12 are reported as mean standard deviation.

TABLE 12

| Augmentation Material | Cavity Filled (%) |
|---|---|
| PAGES + 50% SrCO₃ | 84 ± 14 |
| PMMA | 84 ± 26 |

The average maximum compressive strength of tested rabbit vertebra are shown in Table 13. All measurements for initial (n=21) and fractured (n=22) vertebra mechanical properties were combined. (PAGES and PAGES+SrCO₃ composite (n=8). PMMA (n=6)). The compressive strength values in Table 13 are displayed as mean±95% Confidence Interval.

TABLE 13

| Vertebra Treatment | Maximum Compressive Strength (kPa) |
|---|---|
| Initial | 12915 ± 2545 |
| Fractured | 677 ± 418 |
| PAGES | 4255 ± 2803 |
| PAGES + 50% SrCO₃ | 4288 ± 1885 |
| PMMA | 29582 ± 14356 |

Crosslinked PAGES networks with SrCO₃ meet many of the requirements for a percutaneously applied bone augmentation material. The addition of SrCO₃ makes the polymer radiopaque with a radiographic density higher than the surrounding bone, making it easily visible with μCT imaging (FIG. 7A-G). Crosslinked PAGES samples demonstrated a peak compressive strength in the range of a native human vertebral body (1-6 MPa) with increased strength following the addition of either BaSO₄ or SrCO₃ (FIG. 4). There was some restoration of ex vivo rabbit vertebral body strength following filling with PAGES both with and without added SrCO₃ compared to the strength of the vertebra immediately following fracture (FIGS. 7A-J). The addition of SrCO₃ also increased osteoblast differentiation of MSCs cultured on PAGES samples, demonstrating that the SrCO₃ maintains its expected biological effects when incorporated into the crosslinked PAGES networks (FIG. 6A-F). The average procedural time for vertebroplasty is 31 minutes and for kyphoplasty is 40 minutes. (See, e.g., M. Dohm et al., “A randomized trial comparing balloon kyphoplasty and vertebroplasty for vertebral compression fractures due to osteoporosis,” AJNR. American Journal of Neuroradiology 35 (2014) 2227-2236, https://doi.org/10.3174/AJNR. A4127, the disclosure of which is incorporated herein by reference in its entirety). The time to gelation of 60% crosslinked DP₂₉ PAGES samples is approximately 17 minutes at physiological temperature and occurs over an appropriate duration for a typical procedure (FIG. 6). PAGES is a degradable polymer but maintains strength during degradation and has a long degradation time, which will help continuously support the bone throughout the healing process (FIG. 5). This is particularly important in the vertebral augmentation population as these patients tend to be older and take longer to heal bony injuries. (See, e.g., D. Clark, M. Nakamura, T. Miclau, R. Marcucio, “Effects of Aging on Fracture Healing,” Current Osteoporosis Reports 15 (2017) 601. https://doi.org/10.1007/S11914-017-0413-9, the disclosure of which is incorporated herein by reference in its entirety). Overall, crosslinked PAGES composites show promise as a bone augmentation material for percutaneous vertebral augmentation.

This work shows that crosslinked PAGES samples have tunable mechanical properties (FIG. 5). One area of future study will focus on increasing the stiffness of the PAGES composites to more closely mimic bone. Currently, PAGES samples have a higher strain at break than and a lower maximum compressive stress than native bone (FIG. 7E), which could cause additional stress on the surrounding bones following implantation. Future efforts will explore changing the crosslinker to increase stiffness of the network.

The addition of SrCO₃ is well known to encourage bone deposition in vivo and osteoblastic differentiation in vitro. Future work will explore the effects of PAGES implantation in vivo and aims to determine if the addition of SrCO₃ improves bone deposition. Sr²⁺ releasing bone scaffolds have been previously studied in mice and have been found to promote osteogenesis, reduce osteoclast functionality, and help resolve inflammation. (See, e.g., A. H. Lourenço, A. L. Torres, D. P. Vasconcelos, C. Ribeiro-Machado, J. N. Barbosa, M. A. Barbosa, C. C. Barrias, C. C. Ribeiro, “Osteogenic, anti-osteoclastogenic and immunomodulatory properties of a strontium-releasing hybrid scaffold for bone repair,” Materials Science and Engineering: C 99 (2019) 1289-1303. https://doi.org/10.1016/J.MSEC.2019.02.053, the disclosure of which is incorporated herein by reference in its entirety). Another area of additional investigation will explore the release of Sr²⁺ from the PAGES with SrCO₃ samples to determine how much Sr²⁺ is released over time.

Current vertebral augmentation is limited by the lack of degradability and cell interaction with PMMA augmentation material as well as the mechanical property mismatch between PMMA and native bone. The reported solvent free, injectable PAGES polymer composite system, particularly the SrCO₃ composites, showcases an encouraging novel bone augmentation material with many advantages including appropriate bone filling, promotion of osteoblastic differentiation, crosslinking in a time frame consistent with current clinical practice, and maintenance of mechanical strength while degrading.

### EXAMPLES

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

Tetrahydrofuran and toluene (HPLC Grade) were purchased from Sigma Aldrich and were dried using a Pure Solv MD 5 INERT solvent purification system. Propargyl alcohol was purchased from Sigma Aldrich and distilled over calcium hydride prior to use. Chloroform, hexanes, n-Butyl-sec-butyl magnesium 0.7 M solution in hexanes, 2,6-di-tert-butyl-4-methylphenol (BHT), allyl glycidyl ether, trimethylolpropane tris (3-mercaptopropionate), methyl methacrylate, polymethyl methacrylate, and alpha tricalcium phosphate (αTCP) were purchased from Sigma Aldrich and were used as received. Succinic anhydride, strontium carbonate (SrCO₃), barium sulfate (BaSO₄), and benzoyl peroxide were purchased from Thermo Scientific and were used as received. N,N dimethyl-p-toludine was purchased from TCI Chemicals and used as received.

### Example 1

Synthesis of Mg(BHT)₂(THF)₂ Catalyst

Mg(BHT)₂(THF)₂ was synthesized as described previously. (See, e.g., Wilson, J. A.; Hopkins, S. A.; Wright, P. M.; Dove, A. P. “‘Immortal’ Ring-Opening Polymerization of co-Pentadecalactone by Mg(BHT)₂(THF)₂.” Polym. Chem. 2014, 5 (8), 2691-2694. https://doi.org/10.1039/C4PY00034J, the disclosure of which is incorporated herein by reference in its entirety). Prior to synthesis, all glassware was oven dried at 150° C. and put under nitrogen atmosphere conditions using standard Schlenk line techniques. Briefly, n-butyl-sec-butyl magnesium 0.7 M solution in hexanes was combined with 2,6-di-tert-butyl-4-methylphenol (BHT) and tetrahydrofuran (THF) at room temperature under nitrogen atmosphere conditions. After reacting for 2 hours, excess THF was removed under reduced pressure and the catalyst was dried for an additional 24 hours under vacuum. Due to our familiarity of the polymerization and the one-pot synthetic approach for the catalyst and polymer, the catalyst is not isolated for most reactions, so a nearly quantitative yield is assumed in future calculations.

### Example 2

### General Procedure for the Synthesis of Poly(Allyl Glycidyl Ether Succinate).

Poly(Allyl Glycidyl Ether Succinate) (PAGES) was synthesized using a ring opening copolymerization (ROCOP) of succinic anhydride and allyl glycidyl ether with propargyl alcohol as the monofunctional initiator in toluene with a Mg(BHT)₂(THF)₂ catalyst as described previously. (See, e.g., Segal, M. I.; Bahnick, A. J.; Judge, N. G.; Becker, M. L. Synthesis and Solvent Free D LP 3D Printing of Degradable Poly(Allyl Glycidyl Ether Succinate). Angewandte Chemie International Edition 2025, 64 (2), e202414016. https://doi.org/10.1002/anie.202414016, the disclosure of which is incorporated herein by reference in its entirety). The polymerizations were conducted in the same glassware used to synthesize the Mg(BHT)₂(THF)₂ catalyst; thus this is a one-pot catalyst and polymer reaction.

PAGES polymers with different degrees of polymerization (DPₙ) were synthesized by adjusting the stoichiometry of monomers to initiator. The amount of toluene added to the reaction was adjusted to result in a total reaction concentration of 8 M. Catalyst loading was determined using a 0.02 molar ratio of catalyst to succinic anhydride. Prior to synthesis, all glassware was oven dried at 150° C. and put under nitrogen gas using standard Schlenk line techniques. The reaction was maintained at 80° C. while being stirred under nitrogen gas. The monomer conversion was monitored in crude samples from the reaction mixture using ¹H-NMR in CDCl₃. Following the polymerization, PAGES was purified via precipitation into hexanes and filtration.

### Example 3

### Poly(Allyl Glycidyl Ether Succinate) (PAGES) DP₂₉

Mg(BHT)₂(THF)₂ (15.00 g, 0.025 mol, 0.6 eq) was dissolved in dry toluene (327 mL) in a 1 L round bottom flask at 40° C. While stirring, propargyl alcohol (2.4 mL, 0.041 mol, 1.0 eq), allyl glycidyl ether (154.5 mL, 1.354 mol, 33 eq), and succinic anhydride (123.19 g, 1.231 mol, 30 eq) were added to the reaction flask. The temperature was increased to 80° C. The monomer conversion was first monitored when the reaction was no longer cloudy, indicating that the succinic anhydride had fully solubilized.

Post-purification ¹H NMR spectra revealed the following resonance shifts (ppm): ¹H NMR (600 MHz, CDCl₃): δ 7.27, 5.86, 5.28, 5.20, 4.69, 4.34, 4.22, 3.99, 3.56, 2.65, 2.49. SEC: MW=10.1 kg/mol, Mn=8.6 kg/mol. TGA: T_{d}=318° C. DSC: T_{g}=−34° C. (See, FIGS. 8, 10, 11, 13, 14; Table 1)

### Example 4

### Poly(Allyl Glycidyl Ether Succinate) (PAGES) DP₄₈:

Mg(BHT)₂(THF)₂ (15.00 g, 0.025 mol, 0.94 eq) was dissolved in dry toluene (327 mL) in a 1 L round bottom flask at 40° C. While stirring, propargyl alcohol (1.5 mL, 0.026 mol, 1.0 eq), allyl glycidyl ether (154.5 mL, 1.354 mol, 51.7 eq), and succinic anhydride (123.19 g, 1.231 mol, 47 eq) were added to the reaction flask. The temperature was increased to 80° C. The monomer conversion was first monitored when the reaction was no longer cloudy, indicating that the succinic anhydride had fully solubilized.

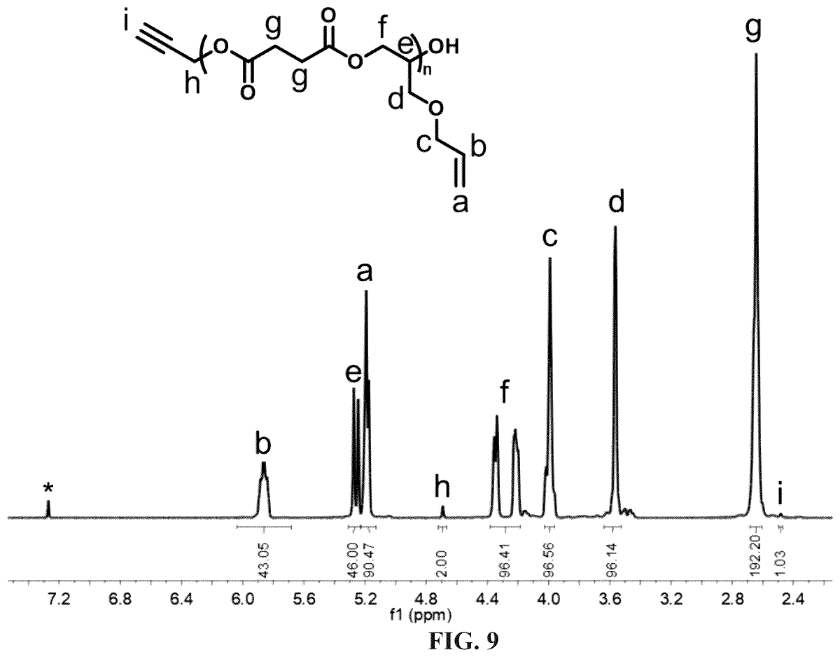

Post-purification ¹H NMR spectra revealed the following resonance shifts (ppm): ¹H NMR (600 MHz, CDCl₃): δ 7.27, 5.86, 5.27, 5.19, 4.69, 4.34, 4.22, 3.99, 3.56, 2.64, 2.48. SEC: M_{w}=12.7 kg/mol, Mₙ=10.8 kg/mol. TGA: T_{d}=317° C. DSC: T_{g}=−30° C. (See, FIGS. 9, 10, 12, 13, 14; Table 1)

### Example 5

### Nuclear Magnetic Resonance (NMR) Spectroscopy.

¹H NMR (600 MHz) were recorded using a Bruker Avance III spectrometer. Chemical shifts were recorded in parts per million (ppm) relative to the TMS reference resonance at δ=0.0 ppm. Proton ¹H NMR was used to monitor reaction conversion and determine the degree of polymerization. Degree of polymerization was determined by comparing the δ=4.7 ppm resonance of the initiator to the δ=4.3 ppm resonance of the polymer. (See FIGS. 8, 9; Table 1) Example 6

### Size-Exclusion Chromatography (SEC).

Number average molecular mass (Mₙ), weight average molecular mass (M_{w}), and molar mass distribution (D) were determined using a TOSOH Bioscience HLC-8320 GPC with a TSKgel GMHHR-N mixed bed column an RI and MALs detectors in tetrahydrofuran (THF) with eluent flowing at 0.35 mL/min and injection volume of 25 μL. M_{w} was calculated based on MALS detector traces using concentration, Ð was calculated using RI detectors, and subsequently Mₙ was derived from these values. (See, Table 1).

### Example 7

### Differential Scanning Calorimetry (DSC).

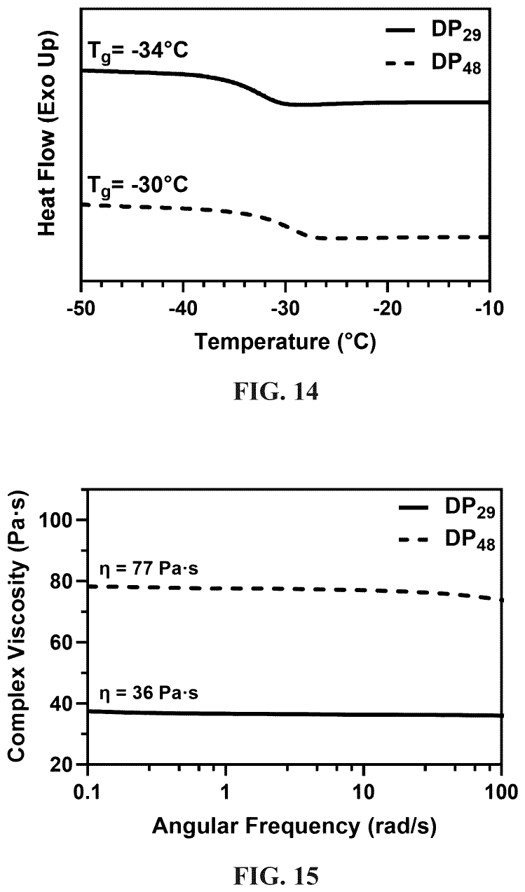

Thermal analysis was performed on a TA Instruments Discovery DSC 250. Each sample was prepared by loading 10 mg of material into an aluminum DSC crucible. The temperature range scanned was −60° C. to 70° C. at a 10° C./min ramp rate under N₂ gas flow. Two cycles were completed: the first to reset the material's thermal history and the second to collect representative data. All measurements are reported against a sealed, empty reference crucible. Glass transition temperature (T_{g}) was determined as the midpoint of the transition in the second heating cycle. (See, FIG. 14; Table 1).

### Example 8

### Thermogravimetric Analysis (TGA).

Thermal analysis was performed on a TA Instruments Discovery TGA 550. Each sample was prepared by loading 10 mg of material onto a platinum TGA pan. The temperature was ramped to 100° C. at 10° C./min, held isothermally for 30 minutes, equilibrated at 30° C., and finally ramped from 30° C. to 600° C. at 10° C./min all under N₂ gas flow. Degradation temperature (T_{d}) was determined as the temperature at 10% mass loss. (See, FIG. 13, Table 1).

### Example 9

### Attenuated Total Reflection Fourier Transform Infrared (ATR-FTIR) Spectroscopy.

ATR-FTIR spectra were collected using a Thermo Scientific Nicolet iS50 FT-IR spectrometer with a built-in iS50 diamond ATR. Liquid samples were placed directly onto the ATR crystal using a pipette. Solid samples were placed on the ATR crystal and then secured using a lever to ensure proper contact between the solid samples and the crystal. The number of Fourier transforms was set to 32 for both background and sample spectra collection. The results of the ATR-FTIR analysis are shown in FIG. 1B.

### Example 10

### Scanning Electron Microscopy (SEM).

Samples were mounted onto stubs using carbon tape and sputter coated using a Denton Desk V Sputter-Coater with gold at a coating rate of 1.39 nm/min for 60 seconds. SEM was performed on a Thermo Fisher Scientific Apreo S. Magnification varied between 500× and 100× magnification. The resulting images were analyzed using NIH FIJI software. (See, FIG. 21).

### Example 11

### Energy-Dispersive X-Ray Spectroscopy (EDS).

EDS was conducted using Oxford Instruments X-Max-N 150 SDD, samples were analyzed at 10 kV accelerating voltage and 0.8 nA beam current. Elemental maps were combined and overlayed with SEM images using NIH FIJI software color merge feature. (See, FIG. 21).

### Example 12

### Rheology: Viscosity.

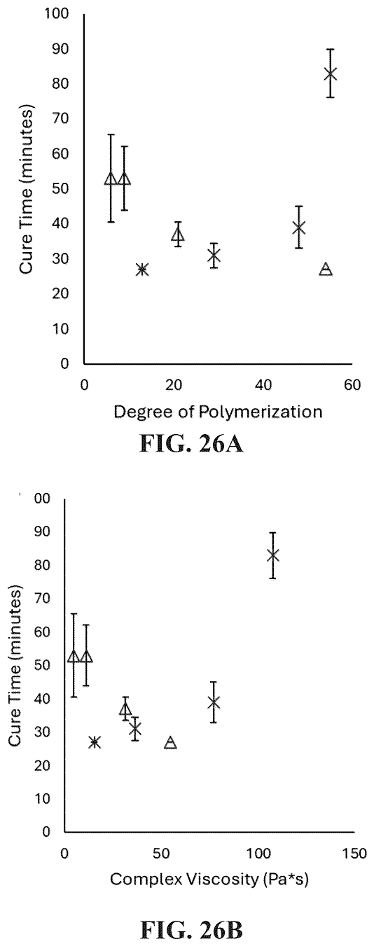

The complex viscosity of PAGES polymers were measured (n=1) using a TA Instruments Discovery HR-3 Hybrid Rheometer. Samples were loaded on 20 mm parallel plate geometry with a 0.2 mm gap. A trim gap of 50 m was used to ensure proper distribution of each sample between plates. The complex viscosity was collected over a frequency sweep from 0.1 rad/s to 500 rad/s at 10% strain and 25° C. All measurements took place within the linear viscoelastic regime for these materials as determined using a prior stain sweep. The viscosity of samples was reported as the complex viscosity at 10 rad/s. (See, FIG. 26B; Table 1).

### Example 13

### Rheology: Gel Point Determinations.

Polymer gel points were determined for each composition (n=3) and crosslinking percentage at both 25° C. and 37° C. using a TA Instruments Discovery HR-3 Hybrid Rheometer. Samples were prepared by mixing polymer and crosslinker according to the specified theoretical molar crosslink percentage, which is defined as the percentage of allyl groups in the polymer that react with thiol groups present in the crosslinker, in a glass vial under ambient conditions using a spatula until homogenous. Samples were then loaded on 20 mm parallel plate geometry with a 0.2 mm gap. A trim gap of 50 m was used to ensure proper distribution of each sample between plates. Samples were conditioned to the measurement temperature for 120 s prior to the experiment beginning. The storage modulus, loss modulus, and tan(6) were recorded over frequency sweeps repeated every 6 minutes from 0.1 rad/s to 500 rad/s at 1% strain. All measurements took place within the linear viscoelastic regime for these materials as determined using a prior stain sweep. Data are reported as the storage and loss moduli as a function of time with 1 measurement of each moduli reported from each frequency sweep at 10 rad/s and time being the total elapsed time to halfway through each frequency sweep. The gel point was calculated in two ways: 1) the time of the intersection of the storage and loss modulus and 2) the time at which tan(6) became independent of frequency, as determined by the most constant frequency sweep which was calculated as the frequency sweep with the lowest ratio of tan(6) standard deviation to average tan(6). (See, Table 3).

### Example 14

### Compression Testing.

Compression measurements were completed (n=5) for DP₂₉ and DP₄₈ polymers at each crosslinking percentage and composite formulation (n=6) using an Instron Model 5965 material testing system (Norwood, MA) equipped with parallel metal compression plates and a 1 kN load cell. Samples were prepared by mixing polymer and crosslinker according to the specified theoretical molar crosslink percentage, which is defined as the percentage of allyl groups in the polymer that would react with thiol groups in crosslinker, in a glass vial under ambient conditions using a spatula until homogenous. The respective ceramic fillers were added to the neat polymer prior to addition of crosslinker according to the specified wt. % of the polymer mass. Samples were then placed in freeform silicone molds to cure overnight under ambient conditions. Samples were removed from molds and cut into 5 mm×5 mm×10 mm rectangular prisms using a razor blade. Samples were left under ambient conditions to continue to cure until being tested at specified timepoints of 1, 7, 14, and 28 days. Sample dimensions were confirmed by measurement using calipers prior to compression testing. Samples were subjected to uniaxial compressive forces at a strain rate of 1.2%/min until failure. Data is reported as compressive stress-strain curves. Maximum compressive stress is reported as the maximum stress recorded in the test. Strain at break is reported as the strain at the maximum stress recorded in the test. Compressive modulus was determined as the slope between 5-7% strain on the stress-strain plots. (See, e.g., M. M. Panjabi, A. A. I. White, Basic Biomechanics of the Spine, Neurosurgery 7 (1980) 76; and T. Brown, R. J. Hansen, A. J. Yorra, “Some Mechanical Tests on the Lumbosacral Spine with Particular Reference to the Intervertebral Discs: A Preliminary Report,” JBJS 39 (1957) 1135, the disclosures of which are incorporated herein by reference in their entirety). Shear modulus was determined as the slope of the Neo-Hookean fit (stress versus (λ−λ⁻²)) of the stress-strain plots. (See, FIGS. 3A-C, 4A-F, 5A-F, 7I-J, 18, 19, 22-24; Tables 3-8, 13).

### Example 15

### Degradation of PAGES Polymers and Composites.

In Vitro degradation measurements of PAGES and PAGES composites (10 mm×5 mm×5 mm rectangular prisms) were performed in physiological conditions (PBS, pH 7.4) or under accelerated conditions (1 M HCl). The sample blocks were cast in a silicone mold and cut to size with a razor blade 4 hours after crosslinker addition. Sample mass and compressive strength were repeatedly measured over the study. Time points for measurement of mass changes were 6 hours, 1 day, 7 days, and then weekly, while compression time points were acquired at 6 hours, 1 day, 1 week, 2 weeks, 3 weeks, 4 weeks, 6 weeks, 8 weeks, 10 weeks, 12 weeks, 14 weeks, and 18 weeks. Prior to mechanical testing, samples were measured dimensionally with calipers and those numbers were used for stress and strain calculations. Testing was performed from 5-10% strain with a strain rate of 1%/min using a Discovery DMA 850 (TA Instruments). Data was collected both when loading and unloading. Compressive modulus was determined as the slope between 5-7% strain on the stress-strain plots. (See, e.g., M. M. Panjabi, A. A. I. White, Basic Biomechanics of the Spine, Neurosurgery 7 (1980) 76; and T. Brown, R. J. Hansen, A. J. Yorra, “Some Mechanical Tests on the Lumbosacral Spine with Particular Reference to the Intervertebral Discs: A Preliminary Report,” JBJS 39 (1957) 1135, the disclosures of which are incorporated herein by reference in their entirety). (FIGS. 5A-F, 13; Tables 9 and 10).

### Example 16

### Rat Mesenchymal Stem Cell Culture.

Primary Rat Mesenchymal Stem Cells (MSCs) were maintained in adherent culture in T75 flasks (Thermo Fisher Scientific) and fed every 2-3 days with MSC media (minimum essential media alpha (MEMα) (Cytiva) plus 10% v/v Fetal Bovine Serum (Gibco) and 1× Antibiotic/Antimycotic (Gibco)). Cells were passaged using 0.25% trypsin when they reached 70-90% confluency and reseeded at 5,000 cells/cm². (See, FIGS. 6A-F).

### Example 17

### Osteoblast Differentiation.

MSCs were seeded at 5,000 cells/cm² in 48-well plates (ThermoFisher Scientific). The wells were coated with a thin layer of PAGES with crosslinker prior to gelation. Plates were sterilized under UV light for 1 hour and left in the culture hood overnight prior to seeding. Plates were washed twice with MSC media prior to cell seeding. Cells were fed every 2-3 days while in culture. (See, FIG. 6F; Table 11).

### Example 18

### Cell Staining and Imaging.

After 17 days in culture, plates were washed twice with sterile PBS. Cells were fixed with 4% paraformaldehyde (Boster) shaking at room temperature for 20 minutes. Cells were then permeabilized with 1% Triton-X 100 (Promega) for 15 min on a shaker at room temperatures. Cells were incubated with 3% BSA blocking buffer (ThermoFisher Scientific) for 1 hour on the shaker at room temperature or overnight at 4° C. Cells were incubated with mouse anti-hOsteocalcin (R&D biosystems) at 5 ug/ul in 1% BSA blocker buffer overnight at 4° C. Plates were washed 3 times with 1% BSA. Cells were then incubated in donkey anti-mouse antibody conjugated to NL557 (R&D Biosystems) diluted 1:200 in 1% BSA for 1 hour at room temperature in the dark. Plates were then washed 2× with 1% BSA and then incubated for 15 minutes with DAPI (AAT Bioquest). Plates were then imaged using a Keyence BZ-X710 fluorescent microscope (Itasca, IL). Images were then analyzed using Cell Profiler to determine the percentage of nuclei that co-localized with osteocalcin staining. Three images were acquired per well and they were averaged together for a well average. Three wells were used for each condition and three independent seedings were measured. (See, FIGS. A-F).

### Example 19

### Rabbit Vertebral Testing.

Rabbit vertebrae were acquired from animals who were used for other experimental procedures that had already been sacrificed. Vertebra were separated from one another and the posterior elements were removed with Rongeurs leaving only the vertebra body. One end of the vertebral body was then embedded in 2-4 cm of PC-clear epoxy resin. The vertebral was then drilled with a self-tapping bone drill with a 3 mm outer diameter (Merit Medical) along its longitudinal axis. Embedded bones were then imaged with micro-computed tomography as detailed below. Following imaging, bones were soaked for at least 24 hours in PBS at 4° C. prior to mechanical testing. (See, FIGS. 7A-G).

### Example 20

### Vertebra Compression Studies.

Rabbit vertebra both before and after filling with polymer were crushed on an Instron Model 5965 material testing system. Wet vertebra were preloaded with 5 N and were then tested at a 1.2%/min compression rate. Stress was calculated as Force/(bone volume/height). Bones were compressed to failure. Following initial fracture, the bone fragments (if any) were placed back into anatomic alignment and the fracture bone was compressed again to determine post-fracture strength. Post-fracture testing was performed without preload and was stopped when the resin block started to impact the force curve or 30% strain was reached. Filled vertebra compression was also performed without preload and was stopped at failure. Compression rate was unchanged for all tested samples. (See, FIGS. 7H, I, J).

### Example 21

Micro-Computed Tomography (μCT).

CT scans were completed using a Nikon XTH 225 ST instrument. Voxel size was 15-18 μm3. X-ray projections were taken in a 360 degree scan. The x-ray energy was 155 kilovolts (kV). Beam power was determined by the voxel size where beam wattage was equal to the voxel value. Scans were reconstructed in Nikon software and volumetric analysis was performed in Avizo software. Avizo was used to determine the bone volume of the vertebral body and height which was used to calculate the average bone area for the sample. This average bone area was used to calculate the stress in mechanical testing. Volumetric analysis was also used to calculate the volume of the cavity created in the bone and how much PAGES+SrCO₃ was filled into the cavity. (See FIGS. 7A-G).

### Example 22

### PMMA Resin Preparation.

PMMA crystals were milled using a cryomill (Retsch) with 2 5-minute cycles at 30/s yielding a fine white powder. Solid components were combined using the following ratio: 88% PMMA, 10% BaSO₄, and 2% benzoyl peroxide. Liquid components were combined using the following ratio: 98.5% methyl methacrylate and 1.5% n,n-dimethyl-p-toludine. Solid and liquid compounds were then combined in a 1:1 w/v ratio to yield PMMA. Clinical PMMA from Merit Medical was prepared using the instructions on the package insert. The resulting PMMA was used for Example 23.

### Example 23

Ex Vivo Kyphoplasty: Filling Rabbit Vertebra with PAGES or PMMA Augmentation

Materials. Once the vertebral bodies were fractured and the mechanical properties had been obtained, they were filled with PAGES both with and without SrCO₃ or with PMMA. PMMA used for rabbit vertebra was from a clinical kyphoplasty kit (Merit Medical). To fill the vertebra bodies, PAGES and crosslinker were mixed in a glass vial and then transferred to a syringe 15 minutes after the addition of crosslinker. The syringe was then connected to an 18 gauge needle which was placed into the drilled cavity in the vertebra body at multiple angles to attempt to get filling into the medullary space of the bone. PAGES augmentation material was added to the bone until it was visible at the top of the drilled hole and the polymer could no longer be injected through the needle. For vertebra that had multiple fragments, the fragments were placed in anatomic position prior to polymer filling. The filled vertebrae were then stored at 37° C. in PBS for 7 days prior to mechanical testing. The vertebra were imaged with μCT 1-2 days after being filled with polymer. (See, FIGS. 7E, G, H, I, J; Table 5).

### Example 24

### Statistical Analysis.

Statistical analysis was performed using Prism GraphPad software and Excel. Sample averages were compared using an ordinary one-way ANOVA, two-way ANOVA (for cellular data), or repeated measures ANOVA (for degradation and rabbit vertebra data). Statistical significance being determined as a calculated p-value <0.05. Threshold values for levels of statistical significance are as follows: *p<0.05, **p<0.01, ***p<0.001, ****p<0.0001. Sample averages were compared to each other using a Tukey's multiple comparisons test with statistical significance being determined as a calculated p-value <0.05. Threshold values for levels of statistical significance are as follows: *p<0.05, **p<0.01, ***p<0.001, ****p<0.0001.

### Example 25

Synthesis of Mg(BHT)₂(THF)₂ Catalyst

Glassware was dried overnight at 150 C and placed under nitrogen atmosphere with standard Schlenk line techniques. Mg(BHT)₂(THF)₂ was synthesized by reacting n-butyl-sec-butyl magnesium 0.7 M solution in hexanes with 2,6 di-tert-butyl-4-methyphenol (BHT) and tetrahydrofuran (THF) at room temperature under nitrogen atmosphere for 4 hours. Excess THF was removed using vacuum and the catalyst was dried for an additional 48 hours under vacuum.

### Example 26

### Synthesis of Poly(Allyl Glycidyl Ether Succinate) (PAGES)

PAGES was synthesized using the ring opening copolymerization (ROCOP) of succinic anhydride and allyl glycidyl ether using either 1,6-hexanediol (2-arm) or propargyl alcohol (1-arm) as the initiator in toluene at 15 M total concentration with the Mg(BHT)₂(THF)₂ catalyst. Polymerization was carried out in the same glassware that was used for catalyst synthesis, making this a one-pot reaction. The reaction was performed at 80 C under a nitrogen atmosphere until the reaction transitioned from cloudy to clear indicating the polymerization of all succinic anhydride monomer. Allyl glycidyl ether was used in excess to ensure polymer end group control. Different degrees of polymerization were obtained by varying the ratio of initiator to monomers succinic anhydride and ally glycidyl ether.

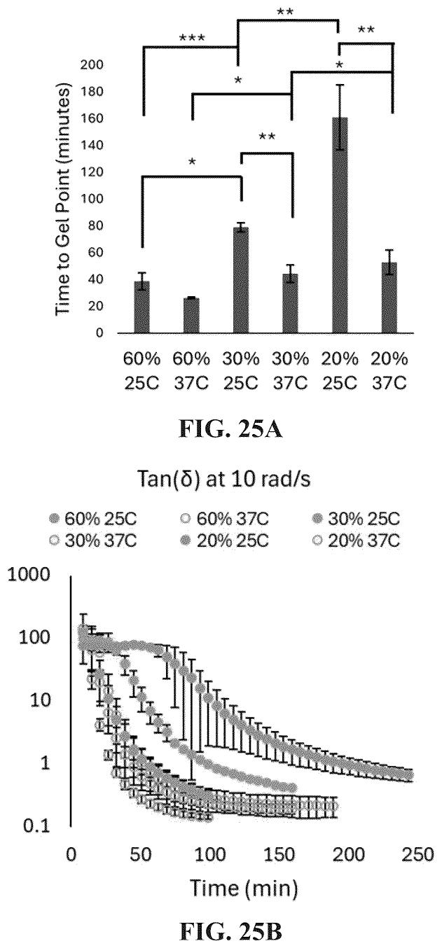

Reaction progression was monitored with crude ¹H NMR in CDCl₃. PAGES was purified using precipitation into hexanes and silica plug filtration. (See, FIGS. 25A-B)

### Example 27

### Nuclear Magnetic Resonance (NMR) Spectroscopy

¹H-NMR (600 MHz) were recorded using a Bruker Avance III spectrometer. Chemical shifts were recorded in parts per million (ppm) relative to the TMS reference resonance at δ=0.00. Integrations of the δ=4.3 ppm resonance of the polymer in comparison to the resonances of the initiators allowed for determination of degree of polymerization (DPₙ).

### Example 28

### Rheology

Gelation time was measuring using a TA instruments Discovery HR-3 Hybrid Rheometer. Samples were prepared by combining neat PAGES with trimethylolpropane tris(3-mercaptopropionate) crosslinker. If a composite additive (BaSO₄, SrCO₃, α-tricalcium phosphate (αTCP)) was used, it was added prior to crosslinker addition. Crosslink percentage was determined based on the molar ratio of terminal alkenes in the PAGES polymer (1 per monomer) and the thiols in the crosslinker (3 per crosslinker) and is expressed as the theoretical percentage of alkenes reacted. The PAGES-crosslinker mixture was loaded onto 20 mm parallel plates 5 minutes after crosslinker addition for 25 C samples or 3 minutes after crosslinker addition for 37° C. samples. 37 C sample loading timing was to allow for temperature equilibration prior to frequency sweeps starting. Frequency sweeps were then performed every 6 minutes from 0.1-500 rad/s with a 0.2 mm gap. Gel point was defined as the sweep where the tan(6) standard deviation/average was minimized and time to gel point expressed as the time of the middle frequency (10 rad/s) of the identified sweep. (See, FIGS. 25B, 26A-B).

### Example 29

### Bulk Polymer Rectangular Prisms

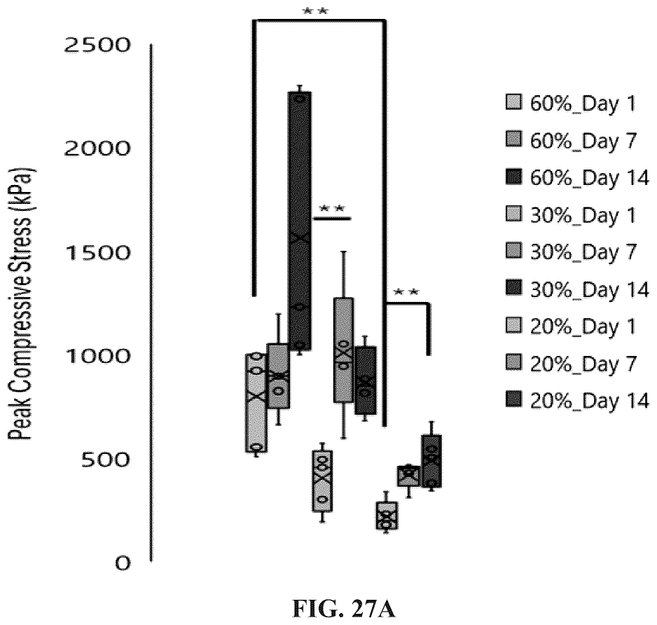

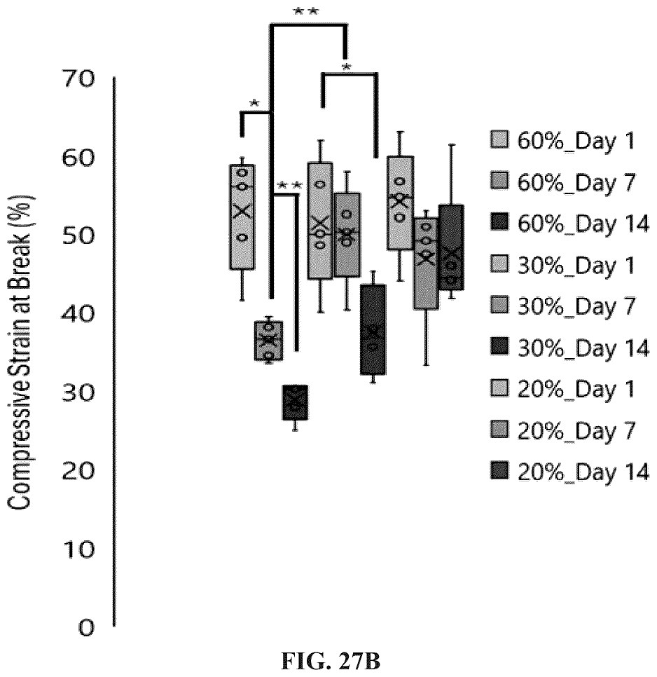

Rectangular prisms were produced to test the bulk polymer mechanical properties. PAGES was first mixed with any additives and then crosslinker was added at different ratios. Prisms were cast in silicone molds for 24 hours. The larger prisms were cut down to 5 mm×5 mm×10 mm using a razor blade prior to testing. Prism mechanical properties were tested on Day 1, Day 7 and Day 14. Until testing, the prisms were stored on the benchtop at room temperature. (See, FIGS. 27A-B, 28A-B).

### Example 30

### Compression Testing

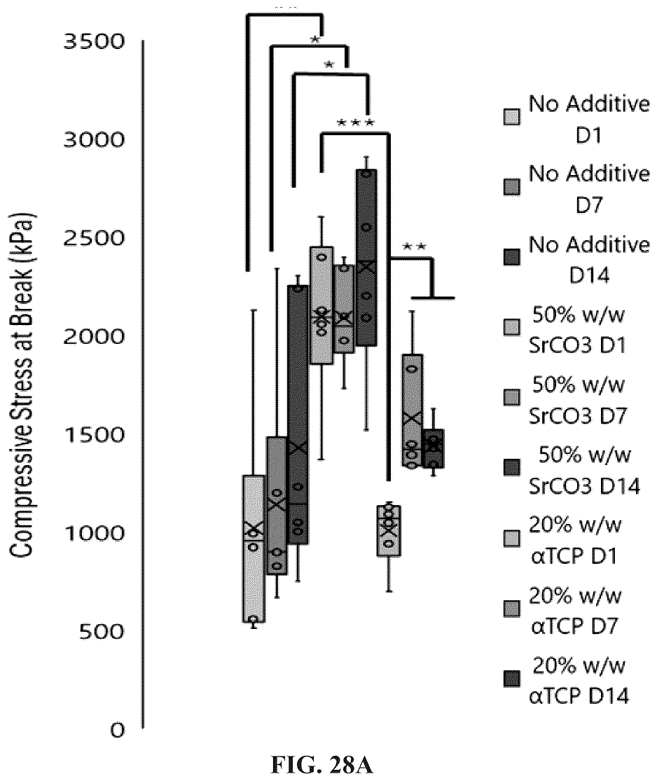

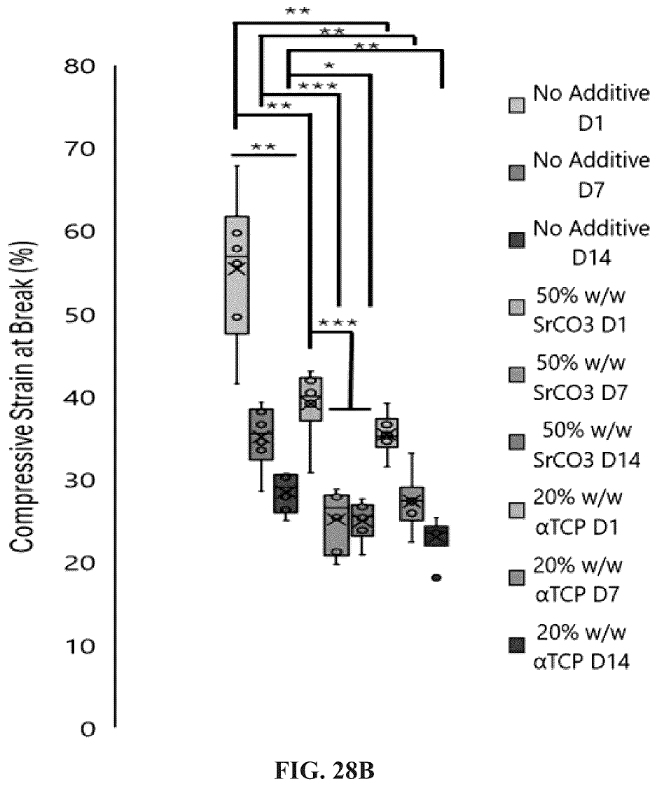

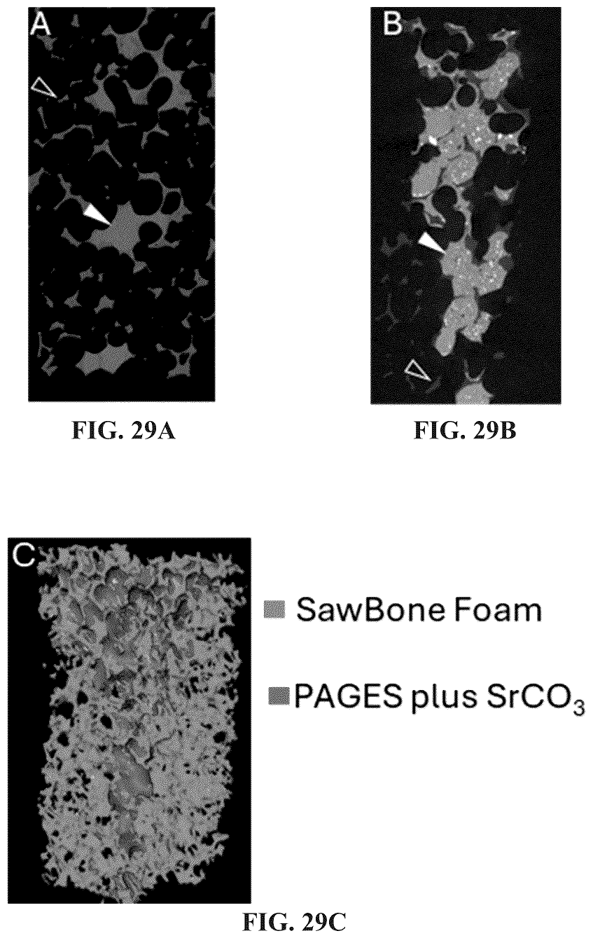

Compression testing was performed on an Instron Model 5965 material testing system (Norwood, MA) using parallel metal compression plates and a 1 kN load cell. Approximately 5 mm×5 mm×10 mm samples were compressed at a rate of 1.2%/min until failure. All samples were measured with calipers prior to compression and those measurements were used for stress and strain calculations and to determine compression rate. (See, FIGS. 28A-B).

In light of the foregoing, it should be appreciated that the present invention significantly advances the art by providing a solvent free, injectable, radiopaque composite network that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A solvent free, injectable, radiopaque composite network comprising:
a poly(allyl glycidyl ether succinate) copolymer comprising one or more allyl functional groups and a residue of a mono, di, or multi-functional initiating alcohol;
a multi-functional thiol crosslinking compound; and
a plurality of radiopaque ceramics or metal salt molecules.

2. The solvent free, injectable, radiopaque composite network of claim 1, wherein said poly(allyl glycidyl ether succinate) copolymer is the reaction product of succinic anhydride, allyl glycidyl ether, and a mono or di-functional initiating alcohol, and is formed by ring-opening polymerization using Mg(BHT)₂(THF)₂ as a catalyst.

3. The solvent free, injectable, radiopaque composite network of claim 1, wherein the poly(allyl glycidyl ether succinate) copolymer has a single poly(allyl glycidyl ether succinate) copolymer chain covalently bonded to the residue of a mono-functional initiating alcohol.

4. The solvent free, injectable, radiopaque composite network of claim 1, wherein said poly(allyl glycidyl ether succinate) copolymer has two poly(allyl glycidyl ether succinate) copolymer chain covalently bonded to the residue of a di-functional initiating alcohol.

5. The solvent free, injectable, radiopaque composite network of claim 3, having the formula: 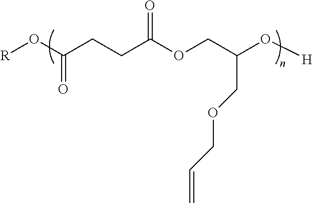
where n is an integer from about 10 to about 60 and R is a residue of an initiating alcohol.

6. The solvent free, injectable, radiopaque composite network of claim 4, having the formula: 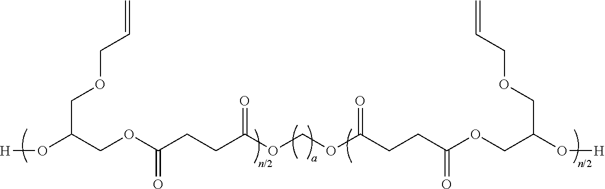
where n is an integer from about 10 to about 60 and where a is an integer from about 2 to about 12.

7. The solvent free, injectable, radiopaque composite network of claim 1, wherein said multi-functional thiol crosslinking compound selected from the group consisting of trimethylolpropane tris(3-mercaptopropionate), trimethylolpropane tris (3-mercaptopropionate), bis(3-mercaptopropyl) succinate, pentaerythritol tetrakis(3-mercaptopropionate), isosorbide bis(3-mercaptopropionate), 2,2′-((diisopropylsilanediyl)bis(oxy))bis(ethane-1-thiol), and combinations thereof.

8. The solvent free, injectable, radiopaque composite network of claim 1, wherein said multi-functional thiol crosslinking compound has the formula:

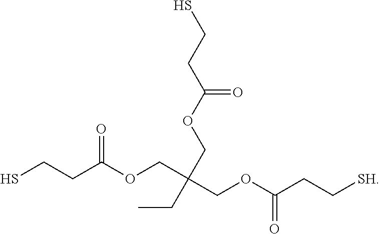 [SMILES: CCC(COC(=O)CCS)(COC(=O)CCS)COC(=O)CCS]

9. The solvent free, injectable, radiopaque composite network of claim 1, having a consumption percentage of from about 35% to about 60 of said solvent free, injectable, radiopaque composite network.

10. The solvent free, injectable, radiopaque composite network of claim 1, wherein said plurality of radiopaque ceramics or metal salt molecules comprise a radiopaque ceramic or metal salt of strontium, barium, calcium, or a combination thereof.

11. The solvent free, injectable, radiopaque composite network of claim 1, wherein said plurality of radiopaque ceramic molecules comprise strontium carbonate.

12. The solvent free, injectable, radiopaque composite network of claim 1, wherein said plurality of radiopaque ceramic molecules comprise from about 30% to about 80 by mass of said solvent free, injectable, radiopaque composite network.

13. The solvent free, injectable, radiopaque composite network of claim 1, wherein said solvent free, injectable, radiopaque composite network is from about 30% to about 60%-crosslinked after curing.

14. The solvent free, injectable, radiopaque composite network of claim 1, having a maximum compressive stress of from about 2 MPa to about 5 MPa after crosslinking.

15. The solvent free, injectable, radiopaque composite network of claim 1, having a maximum strain at break of from about 25 MPa to about 50 MPa after crosslinking.

16. The solvent free, injectable, radiopaque composite network of claim 1, wherein said solvent free, injectable, radiopaque composite network is injectable prior to curing.

17. The solvent free, injectable, radiopaque composite network of claim 1, wherein said solvent free, injectable, radiopaque composite network has a compressive strength comparable to human vertebra.

18. The solvent free, injectable, radiopaque composite network of claim 1, wherein the radiopaque composite network is seeded with mesenchymal stem cells.

19. A method for making the solvent free, injectable, radiopaque composite network of claim 1, comprising:
A) preparing a poly(allyl glycidyl ether succinate) copolymer comprising one or more allyl functional groups and a residue of a mono, di, or multi-functional initiating alcohol;
B) adding a plurality of radiopaque ceramics or metal salt molecules poly(allyl glycidyl ether succinate) copolymer and mixing to ensure even distribution of said plurality of radiopaque ceramics or metal salt molecules throughout said poly(allyl glycidyl ether succinate) copolymer; and
C) adding a desired quantity of a multi-functional thiol crosslinker and allowing the mixture to crosslink to form the solvent free, injectable, radiopaque composite network of claim 1.

20. The method for making the solvent free, injectable, radiopaque composite of claim 28 further comprising:
D) seeding the radiopaque composite network (step C) with mesenchymal stem cells.
